(19) United States
(12) Patent Application Publication
(10) Pub. No.: US 2026/0232870 A1
(43) Pub. Date: Aug. 13, 2026

(54) 3D POLYCATENATED ARCHITECTED MATERIALS AND RELATED COMPOSITIONS METHODS AND SYSTEMS

(71) Applicants: CALIFORNIA INSTITUTE OF TECHNOLOGY, Pasadena, CA (US); LAWRENCE LIVERMORE NATIONAL SECURITY, LLC, Livermore, CA (US)

(72) Inventors: Wenjie Zhou, Pasadena, CA (US); Chiara Daraio, South Pasadena, CA (US); Sujeeka Nadarajah, Pasadena (CA); Xiaoxing Xia, Livermore, CA (US); Liuchi Li, Baltimore, MD (US); Hujie Yan, Pasadena, CA (US)

(21) Appl. No.: 19/390,223

(22) Filed: Nov. 14, 2025

Related U.S. Application Data
(60) Provisional application No. 63/720,646, filed on Nov. 14, 2024.

(51) Int. Cl. A61L 27/52; F03G 7/06; F16F 7/12

(52) U.S. Cl. CPC A61L 27/52; F03G 7/06147; F03G 7/0616; F16F 7/12

(57) ABSTRACT

Described are 3D polycatenated architected materials, systems, and methods. The material comprises discrete unit particles mechanically interlocked to form a 3D network patterned after a crystalline topology. This structure provides a tunable dual mechanical response, transitioning from a fluid-like regime to a solid-like regime at a programmable “critical jamming strain.” Methods are disclosed for designing the material using a closed-loop digital twin, manufacturing the material via additive processes, and preconditioning the material. Methods and systems for actuating the material using mechanical, electrostatic, magnetic, thermal, or environmental triggers are also disclosed. Further disclosed are kits-of-parts for assembling the material, and end-user systems, such as impact protection systems, soft robotics, and medical implants, incorporating the material.

### CROSS-REFERENCE TO RELATED APPLICATIONS

The present application claims priority to U.S. Provisional Application No. 63/720,646 filed on Nov. 14, 2024, the disclosure of which is incorporated herein by reference in its entirety. The present application may also be related to U.S. patent application Ser. No. 17/748,626 filed on May 19, 2022, titled “Structures with Tunable Stiffness”, the disclosure of which is incorporated herein by reference in its entirety.

### STATEMENT OF GOVERNMENT GRANT

This invention was made with government support under Grant No. W911NF-22-2-0109 awarded by the Army and under Grant No. DE-AC52-07NA27344 awarded by the National Nuclear Security Administration. The government has certain rights in the invention.

### FIELD

The present disclosure relates generally to architected materials, and more specifically to three-dimensional, polycatenated architected materials and related compositions, methods and systems.

### BACKGROUND

Architected materials derive their bulk properties from the geometric arrangement of their internal structural elements, rather than from their constituent material alone. This design approach has led to materials with remarkable behaviors [2] [11] [16]-[29], such as high strength-to-weight ratios or negative Poisson's ratios [12] [13].

Despite advancements in this field obtaining an architecture material structure that is both fully cohesive in 3D while also composed of discrete particles, and which can provide controllable kinematic degrees of freedom is still challenging.

### SUMMARY

Provided herein are 3D polycatenated architecture materials and related compositions, methods and systems, which enable obtaining architecture materials which are both fully cohesive, due to inter-layer linkages, and internally reconfigurable, due to the kinematic degrees of freedom of its discrete particles.

According to a first aspect a structure is described, comprising: a plurality of unit particles, each unit particle comprising a particle body formed from one or more structural members and defining one or more interlocking openings at one or more nodes.

In the structure of the present disclosure, the plurality of unit particles are interconnected to form a three-dimensional network patterned after a crystalline network topology and comprising inter-layer linkages.

In the structure of the present disclosure, the interconnection is a mechanical interlock formed by an interlocking structural member of one unit particle passing through an interlocking opening of an adjacent unit particle at a node, such that the particle bodies are topologically intertwined and cannot be separated without being cut or broken.

In the structure of the present disclosure, an interconnection among the plurality of unit particles provides three-dimensional cohesion to the structure, the three-dimensional cohesion arising from mechanical interlocks at nodes, including inter-layer linkages. The adjacent particle bodies are not bonded and are topologically inseparable absent cutting or fracture.

According to a second aspect a method and systems are described for engineering a structure according to the present disclosure. The method comprises: determining a particle body geometry, the particle body geometry being formed from one or more structural members and defining one or more interlocking openings at one or more nodes; determining a 3D crystalline network topology and determining one or more structural member dimensions a constituent material, and a structural scale.

The method for engineering a structure in accordance with the present disclosure further comprises selecting the particle body geometry and the 3D crystalline network topology the one or more structural member dimensions, the constituent material, and the structural scale to achieve a target conversion state activatable by a selected trigger; and forming a digital representation of a structure by creating a plurality of unit particles with the particle body geometry, the unit particles being mechanically interlocked with each other in the 3D crystalline network topology by an interlocking structural member of one unit particle passing through an interlocking opening of an adjacent unit particle.

The system for engineering a structure according to the present disclosure is described. The system comprises: a topology/geometry library, a parametric modeling engine, a contact-aware simulation module (e.g., DEM/LS-DEM or rigid multibody with non-smooth contact), and a UI for selecting variables and reporting predicted

$${γ}_{s}^{*} / {ϵ}_{c}^{*} .$$

According to third aspect a method and a system for actuating a structure of the present disclosure are described.

The method for actuating a structure of the present disclosure comprises: providing a structure of the present disclosure; applying an electrostatic charge to the plurality of unit particles; and causing the structure to convert from a first state to a second state due to electrostatic repulsion between the unit particles, wherein the conversion is reversible upon neutralizing the electrostatic charge.

The system for actuating a structure of the present disclosure is an electrostatic actuator system, comprising: one or more structures according to the present disclosure; and an electrical voltage source communicatively coupled to the conductive material of the plurality of unit particles, wherein the electrical voltage source is configured to apply an electrostatic charge to the unit particles, causing the structure to reversibly change shape to reversibly convert from a first state to a second state.

According to fourth aspect an architected material is described, comprising: a plurality of discrete particle bodies, each particle body formed from one or more structural members. In the architected material of the present disclosure the plurality of particle bodies are mechanically interlocked to form a three-dimensional network with inter-layer linkages.

In the architected material herein described, the architected material is configured to exhibit a dual mechanical response, transitioning from a first state to a second state, the transition occurring at a programmable critical jamming strain.

According to a fifth aspect, a method for manufacturing an architected material of the disclosure. The method comprises: forming a plurality of unit particles by an additive manufacturing process, each unit particle comprising a particle body formed from one or more structural members and defining one or more interlocking openings at one or more nodes; wherein the forming is performed such that an interlocking structural member of one unit particle passes through an interlocking opening of an adjacent unit particle at a node, thereby creating a mechanically interlocked three-dimensional network patterned after a crystalline network topology, and optionally, applying a conductive coating to the plurality of unit particles to configure the architected material for electrostatic actuation.

According to a sixth aspect a method and a system are described for actuating an architected material of the present disclosure.

The method for actuating an architected material of the present disclosure comprises: providing the architected material of the present disclosure, wherein the plurality of discrete particle bodies are coated with a conductive material; applying an electrostatic charge to the plurality of discrete particle bodies; and causing the architected material to reversibly change shape due to electrostatic repulsion between the particle bodies.

The system for actuating an architected material of the present disclosure is an electrostatic actuator system, comprising: the architected material of the present disclosure; and an electrical voltage source communicatively coupled to the conductive material of the plurality of unit particles, wherein the electrical voltage source is configured to apply an electrostatic charge to the unit particles, causing the structure to reversibly change shape.

According to a seventh aspect, a method and a system for actuating an architected material of the present disclosure are described.

The method for actuating an architected material of the present disclosure comprises: providing the architected material of the present disclosure; applying a mechanical force to the plurality of discrete particle bodies; and causing the architected material to convert from a first state to a second state by inducing a strain that exceeds a programmable critical jamming strain, wherein the conversion is reversible upon removal of the mechanical force.

The system for actuating an architected material of the present disclosure is a mechanical actuator system, comprising: the architected material of the present disclosure; and a mechanical actuator communicatively coupled to the architected material, wherein the mechanical actuator is configured to apply the mechanical force to the plurality of discrete particle bodies to cause the architected material to reversibly convert from the first state to the second state.

According to an eighth aspect, a method for actuating a structure is described, comprising: providing the structure, wherein the structure is formed from a constituent material responsive to a trigger selected from the group consisting of: a magnetic field, a thermal change, a solvent, humidity, and light; applying the trigger to the structure; and causing the structure to convert from a first state to a second state.

According to a ninth aspect, a system for actuating a structure is described, comprising: the structure, wherein the structure is formed from a constituent material responsive to the trigger; and an actuator configured to apply the trigger, the actuator selected from the group consisting of: a magnetic field generator, a heating element, a solvent source, a humidity source, and a light source.

According to a tenth aspect, a kit of parts for assembling a 3D polycatenated architected material is described. The kit comprises: a plurality of pre-fabricated, discrete unit particles, each unit particle comprising a particle body formed from one or more structural members and defining one or more interlocking openings at one or more nodes; wherein the plurality of unit particles are configured to be assembled such that an interlocking structural member of one unit particle passes through an interlocking opening of an adjacent unit particle, thereby forming a mechanically interlocked three-dimensional network. The kit may further comprise instructions for assembly.

According to an eleventh aspect, a method of preconditioning an architected material is described. The method comprises: providing the architected material; and applying a cyclic mechanical load to the architected material for one or more cycles, thereby causing the architected material to stabilize from an initial mechanical response to a steady-state mechanical response having a reduced hysteresis loop. This method may also be used to train a multi-stable structure to convert between stable configurations.

According to a twelfth aspect, end-user systems incorporating the structure or architected material are described. Such systems include, but are not limited to, an impact protection system (such as a helmet, body armor, or seismic damper), a soft robotic system, a morphing architecture, and a flexible medical implant.

According to a thirteenth aspect, methods of manufacturing the end-user systems are described. The methods comprise integrating or incorporating the structure or architected material as a functional component into the end-user system.

The 3D polycatenated architecture materials and related compositions, methods and systems, herein described, enable in several embodiments bridging the gap between continuous solid lattices and discrete granular materials. Conventional architected materials, which rely on rigidly connected trusses [1] [2] or plates [3] [4], have a fixed mechanical response and fail by fracture. Conversely, granular materials, while reconfigurable, are not cohesive and lack tensile strength without external confinement. By utilizing a 3D crystalline network of mechanically interlocked unit particles, 3D polycatenated architecture materials and related compositions, methods and systems, herein described, achieve a structure that is both fully cohesive, due to inter-layer linkages, and internally reconfigurable, due to the kinematic degrees of freedom of its discrete particles.

The 3D polycatenated architecture materials and related compositions, methods and systems, herein described in several embodiments enable programmable, dual mechanical behavior that transitions from a fluid-like state to a solid-like state. This transition is governed by a measurable threshold known as the Critical Jamming Strain. At strains below this threshold, the unit particles are free to rearrange, and the material exhibits a fluid-like regime. This regime is quantifiable by a near-zero shear modulus, observed as a stress plateau in quasi-static tests, and by a shear-thinning response in rheological tests, where the complex viscosity (η*) may decrease by several orders of magnitude (e.g., from approximately 10\hat{ }5 Pa·s down to 10\hat{ }2 Pa·s). At strains above the critical jamming strain, the particles' kinematic degrees of freedom are exhausted, causing them to jam. This marks the transition to a solid-like regime, which is quantifiable by a nonlinear, elastic stress-strain response, significant strain-stiffening, and shear-thickening behavior, where viscosity (η*) begins to increase from its minimum.

The 3D polycatenated architecture materials and related compositions, methods and systems, herein described, enable in several embodiments the material's function to be tuned for specific applications by using the critical jamming strain as a programmable parameter. This tunability is operated by the selection of the catenation topology. For example, a J-4-ring topology is structured for high shear flexibility, resulting in a high critical shear jamming strain

$$( {γ}_{s}^{*} )$$

in a range of approximately 30% to 70%, making it ideal for morphing applications. In contrast, a T-6-ring topology utilizes a “scissor” mechanism, resulting in a high critical compressive jamming strain

$$( {ϵ}_{c}^{*} )$$

in a range or approximately 10% to 28%, making it highly effective for compressive energy absorption. This solves the problem of fixed-property materials by providing a framework to program an anisotropic mechanical response.

The 3D polycatenated architecture materials and related compositions, methods and systems, herein described allow in several embodiments to further refine the tunability of the material by operating the structural member dimensions, which provides fine-control over the critical jamming strain. This is quantifiable by the ratio of the structural member thickness (d) to the interlocking opening diameter (D), or (d/D). The critical jamming strain is inversely related to this d/D ratio. A low d/D ratio (e.g., approximately 0.14) creates larger clearances, which increases the kinematic DOFs and raises the critical jamming strain, resulting in a more flexible, fluid-like material. Conversely, a high d/D ratio (e.g., approximately 0.275) reduces clearances, which decreases the DOFs and lowers the critical jamming strain, resulting in a stiffer material that jams sooner.

The 3D polycatenated architecture materials and related compositions, methods and systems, herein described enable in several embodiments is scale-independent mechanical behavior. Because the material's properties are derived from its topology rather than its chemical composition, the dual-behavior and nonlinear stress-strain response are preserved across vastly different length scales. The qualitative mechanical behavior is shown to be consistent at the macro-scale (e.g., sample side lengths of approximately 24 mm) and at the micro-scale (e.g., sample side lengths of approximately 400 μm), representing a 60-fold reduction in scale.

The scale-independence mechanical behavior of the 3D polycatenated architecture materials and related compositions, methods and systems, herein described enables in several embodiments a mode of actuation in which at the micro-scale, the particle weight is dramatically reduced (e.g., by a factor of ~216,000) and the surface-area-to-volume ratio is high (e.g., ~60 times larger). This allows inter-particle forces, such as electrostatic repulsion, to dominate gravity. When a micro-scale structure is coated with a conductive material (e.g., approximately 300 nm of copper) and an electrostatic charge is applied, the mutual repulsion between particles causes the structure to rapidly expand from a compact to a deployed state. This actuation is fully reversible and rapid, with state transitions occurring in less than 0.1 seconds, providing a solution for stimuli-responsive materials and micro-scale soft robotics.

The 3D polycatenated architected materials and related compositions, methods and systems, herein described, can be used in connection with any applications wherein a material with programmable mechanical properties, high energy absorption, tunable stiffness, or stimuli-responsive behavior is desired. Exemplary applications comprise systems for energy absorption and impact protection, such as protective cases and shock absorbers for sensitive equipment, vehicle crash structures, aircraft impact protection, blast protection, seismic dampers, and personal protective equipment including helmets and body armor. Additional applications include stimuli-responsive and morphing architectures, such as components for soft robotics, military and defense systems, wearable technology, and flexible medical implants or grafts. At the micro-scale, the materials can be used for remotely actuated micro-devices or smart material systems, for example in aerospace applications for vibration attenuation in optical systems. Further applications include broad-band acoustic insulation. Additional uses and applications are also identifiable by a skilled person.

The details of one or more embodiments of the disclosure are set forth in the accompanying drawings and the description below. Other features, objects, and advantages will be apparent from the description and drawings, and from the claims.

### BRIEF DESCRIPTION OF DRAWINGS

FIGS. 1A-1B show an example configuration of the structure. FIG. 1A shows an example unit particle of the structure. FIG. 1B shows an example of interlocked unit particles.

FIG. 2 shows an example of the design strategy for PAMs. (FIG. 2 panels A-E) A typical design workflow of PAMs from a designated network: (Panel A) Network of the dia topology. (Panel B) Essential nodes and their connections with adjacent nodes in the dia network. Each pair of nodes are mapped with two tetrahedral particles catenating their vertices, while aligning their 3-fold axes. (Panel C) An extended D-4-TET PAM composed of tetrahedral particles. (Panel D) An array of exemplary PAM variants in addition to the catenated tetrahedra, namely Catalan tetrahedra (D-4-CT), icosahedra (D-4-ICO), cubes (D-4-cube), octahedrally arranged six-ring clusters (J-4-ring), tetrahedrally arranged four-ring clusters (T-6-ring), and tetrahedrally arranged four-hexagon clusters (T-6-HEX). The green line highlights the 3-fold symmetrical axis. (Panel E) A series of extended PAMs corresponding to the configurations from (D). (FIG. 2 Panels F-M) Generation of PAMs from designated particle geometry: (Panel F) A cuboctahedron (CO), catenated through its 4-fold (blue), 3-fold (green), and 2-fold (red) axes. (Panels J-M) Expanded PAMs from the catenations illustrated in panels G-I.

FIG. 3A-3B shows an example of gravity-induced relaxation and uniaxial compression of PAMs. (FIG. 3A Panels A-C) Relaxation of a series of J-4-ring PAMs with spherical domain boundaries, placed on a flat surface. When oriented along the <100> (Panel A), <110> (Panel B), and <111> (Panel C) crystallographic axes, they show different relaxed outline shapes. Scale bar: 2 cm. (FIG. 3A Panels D-G) Illustration and corresponding photos of expanded J-4-OCT, S-6/2-OCT, J-4-ring, and T-6-ring PAM samples. Scale bars: 1 cm. (FIG. 3B Panel H) Stress-strain plots of J-4-OCT and S-6/2-OCT PAMs under uniaxial compression. (FIG. 3B Panel I) Summary of normalized stiffness against measured volume fractions at 5%~10% strain for each PAM. (FIG. 3B Panel J) A comparison of the response of a T-6-ring sample under uniaxial compression between experiment and LS-DEM (Level Set-Discrete Element Method) simulation result. The inset shows the calculation of E* by fitting a polynomial to the simulated stress-strain response during the loading phase. (FIG. 3B Panel K) A visualization of the contact forces (blue being compressive ones and red being tensile ones) at three selected loading stages (see Panel J). Each contact force is represented by a cylinder, whose orientation is the force vector direction and whose length and radius are scaled according to the force magnitude. (FIG. 3B Panel L) Relation between E* and <Z>-Z₀ for four simulations considering different pre-loading configurations and loading strain ranges. All data can be reasonably represented by a power-law scaling (black dashed line) by setting Z₀=5.

FIG. 4 shows an example shear and rheology test of PAMs. (Panels A, B) PAM samples in shearing and rheological test, showing the deformation in response to corresponding loads. (Panels C, F) Stress-shear strain results for J-4-ring (Panel C) and T-6-ring (Panel F) PAMs, showing the transition from fluid-like to solid-like regime with the increase of shear strain. (Panels D, E, G, H) Rheology results of J-4-ring (Panel D) and T-6-ring (Panel G) PAMs under oscillatory amplitude sweep (Panel E) and frequency sweep (Panel H), with plots of storage modulus (G′), loss modulus (G″), and complex viscosity (η*) as a function of torsional strain and angular frequency. Dotted lines indicate the region where the tests were significantly affected by the instrument inertia effects. The blue shaded area in (Panels C, D, F, G) indicates the region where the PAMs exhibit fluid-like behavior, transitioning to solid-like behaviors as indicated by the red shaded area. (Panels I, K) Simulation results of the variation of η* as a function of angular frequency for J-4-ring (Panel I) and T-6-ring (Panel K) cylindrical samples. Insets show the mean contact number, <Z>, (left axis) and normalized granular temperature,

$$\frac{δ v}{{ω}_{c r i t} R}$$

(right axis) as a function of angular frequency. Here, ω_{crit} is the angular frequency at which the inflection of η* occurs in our simulations and R is the radii of the sample, used only for making δv dimensionless. (Panels J, L) Simulation results of the variation of

$$\frac{{η}^{*}}{δ v}$$

as a function of Z_{c}-Z for the J-4-ring (Panel I) and T-6-ring (Panel K) cylindrical samples. The black dashed lines are power-law fits using simulation data before the inflection of η* Insets show a magnified visualization of the main plot including a portion of the data after the inflection of η*.

FIG. 5 shows an example of programmable critical jamming strains of PAMs. (Panels A, C) The schematics illustrate the local ring arrangements under shearing and compressive loads for J-4-ring and T-6-ring PAMs, respectively. (Panel B) A summary plot displaying critical shear jamming strain

$$( {γ}_{s}^{*} )$$

against the critical compressive jamming strain

$$( {ε}_{c}^{*} )$$

for both types of PAMs containing rings with varied thicknesses. (Panels D-S) Images showing J-4-ring (Panels D-K) and T-6-ring (Panels L-S) PAMs at their corresponding critical jamming strains.

FIG. 6 shows an example of scale independence of PAMs and electrostatic actuation of μ-PAMs. (Panel A) Stress-strain curves of C-6-TT PAMs fabricated at different scales (60 times difference in all dimensions) and volume fractions (III>II>I). Inset shows a comparative summary of energy absorption capacities of PAMs at two scales. (Panel B, C) Snapshots of macroscale and microscale (Panel B)C-6-TT PAMs undergoing compression experiments at strains of 0%, 25%, and 50%. Scale bars: 1 cm (Panel B), 0.1 mm (Panel C). (Panels D-K) The J-4-ring μ-PAMs in various geometries including cubes and letters before and after electrostatic expansion. White regions in illustrations highlight the fixing area between the μ-PAMs and the substrate. These μ-PAMs, when subjected to electrostatic charges generated by a Van de Graaff generator, deploy from a relaxed natural state to an expanded state due to inter-particle electrostatic repulsion. Scale bars: 1 mm.

FIG. 7 shows an example Selected library of PAMs composed of single type of particles. Different tones highlight local structural features that may be visually challenging to distinguish, including particle geometries, orientations, and linking types. PAMs consist of more than one types of particles are not listed in this library.

FIG. 8A shows an example of tuning particle geometry of PAMs with the same topology. PAMs composed of octahedral (OCT) and cuboctahedral (CO) particles are linked in S-6/2 and J-4 topologies. By truncating octahedra into cuboctahedra, existing local contact mechanisms of PAMs can be drastically altered, namely transitioning from corner-to-corner locking into face-to-face locking or edge-to-edge locking.

FIG. 8B shows an example of replacing multiple particles with one (geometric transformations among T-6-ring, D-4-TET, and C-6-TT). On the left side, T-6-ring is divided into two sets of tetrahedral clusters (yellow and blue), arranged in a dia topology. Replacing each cluster with one tetrahedral particle results in a D-4-TET. On the right side, T-6-ring is divided into two sets of tetrahedral clusters (yellow and red), clusters arranged in a pcu topology. Replacing each cluster with one truncated tetrahedral particle results in a C-6-TT.

FIG. 9 shows an example relaxation behavior of PAMs under gravity. The global outlines of J-4-ring PAMs are designed as a cube composed of 540 ring particles (Panels A, B) and a sphere composed of 9414 ring particles (Panels C, D). Scale bars: 1 cm.

FIG. 10 shows an example Catenation configurations and fabrication of PAMs. (Panels A-D) Schematic representations of J-4-ring, J-4-square, T-6-ring, and T-6-HEX PAMs' local configurations, where J-4-ring and J-4-sqr have planar CN of 4, T-6-ring and T-6-HEX have planar CN of 6. (Panels E-H) Illustration and corresponding photos of expanded J-4-ring, J-4-SQR, T-6-ring, and T-6-HEX PAM samples. (Panels I-L) Schematic representations of J-4-OCT, S-6/2-OCT, D-4-TET, and C-6-TT PAMs' local configurations, where J-4-OCT has planar CN of 4, S-6/2-OCT has octahedral (Oh) CN of 6 and 2, D-4-TET has tetrahedral (Td) CN of 4, C-6-TT has Oh CN of 6. (Panels M-P) Illustration and corresponding photos of expanded J-4-OCT and S-6/2-OCT, D-4-TET, and C-6-TT PAM samples. Scale bars: 1 cm.

FIG. 11 shows an example of experimental setups for mechanical characterizations. (Panel A) Uniaxial compression test setup, (Panel B) Rheology test setup, (Panel C) Design and positioning of the mounting plate on the PAM specimen to secure the shear test sample in the Instron testing machine, (Panel D) Assembly of the mounting plate with the solid gripping plate integrated into the shear test specimen, and (Panel E) Shear test specimen positioned in the Instron machine utilizing the solid gripping plate and mounting plate. Scale bars: 1 cm.

FIG. 12 shows an example of uniaxial compression test of PAMs. (Panels A-F) Stress-strain results of six PAMs from cyclic loading (10%, 20%, 30%, 40%, and 50%) and direct loading (50%): (Panel A) T-6-ring, (Panel B) T-6-HEX, (Panel C)C-6-TT, (Panel D) J-4-ring, (Panel E) J-4-square, and (Panel F) D-4-TET. Circles indicate the onset of significant fatigue, calculated from the first derivative tests.

FIG. 13 (Panels A-H) shows an example of cyclic compression of all PAMs at same loading conditions (10% strain). A series of force-displacement response from cyclic compression tests on all discussed PAMs in this study at 10% strains illustrating the preconditioning of the PAMs during the initial loading cycles.

FIG. 14 shows an example of force-displacement response of the J-4-ring PAM under uniaxial compressive loading across various initial configurations. The force responses were measured using a triaxial force sensor, which recorded forces along the loading direction (Z-axis) and two perpendicular directions (X and Y axes). The panel of images displays the different initial configurations tested, including upright, random, sheared, and twisted arrangements. The plots clearly illustrate the influence of each configuration on the global force-displacement behavior along the Z-axis. Additionally, the forces recorded in-plane (X and Y directions) display a high degree of randomness.

FIG. 15 shows an example of three C-6-TT PAMs with different volume fractions. Three PAM structures are designed with the same particle size (truncated tetrahedra), only different in their beam thicknesses, with a ratio of 7:8:9. These PAM structures are fabricated at both macro- and microscale, in order to compare the scalability of PAMs' mechanical responses.

FIG. 16 shows an example of Results of E* using different polynomial fit to simulated stress-strain responses of T-6-ring sample. (Panels A, C, E, and G): Using polynomial of second, third, fourth and fifth degree, respectively, but all with Z₀=2 for the two “As is” cases. (Panels B, D, F, and H): Using polynomial of second, third, fourth and fifth degree, respectively, but all with Z₀=5 instead for the two “as is” cases. Z_{ref} is a constant selected as 1 kPa, used for the sole purpose of normalizing E* to be dimensionless.

FIG. 17 shows an example of additional particle-scale contact analysis from LS-DEM simulation of T-6-ring under uniaxial compression. (Panel A): Variation of mean “engaged” friction between particles as a function of cumulative strain. (Panel B): Variation of the Gini coefficient of contact forces as a function of cumulative strain.

FIG. 18 shows a representative rheology simulation output of J-4-ring under an excitation frequency of 360 rad/s. Left axis shows the variation of the shear stress (raw output in grey line while smoothed data in black solid line) as a function of time calculated at the top boundary. Right axis shows the imposed sinusoidal shear strain with an amplitude of 0.1.

FIG. 19 shows an example of simulated critical jamming strains of PAMS. (Panels A-P) Simulation results displaying J-4-ring (Panels A-H) and T-6-ring (Panels I-P) PAMs at their corresponding critical jamming strains.

FIG. 20 shows an example Simulated critical jamming strain of PAMs. The simulated critical jamming strain under both shear and compressive loading conditions is analyzed as a function of ring thickness and topology, as described in FIG. 5. The trends and values match the experimental results shown in FIG. 5, panel B.

FIG. 21 shows an example of maximal critical shear jamming strains of J-4-ring and T-6-ring PAMs. Top-down photographs show J-4-ring (left) and T-6-ring (right) PAMs, fixed with top and bottom gripping plates, horizontally placed on a flat surface. From top row to bottom row, PAMs are designed within the same domain, but with increasing numbers of smaller unit cells. Left columns show the natural state of the PAMs, while right columns show PAMs at their critical shear jamming strains.

FIG. 22 shows an example of a rheology test of simple cubic truss lattice. Experimental setup and results of an amplitude sweep test using a simple cubic truss lattice, with consistent configurations as the tests on PAMs. The test was automatically stopped at an oscillation strain of 5.6%, due to a catastrophic failure developed across one layer in the sample.

FIG. 23 shows an example of fabrication and electrostatic reconfiguration of μ-PAM. Top row shows the design, two-photon lithography, and plasma etching process of a J-4-ring PAM. Bottom panel illustrates Cu-plated μ-PAM samples attached to ITO-coated glass substrates, placed atop a Van de Graaff generator.

FIG. 24 shows an example graph of the effect of different friction coefficient in the LS-DEM simulation of PAM's rheological behavior.

FIG. 25 shows an example PAMs fabricated using varied material compositions. PAMs printed with three different materials and designs using powder laser sintering, a method ideal for support-free structures. The design and material variations result in different macroscopic properties, showcasing the versatility of PAMs.

FIG. 26 shows an example configuration of J-4-ring PAM Samples Under Structural Rotation. (Panel A) Illustrations showing the cubic boundary conditions of a J-4-ring PAM matrix being rotated at 0°, 15°, 30°, and 45°. (Panel B) Images of samples corresponding to each rotation angle depicted in (Panel A).

FIG. 27 (Panels A-D) shows an example of Force-Displacement Curves for Rotation Series of J-4-ring PAM Under Varied Shear Conditions. Shaded regions highlight the range of displacement where shearing force is near-zero.

FIG. 28 shows an example of Rheological Behavior of J-4-ring (Panel A) and T-6-ring (Panel B) PAMs Under Varying Strain Levels.

FIG. 29 shows an example Comparison of Complex Viscosity of J-4-rings at Dry State vs Fully Submerged in Water. (Panel A) An image showing the experimental setup for submerged rheology experiments. (Panel B) Comparison of complex viscosities of dry and submerged J-4-ring PAMs.

FIG. 30 shows an example Typical Axial Force Measurements in Rheology Experiments: (Panel A) Amplitude Sweep, (Panel B) Frequency Sweep.

FIG. 31 (Panels a and b) shows an example Variation of the Portion of Compressive Contact and Velocity Fluctuation as Functions of the Angular Frequency.

FIG. 32 shows an example schematic of a PAM design system.

FIGS. 33A and 33B show a prophetic example of magnetic field actuation, corresponding to Example 18. FIG. 33A illustrates the structure in its first, flexible state, absent a magnetic field.

FIG. 33B illustrates the structure converting to its second, stiff state upon the application of a magnetic field.

### DETAILED DESCRIPTION

Provided herein are 3D polycatenated architecture materials and related compositions, methods and systems, which enable obtaining architecture materials both fully cohesive, due to inter-layer linkages, and internally reconfigurable, due to the kinematic degrees of freedom of its discrete particles.

The term “architected material” as used herein indicates engineered solids whose bulk properties arise primarily from their internal geometry rather than their chemical composition. Accordingly, in architected materials, geometry, topology, and scale are engineered to control stiffness, strength, density, energy absorption, and other functional characteristics of the bulk material.

The term “topology”, as used herein, refers to the pattern of connectivity among structural elements within the material, independent of their exact size, shape, or orientation. In an architected material, topology defines which elements are connected to which others, and in what manner, such as the number of connections per node, their spatial arrangement, and the existence of interlocking or catenation relationships. Topology is therefore a connectivity map or network architecture that specifies how the components of the structure are interrelated.

Exemplary topologies suitable for use in architected materials comprise any periodic or aperiodic network that defines a pattern of connectivity among structural elements or unit cells. Topologies can be derived from crystalline lattice frameworks, graph networks, or tessellation schemes, and may exhibit cubic, tetrahedral, octahedral, or other symmetries.

Representative examples of three-dimensional crystalline or network topologies include: Diamond (dia) topology, comprising tetrahedrally coordinated nodes connected to four neighbors; Cubic (bcu, cP) or body-centered cubic (bcc) topologies, comprising nodes connected along orthogonal axes; Face-centered cubic (fcc) or octet topology, in which each node connects to twelve neighbors forming a close-packed lattice; Tetrahedral (T) and octahedral (O) frameworks, exhibiting threefold or fourfold symmetry axes; srs and pcu topologies derived from reticular chemistry, exhibiting chiral or non-chiral three-dimensional connectivity; Gyroid and Schwarz P- or D-surface topologies representing continuous triply periodic minimal-surface networks; J-, C-, or T-type topologies, denoting interlocking networks with four, six, or eight interconnections per particle, respectively, as in polycatenated architected materials, and additional topologies identifiable by a skilled person.

The term “geometry”, as used herein, refers to the specific shape, dimensions, and spatial configuration of the structural elements that constitute the architected material. Geometry defines the metric properties of each feature—such as lengths, radii, wall thicknesses, angles, and curvatures—and determines the form of the unit cell and its components (e.g., struts, plates, shells, or particle bodies).

Exemplary geometries suitable for forming the unit cells or particle bodies of architected materials comprise polyhedral, polygonal, toroidal, spherical, or shell-like forms. The geometry of each structural element defines its metric characteristics, including edge length, wall thickness, curvature, and overall symmetry.

Representative examples of geometries include: Tetrahedral, octahedral, cuboctahedral, and icosahedral polyhedra, suitable for forming node-centered or corner-sharing frameworks; Cubic or rectangular prismatic geometries, defining orthogonal strut alignments; Toroidal or ring-shaped geometries, providing apertures for interlocking or catenation; Spherical or ellipsoidal geometries, for isotropic packing and multi-directional rotation; Hexagonal, triangular, or Catalan polyhedra for dense or anisotropic tessellations; Plate or shell geometries, including curved or corrugated surfaces for enhanced stiffness-to-weight ratios; Hybrid geometries, combining toroidal and polyhedral elements to produce adjustable degrees of freedom and additional geometries identifiable by a skilled person.

The wording “Polycatenated Architected Materials”, as used herein, indicates a specific class of architected material. While a general architected material derives its properties from geometry, a polycatenated architected material is fundamentally distinguished by its discrete, non-bonded, and topologically interlocked structure. Instead of being a single monolithic or continuous body of rigidly connected trusses, plates, or shells, a polycatenated architected material is comprised of a plurality of discrete unit particles, or particle bodies. These particle bodies are interconnected to form a cohesive, three-dimensional network not by fused joints, chemical bonds, or adhesives, but by mechanical entanglement. This specific interconnection is achieved when an interlocking structural member of one unit particle passes through an interlocking opening of an adjacent unit particle at a node, forming a structure that is held together by its topology.

In polycatenated architected materials in the sense of the disclosure, the selected geometry determines the local degrees of freedom (DOF) and mechanical constraints of each unit cell. For example, toroidal or ring geometries allow relative rotation and sliding between adjacent particles, whereas polyhedral geometries provide higher stiffness and resistance to deformation. The geometry may be scaled or parameterized to adjust clearances, wall thickness, and surface curvature, thereby tuning the effective elastic, damping, or energy-absorption characteristics of the bulk material.

As used herein, a “unit particle,” “particle,” or “unit cell” refers to the primary, discrete, and repeating rigid body in the structure. Each unit particle comprises a “particle body” having a prescribed geometry, in embodiments herein described, the geometry of the particle body defines the three-dimensional form, symmetry, and spatial proportions of each discrete element constituting the architected material.

A particle body is of architected materials herein described are formed from one or more structural members and define one or more openings.

The term “structural member” as used herein refers to any portion of the particle body that provides material continuity and contributes to the mechanical integrity or shape of the particle. Structural members can include struts, ribs, struts, plates, walls, or curved shell segments that together form the outer contour or internal framework of the particle body. The size, cross-sectional profile, and orientation of the structural members determine the local stiffness, strength, and deformation characteristics of the particle body.

The term “opening” refers to any aperture, passage, slot, throat, or channel defined within or through the particle body that allows partial or complete insertion of a structural member or extension of another particle body. The opening can be circular, polygonal, or curvilinear in cross section and may extend fully through the particle body or terminate within it. Each opening provides an interlocking interface through which a corresponding structural member or strut of an adjacent particle passes to form a mechanical linkage. The spatial arrangement of the structural members and openings defines the overall geometry of the particle body and establishes the pattern of mechanical connectivity within the three-dimensional network of the architected material.

In embodiments herein described, the geometry of the unit cells and related structural members and openings defines specific regions on the particle body that serve as interconnection sites, referred to herein as “nodes” formed by interlocking structural members and interlocking opening.

As used herein, the term “node” refers to a defined region or portion of a particle body that functions as a mechanical interconnection site between adjacent particles within the architected material. Each node is characterized by the convergence or intersection of one or more interlocking structural members and one or more interlocking openings, thereby establishing a location through which mechanical engagement and load transfer occur. The node can be positioned at a corner, edge, face, or central portion of the particle body, depending on the selected geometry and topology of the unit cell.

The term “interlocking structural member” refers to a strut, arm, loop, or other projection that extends from the particle body and is configured to pass at least partially through an interlocking opening of an adjacent particle body. Each interlocking structural member can be integral with the particle body and can include straight, curved, or contoured segments sized to fit within the corresponding opening while maintaining a predetermined clearance gap that permits limited translational or rotational movement between interlocked particles.

The term “interlocking opening” refers to an aperture, slot, channel, or passage defined within or through the particle body at the node and dimensioned to receive an interlocking structural member of a neighboring particle. The geometry of the interlocking opening and the orientation of the interlocking structural member together define the mechanical constraint and degree of freedom present at the node. When a plurality of particle bodies are arranged in accordance with the predetermined topology of the architected material, the interlocking structural members of each particle extend through corresponding interlocking openings of adjacent particles, forming a three-dimensional network of mechanically catenated nodes that provides volumetric cohesion without the use of bonded or welded joints.

Accordingly, in architected materials herein described, one or more structural member of a unit cell can extend between, around, or across one or more openings and can terminate at or define a node, which serves as an interconnection site for mechanical engagement with an adjacent particle as will be understood by a skilled person upon reading of the present disclosure.

In some embodiments of the polycatenated architected material of the disclosure, the particle body can assume a polyhedral, toroidal, spherical, shell-type, or hybrid configuration, or any other geometry that provides the desired number and orientation of nodes and interlocking openings. Polyhedral geometries, such as tetrahedral, cubic, octahedral, cuboctahedral, and icosahedral forms, provide discrete faces and edges that can serve as load-bearing regions and establish directionally defined interlocking sites. Toroidal geometries, in which the particle body forms a continuous ring or annulus defining a central aperture, enable polycatenation by allowing the struts or extensions of adjacent particles to pass through and mechanically engage one another. Spherical or ellipsoidal geometries may include circumferential grooves, perforations, or necked regions configured to receive corresponding interlocking features of neighboring particles, allowing isotropic rotation or rolling contact within the network. Shell-type or cage-type geometries define open or partially enclosed frameworks of struts, ribs, or plates that reduce mass while maintaining structural stiffness.

In some embodiments, the particle body can exhibit a hybrid geometry combining characteristics of two or more of the foregoing, such as ring-polyhedron hybrids, polyhedral-spherical composites, or gyroidal cages having continuously curved surfaces. The selected geometry of the particle body determines the number and orientation of nodes and interlocking openings, the local distribution of mass and stiffness, and the mechanical degrees of freedom of each particle within the network. Toroidal and cage-type geometries generally facilitate rotational and translational motion between particles, whereas polyhedral geometries confer greater rigidity and energy absorption. By adjusting the geometric parameters of the particle body, including wall thickness, curvature, or opening size, the overall stiffness, density, and jamming response of the architected material may be tuned to achieve a desired functional performance.

In embodiments of architected material in the sense of the disclosure, the material typically comprises a periodic or aperiodic array of unit cells, each unit cell including a network of structural elements such as struts, plates, shells, or, as in embodiments herein, discrete particle-like elements.

In architected material of the disclosure the interconnection between particles is formed by a mechanical interlocking. In which two or more particle bodies are topologically intertwined such that they cannot be completely separated from each other without being cut or broken. This is achieved when an interlocking member, passes through an interlocking opening of an adjacent particle. at a node, of a particle body. This arrangement creates a cohesive, three-dimensional structure comprising inter-layer linkages that is held together by mechanical entanglement rather than by chemical bonds or fused joints as will be understood by a skilled person upon reading of the present disclosure.

In particular, in embodiments of polycatenated architected materials, topology includes the three-dimensional network of mechanical interlocks patterned after a crystalline lattice, and can incorporate inter-layer linkages between adjacent particles or unit cells. Changes in topology (e.g., changing a cubic linkage to a tetrahedral linkage, or converting bonded joints to interlocked joints) alter the fundamental mechanical response of the material even if the underlying geometry remains unchanged.

As used herein, a “patterned structure” refers to the physical, tangible embodiment of the architected material itself. The structure is described as “patterned” because its plurality of unit particles are not arranged randomly, but are instead organized and interconnected in a deliberate, spatially repeating design. This pattern, which arises from the specific placement and interconnection of each particle, is distinct from the random arrangement of particles in a conventional granular material or the uniform, continuous structure of a bulk monolithic material.

In embodiments of the invention, the “patterned structure” is “patterned after a crystalline lattice” (or “crystalline network topology”). This signifies that the abstract, periodic arrangement of the crystalline lattice is used as the design template to dictate the physical architecture of the material. In this translation from the abstract topology to the physical structure, the periodic locations of the “nodes” in the crystalline lattice correspond to the spatial locations of the “unit particles” (or “particle bodies”) in the patterned structure. Furthermore, the abstract connections or edges that join nodes in the crystalline lattice correspond to the “mechanical interlocks” (formed by “interlocking structural members” and “interlocking openings”) that physically and topologically link adjacent unit particles in the final patterned structure.

In some embodiments, the topology is patterned after a three-dimensional crystalline network and comprises inter-layer linkages between unit cells arranged in adjacent planes, thereby establishing volumetric mechanical connectivity. In other embodiments, the topology may be hierarchical, combining two or more lattice types or incorporating defect structures or disordered interconnections to tune mechanical response.

In architected material herein described, the connectivity, orientation, and dimensional proportions of structural elements of the material define the overall macroscopic behavior of the material as will be understood by a skilled person. Accordingly, two architected materials fabricated from identical base substances can exhibit substantially different mechanical or physical responses as a result of differing internal architectures as will be understood by a skilled person upon reading of the present disclosure.

In particular, the mechanical behavior of architected materials herein described are programmable design features, controlled by three primary structural variables: Catenation Topology, Particle Body Geometry, and Structural Member Dimensions.

The term “catenation topology” as used herein refers to the three-dimensional pattern of mechanical connectivity established among a plurality of particle bodies within the architected material. The catenation topology defines which particle bodies are interlocked with one another and the manner in which the interlocking occurs, including the number of connections per particle, the spatial orientation of those connections, and the symmetry of the resulting network.

In certain embodiments, the catenation topology is patterned after a three-dimensional crystalline lattice, such as a diamond, cubic, or tetrahedral network, in which the nodes of the lattice correspond to particle bodies and the lattice edges correspond to interlocking relationships. In some embodiments, the catenation topology can include inter-layer linkages extending between particles located in adjacent planes, thereby forming a volumetrically interlocked framework that maintains mechanical cohesion in all three spatial dimensions. Variations in the catenation topology, such as changes in the number of interlocks per particle or the angular orientation of the interlocking members, produce corresponding variations in the stiffness, energy absorption, and jamming response of the architected material.

Exemplary catenation topologies include lattice or network configurations designated as J, T, D, and C types, each describing a distinct pattern of interlocking between particle bodies. In a J-type topology, each particle body is interlocked with four neighboring particles arranged along generally orthogonal or three-fold symmetric axes, producing a framework suitable for high shear deformability and reversible jamming. A T-type topology may include six interconnections per particle arranged in a tetrahedral or hexagonal configuration, providing balanced compressive and shear strength. A D-type topology corresponds to a diamond-like lattice in which each particle engages four neighbors through corner-to-corner or edge-to-edge interlocks, producing an architecture characterized by high stiffness and strain-stiffening behavior. A C-type topology can include six or more interconnections arranged cubically or octahedrally, forming a dense and isotropic network. Other suitable catenation topologies may be derived from crystalline or graph-based networks, such as simple cubic, body-centered cubic, face-centered cubic, or gyroid frameworks, and can include hierarchical or hybrid forms combining two or more of the foregoing as will be understood by a skilled person. In embodiments herein described, the 3D crystalline network topology may be selected from various crystalline or network topologies, such as those described using Reticular Chemistry Structure Resource (RCSR) symbols [36]. Exemplary topologies include, but are not limited to, dia, pcu (including pcu-b), bcc/bcu, and fcc (octet). Further examples may include nbo-b, which can serve as a J-type analog, pbz/hxg, which can serve as a T-type analog, and srs. The topology may also comprise other related 3D periodic networks, or hierarchical and hybrid combinations thereof.

The term “particle body geometry” as used herein refers to the three-dimensional form, shape, and symmetry of each discrete element or unit cell component constituting the architected material. The particle body geometry defines the overall contour of the particle and the configuration of its structural members, walls, and openings. Exemplary geometries include polyhedral forms such as tetrahedral, cubic, octahedral, and icosahedral bodies; toroidal or ring-shaped bodies defining central apertures; spherical or ellipsoidal bodies optionally including grooves or recesses; cage-type or shell-type frameworks composed of interconnected struts or plates; and hybrid geometries combining multiple of the foregoing. The selected particle body geometry determines the number and orientation of nodes and interlocking openings available for engagement with neighboring particles and governs the degrees of freedom and deformation behavior of each particle within the catenated network.

Exemplary particle body geometries corresponding to the topologies of architected materials in the sense of the disclosure comprise polyhedral, toroidal, spherical, cage-type, and hybrid configurations. For instance, a J-type topology may employ a ring-shaped or toroidal particle body in which the interlocking openings are aligned along orthogonal or tri-axial directions. A T-type topology may employ a tetrahedral or multi-ring cluster geometry defining six nodes disposed about a central volume. A D-type topology may utilize a tetrahedral, Catalan tetrahedral, or icosahedral cage geometry configured for corner-to-corner interlocking. A C-type topology may employ a cuboctahedral or octahedral geometry providing six to eight interlocking sites arranged symmetrically about the particle center. In other embodiments, the particle body geometry can be toroidal, ring-clustered, or polyhedral-ring hybrid, each selected to provide the desired combination of rotational mobility and structural stiffness. In embodiments herein described, the particle body geometry defines the specific shape of the unit particles. Exemplary particle body geometries may include toroidal or ring shapes, as well as polygonal loops such as triangles, squares, or hexagons. The geometries can also comprise various polyhedral cages, including but not limited to a tetrahedron, octahedron, cube, cuboctahedron, truncated tetrahedron, icosahedron, or Catalan forms. Furthermore, the particle body geometry may be selected from hybrid ring-cluster or polyhedral-ring combinations, or specialized forms such as shell or sphere geometries that incorporate perforations or grooves to facilitate interlocking.

The term “structural member dimensions” as used herein refers to the measurable parameters defining the size and proportion of each structural member forming part of a particle body. Such parameters include, without limitation, the length, thickness, cross-sectional area, curvature, and spacing of the structural members relative to the overall geometry of the particle body. The structural member dimensions may be uniform or non-uniform and may be selected to achieve a desired balance between stiffness, flexibility, mass, and surface interaction within the architected material. Variations in structural member dimensions alter the local bending and load-bearing behavior of each particle and consequently influence the macroscopic mechanical properties of the material, including its effective modulus, energy-dissipation capacity, and transition between fluid-like and solid-like states. The relationship between the structural member dimensions and the clearances of the interlocking openings defines the mechanical tolerance and range of motion permitted at each node within the catenated lattice.

Exemplary structural member dimensions can vary in accordance with the selected geometry and topology. For instance, toroidal particle bodies may include annular walls or ribs having thickness-to-diameter ratios (d/D) between approximately 0.02 and 0.20, wherein thinner ratios yield greater rotational freedom and thicker ratios provide earlier jamming and higher stiffness. Polyhedral or cage-type particle bodies may include struts having lengths between approximately 0.1 and 10 millimeters and cross-sectional diameters between approximately 0.05 and 2 millimeters, with wall-thickness variations used to tune stiffness and energy absorption. The clearances between interlocking structural members and interlocking openings may be between approximately 1 and 10 percent of the characteristic feature size to allow controlled movement without disengagement. In certain embodiments, structural member dimensions are scaled uniformly or non-uniformly across the lattice to achieve hierarchical stiffness gradients or spatially programmable deformation within the architected material.

In embodiments herein described Catenation Topology, Particle Body Geometry, and Structural Member Dimensions provide structural variables to manipulate the material's internal kinematic degrees of freedom (DOFs (the ability of the discrete unit particles to move, rotate, and rearrange). By controlling the DOFs, the designer can precisely set the critical jamming strain, which is the quantitative threshold where DOFs are exhausted, the particles jam, and the material transitions from its fluid-like to its solid-like state.

In the context of this architected material, the wording “KinematicDegrees of Freedom”, or “DOFs”, indicate a collective property referring to the ability of the discrete, non-bonded unit particles to move, rotate, and rearrange relative to one another. This movement, which includes sliding at the nodes or reorienting the particle bodies, occurs without requiring elastic deformation of the structural members themselves. These DOFs are an inherent function of the structure's design and are “operated” or controlled by the structural variables. Catenation topology determines the type of collective motion allowed, such as shear-based rearrangement versus compression-based scissoring. Particle body geometry determines the efficiency of packing; for instance, open toroidal rings allow more movement than dense polyhedral cages. Structural member dimensions, such as the d/D ratio, determine the amount of free space or clearance at the nodes, directly controlling the range of motion before particles make contact.

A structure with high DOFs has ample free space, allowing its particles to rearrange extensively, which results in the fluid-like regime. A structure with zero or exhausted DOFs is one where the particles are in contact and can no longer rearrange, forcing it into the solid-like regime. The DOFs are not measured as a single number but are detected by observing their effect on the material's bulk properties. For example, in a state of high DOFs, the material exhibits a near-zero shear modulus. This is detected as a “plateau” in a shear stress-strain test, where shear strain increases with no corresponding increase in shear stress. In dynamic oscillatory tests, a state of high DOFs is also characterized by low values for both the storage modulus, G′, and the loss modulus, G″. This state is also observed as a shear-thinning response, where G′, G″, and complex viscosity decrease as the torsional strain or angular frequency increases. In a digital twin simulation, DOFs are high when the mean contact number between particles is low. As DOFs are exhausted, this mean contact number value increases, indicating particles are making contact and jamming.

The term “digital twin” as used herein refers to a computer-based or virtual representation of a physical object, system, or process, which is dynamically linked to the corresponding physical counterpart through data exchange. A digital twin may incorporate mathematical models, geometric or topological representations, and real-time or recorded sensor data describing the physical state, configuration, or operating conditions of the associated object. The digital twin may simulate or predict the physical object's performance, behavior, or response under given conditions and may be continuously updated as new data are obtained from the physical system. In some embodiments, the digital twin provides a bidirectional feedback interface between the computational environment and the physical object, allowing measurements or control commands derived from the digital twin to be used to monitor, adjust, or optimize the physical system. The digital twin may represent a single component, such as an individual particle body, or a complex assembly, such as an entire architected material structure, and may include multiple hierarchical submodels corresponding to different levels of scale or functionality.

The term “model” refers to a mathematical, analytical, or data-driven representation of an object, process, or system. A model may take the form of numerical equations, finite-element meshes, statistical regressions, or neural-network architectures that describe relationships among variables representing physical, chemical, or mechanical phenomena. In the context of the digital twin, the model defines the computational framework used to simulate or predict the behavior of the physical counterpart, including its geometry, topology, boundary conditions, and material properties. Models may be deterministic, where outputs are fully defined by input parameters, or probabilistic, where uncertainty or stochastic variability is included. A model may further include parametric definitions of the particle body geometry, structural member dimensions, and catenation topology of an architected material, allowing for virtual testing, optimization, and control of its mechanical or functional performance. In some embodiments, the model is trained, calibrated, or validated against experimental or sensor-derived data from the physical system to ensure predictive accuracy.

In embodiments herein described the DOF can be used to identify the Critical Jamming Strain which is primary result effective variable for the mechanical behavior of the architected material herein described.

The wording “Critical Jamming Strain”, as used herein refers to a critical compressive strain (Ex) or a critical shear jamming strain (Y's), and indicates the specific, measurable threshold of deformation at which the structure's KinematicDegrees of Freedom are exhausted. It is the precise event that marks the transition from the fluid-like regime to the solid-like regime. Below this strain value, the particles are free to rearrange. At and above this strain value, the particles are “jammed” against each other, preventing further rearrangement. Any additional deformation requires the elastic deformation, such as bending or stretching, of the particles' structural members. This causes the material to stiffen and behave as a solid.

In embodiments, herein described modification of the configuration of the topology and geometry of the unit cells and the material in its entirety, can be selected to modify the critical jamming strain as tunable parameter of the architected material. A designer can “program” this value by selecting the catenation topology, particle geometry, and structural member dimensions, herein described. The critical jamming strain is detected by identifying the exact point of transition in mechanical tests. In a quasi-static compression or shear test, the critical jamming strain is the strain value at which the stress-strain curve departs from the near-zero stress plateau and begins to rise in a nonlinear, elastic manner. In an oscillatory amplitude sweep, the critical jamming strain is the torsional strain value at which the storage modulus, G′, loss modulus, G″, and complex viscosity, η*, reach their minimum values and begin to increase. This inflection point marks the onset of shear-thickening, which is the signature of the jamming transition.

In particular in embodiments herein described modification of the structural variables in order to tune the resulting material properties can be performed by selecting the Catenation Topology (The High-Level Network Pattern), and on this basis the Particle Body Geometry (The Particle Shape) and then 3. Structural Member Dimensions (Thickness and Clearance).

In particular, in embodiments herein described, the 3D crystalline network topology (e.g., J, T, D, C) is the primary structural variable. It operates by dictating the coordination and type of motion allowed between particles. Different topologies create different kinematic mechanisms, programming the material to be flexible in one mode (e.g., shear) but stiff in another (e.g., compression).

For example, a J-4-ring topology is structured to allow for a coordinated rearrangement of particles under shear load. This design creates a high number of DOFs for shearing motion. This, in turn, results in a high critical shear jamming strain (Ys), making the material highly deformable and fluid-like under shear.

In a further example, a T-6-ring topology is structured to utilize a “scissor” mechanism between particles under compressive load. This design creates a high number of DOFs for compressive motion. This results in a high critical compressive jamming strain (ex), making the material highly effective at absorbing energy under compression.

By selecting the catenation topology, a designer can program an anisotropic jamming response. In the sense of the disclosure, an anisotropic response refers to the material's properties not being the same in all directions. This is an intrinsic characteristic inherited from the material's “crystalline network-based designs”. The resulting mechanical behavior is highly anisotropic and is dependent on the load-to-Symmetry axis alignment. This anisotropy is demonstrated, for example, by the ability to program the material to be flexible in one mode (e.g., shear for a J-4-ring topology) but stiff in another, or flexible in a different mode (e.g., compression for a T-6-ring). It is also physically manifested in “gravity relaxation outlines,” where a spherical structure will settle into different final shapes depending on which of its crystallographic axes (<100>, <110>, or <111>) is oriented vertically. Furthermore, this anisotropy affects the material's “shear symmetry,” resulting in a “pronounced anisotropic” shear response that varies as the sample is rotated relative to the loading direction as will be understood by a skilled person.

In embodiments herein described, the resulting mechanical behavior of the structure is programmed by a specific interplay between the structural variables and the applied forces.

In particular, in embodiments herein described for a given topology, the choice of the particle body geometry is the secondary structural variable. It operates by controlling the local packing efficiency and the ease with which particles can move past one another. This variable tunes the overall stiffness and flexibility of the material.

For example, a toroidal (ring) geometry (e.g., J-4-ring) is open and curved, which maximizes the free space between particles. This structure results in high DOFs and a correspondingly high critical jamming strain.

Additionally, in a further example a polyhedral (cage) geometry (e.g., J-4-OCT) has flat faces and distinct corners. These features pack more tightly and make contact (jam) with less relative movement. This structure results in low DOFs and a correspondingly low critical jamming strain, making the material stiffer. More open geometries (like toroidal and loop) result in higher DOFs and higher critical strain levels, whereas the cage-like polyhedral designs show earlier jamming and lower critical strain levels for the same topology.

Accordingly, for embodiments herein described, for a given topology, the choice of the particle body geometry is the secondary structural variable. It operates by controlling the local packing efficiency and the ease with which particles can move past one another. This variable tunes the overall stiffness and flexibility of the material. For example, a polyhedral (cage) geometry, such as a J-4-OCT, has flat faces and distinct corners. These features cause the particles to pack more tightly and make contact, or jam, with less relative movement. This structure consequently results in low kinematic degrees of freedom (DOFs) and a correspondingly low critical jamming strain, making the material stiffer. More generally, for the same given topology, more open geometries, such as toroidal and loop shapes, result in higher DOFs and correspondingly higher critical strain levels. In contrast, cage-like polyhedral designs show earlier jamming and lower critical strain levels.

In embodiments, herein described, the Structural Member Dimensions (Thickness and Clearance) is the most direct variable for “fine-tuning” the material's response. It operates by directly controlling the amount of free space, or “clearance,” between interlocked particles at the nodes. This is quantifiable as the ratio of the structural member thickness (d) to the interlocking opening diameter (D), or (d/D).

Accordingly, the critical jamming strain is inversely related to the d/D ratio. In particular using thinner structural members (a low d/D ratio, e.g., 0.14) creates more empty space and larger clearances. This increases the DOFs and raises the critical jamming strain, making the material more fluid-like and flexible. Conversely, using thicker structural members (a high d/D ratio, e.g., 0.275) reduces empty space and clearances. This decreases the DOFs and lowers the critical jamming strain, causing the material to jam sooner and behave as a stiffer solid. While these numeric values are based on exemplars, this inverse trend is expected to be applicable to the structure of the materials of the disclosure. Additionally, the Solidity Ratio (SR) of the structure also influences its behavior; a higher SR results in a higher baseline stiffness and causes the material to jam earlier.

Accordingly, in embodiments herein described, by selecting and combining these three structural variables, a designer can precisely program the critical jamming strain, and therefore the entire dual-behavior response, of the architected material, as will be understood by a skilled person upon reading of the present disclosure.

In some embodiments, a structure is described, comprising: a plurality of unit particles, each unit particle comprising a particle body formed from one or more structural members and defining one or more interlocking openings at one or more nodes.

In the structure of the present disclosure, the plurality of unit particles are interconnected to form a three-dimensional network patterned after a crystalline network topology and comprising inter-layer linkages.

In the structure of the present disclosure, the interconnection is a mechanical interlock formed by an interlocking structural member of one unit particle passing through an interlocking opening of an adjacent unit particle at a node, such that the particle bodies are topologically intertwined and cannot be separated without being cut or broken. This interconnection provides three-dimensional cohesion arising from mechanical interlocks at nodes, including inter-layer linkages. The adjacent particle bodies are not bonded (e.g., no welded or adhesive joints) and are topologically inseparable absent cutting or fracture.

The term “adjacent bodies,” as used herein, refers to two or more unit particles or particle bodies that are positioned in proximity to one another within the three-dimensional network. In the context of the present disclosure, “adjacent” signifies that the particle bodies are immediate neighbors in the patterned structure, configured for mechanical interconnection. In some embodiments, a first particle body is adjacent to a second particle body when an interlocking structural member of the first particle body is positioned to pass through a corresponding interlocking opening of the second particle body at a node. This adjacency facilitates the formation of the mechanical interlock that provides cohesion to the structure. Adjacent bodies, therefore, are those particle bodies that are topologically intertwined or configured to become so, and are distinct from particle bodies that are spatially remote or not configured for direct interconnection within the crystalline network topology.

The term “mechanical interlock,” as used herein, refers to the specific interconnection formed between adjacent unit particles within the structure. This interconnection is formed by an interlocking structural member of one unit particle passing through an interlocking opening of an adjacent unit particle at a node such that the particles cannot be completely separated without cutting one or more of the particles. The mechanical interlock is distinct from an adhesive or welded joint, as the adjacent particle bodies are not bonded. Instead, a mechanical interconnection provides three-dimensional cohesion to the structure, with said cohesion arising from the mechanical interlocks at nodes and comprising inter-layer linkages. This arrangement results in the particle bodies being topologically inseparable absent cutting or fracture as they form the three-dimensional network patterned after a crystalline network topology.

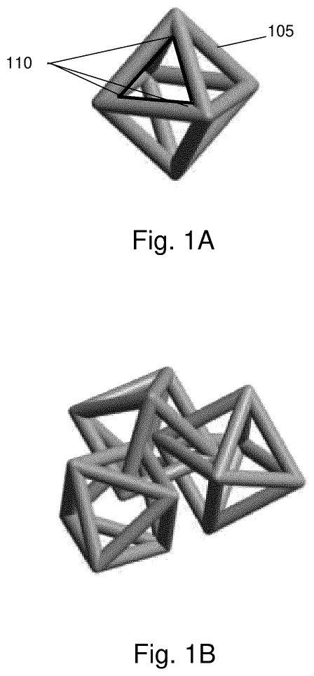

Accordingly, in an exemplary embodiment herein described, the structure is composed of unit particles having lattice-like (hollow) geometries, in that they are two- or three-dimensional rigid bodies that form one or more “loops” in their body. An example is shown in FIG. 1A, with an octahedron shape for this example. The rigid trusses (105) make up the shape, with triangular “loops” (110) between three connected trusses. Other geometries (cubes, pentagonal trapezohedrons, circles, spheres, etc.) are also possible, including irregular geometric shapes, so long as they contain at least one “loop”. The trusses can be straight or curved or bent or irregular. The number of “loops” for a given geometric shape can vary, depending on how many trusses are used to make the shape.

The purpose of the opening, in the exemplary unit particles of FIG. 1A is to allow the unit particles to interlock with one another, as shown in the example of FIG. 1B. The interlocking of the unit particles allows the combined unit particles to jam together under applied pressure, while remaining pliable when pressure is not applied.

Accordingly, in embodiments herein described, the structure is formed from interlocking components, which can be generalized into a “Host” particle and a “Guest” particle. The “Host” Particle is the unit that defines an opening or “eyelet.” Exemplary “Host” particles anchored to tested data include a Toroidal/planar ring, a Polygonal loop (such as a J-4-square or T-6-HEX), or a Polyhedral cage with apertures (such as a J-4-OCT or D-4-TET). Other general examples may include a slotted sheet or a perforated plate. The “Guest” Particle is the unit with a projecting member that passes through the opening of the “Host” Particle. Exemplary “Guest” particles anchored to tested data include another ring or loop (in ring-ring catenation), a cage strut or vertex (in corner/edge interlocks), or a cluster of loops. Other general examples can include a tab on a sheet or a T-shaped beam. The reference to “host” and “guest” are relative terms, as each “host” is also a “guest” to its “guests” (which each act as its respective “host” as will be understood by a skilled person.

In embodiments herein described, the structure is further configured for actuation by a selected trigger. This configuration can comprise a structural scale, a constituent material, or a coating applied to the plurality of unit particles that allow at least one transition from different conversion states as will be understood by a skilled person upon reading of the present disclosure.

In particular, in embodiments herein described, the structure can is configured to exhibit several programmable “conversion states,” which are transitions from a first state to a second state in response to a trigger or condition. These transitions define the unique functional behaviors of the material.

In some embodiments, a primary conversion state is the Jamming Transition, which defines the material's dual mechanical response. The material transitions from a first “fluid-like regime” to a second “solid-like regime.” The first state, or fluid-like regime, occurs at strains below a critical jamming strain and is characterized by a near-zero shear modulus and shear-thinning behavior, as the unit particles possess kinematic degrees of freedom (DOFs) allowing them to rearrange. The second state, or solid-like regime, occurs when the applied strain exceeds the critical jamming strain, exhausting the DOFs and “jamming” the particles. This solid-like state is characterized by a nonlinear stress-strain response, significant strain-stiffening, and shear-thickening behavior.

In some embodiments, another conversion state is Actuated Shape-Morphing (ASM), which is typically observed in micro-scale embodiments coated with a conductive material. The structure transitions from a first “compact” or uncharged state, where it may be collapsed, to a second “deployed” or charged state. This transition is triggered by an electrostatic field, which causes mutual electrostatic repulsion between the unit particles, forcing the structure to expand. This state change is rapid and reversible upon neutralizing the electrostatic charge.

In some embodiments, a further conversion state is Multi-Stable Configurations, which can be exhibited by structures with specific topologies, such as a J-4-square topology. This involves a transition between a first stable configuration, such as an “expanded” or stiff state, and a second stable configuration, such as a “collapsed” or flexible state.

In some embodiments, a further conversion state relates to Preconditioning under cyclic loading. The structure transitions from a first “initial” or “virgin” state, characterized by high energy dissipation and a large hysteresis loop, to a second “steady-state” or “preconditioned” state after a number of loading cycles. This second state is characterized by lower energy dissipation and a smaller, stable hysteresis.

In some embodiments, an additional conversion state is Structural Relaxation post-fabrication. The structure transitions from its first “as—designed” or ordered state, which corresponds to the ideal periodic microstructure in a digital file, to a second “as-relaxed” or disordered state. This second state is its functional, irregular state after settling under gravity or other external forces.

In embodiments herein described, the resulting mechanical behavior of the structure, particularly its transition between conversion states, is governed by a set of programmable results-effective variables. These variables function as the Primary Structural Variables and secondary Geometric Tuning Parameters (or Dimensional Knobs) that allow a designer to precisely set the kinematic degrees of freedom (DOFs) and, consequently, the critical jamming strain of the material. These variables are broadly categorized as Topology, Geometry, and Dimensional Knobs.

The Topology is the primary structural variable and refers to the high-level network pattern of connectivity. This includes the Catenation Number (CN), which defines the number of adjacent particles each unit particle is interlocked with, such as a CN of 4 or 6. The topology also includes the Catenation Symmetry, which describes the spatial arrangement of these interlocks, such as a planar, tetrahedral (Td), or octahedral (Oh) symmetry. The resulting mechanical behavior is highly anisotropic and depends on the load-to-symmetry axis alignment. For example, a structure with a planar symmetry (e.g., J-4-ring) can be configured to provide high flexibility and a high critical shear jamming strain when loaded in-plane, while a structure with planar hexagonal, tetrahedral or octahedral symmetry (e.g., T-6-ring) may be configured to provide high compressive flexibility via a scissor-like mechanism when loaded under compression. In embodiments herein described, the Catenation Topology defines the three-dimensional pattern of mechanical connectivity. This topology may be patterned after a three-dimensional crystalline lattice, such as a diamond, cubic, or tetrahedral network. Exemplary catenation topologies may be derived from crystalline or graph-based networks and may include, but are not limited to, J-type, T-type, S-type, D-type, or C-type topologies. These may correspond to Reticular Chemistry Structure Resource (RCSR) symbols such as dia, pcu, pcu-b, bcc/bcu, fcc (octet), nbo-b, pbz/hxg, or srs. Specific proven topologies include J-4-ring, J-4-square (which can be multi-stable, exhibiting ‘L’ and ‘S’ states), T-6-ring, T-6-HEX, J-4-OCT, S-6/2-OCT, D-4-TET, and C-6-TT.

The Geometry is a secondary structural variable that defines the specific shape of the unit particles. The particle body geometry controls the local DOFs and packing efficiency of the particles. For example, a toroidal or ring-shaped particle geometry is open and curved, which maximizes the free space between particles, allows for greater rotational and translational movement, and thus provides higher local DOFs. In contrast, a polyhedral cage geometry, having flat faces and distinct corners, packs more tightly and makes contact with adjacent particles with less relative movement. This polyhedral geometry thereby reduces the local DOFs and results in a stiffer material that jams at an earlier strain.

In embodiments herein described, the Particle Body Geometry this refers to the three-dimensional form, shape, and symmetry of each discrete element. Exemplary particle body geometries corresponding to the topologies of architected materials in the sense of the disclosure comprise toroidal/rings, polygonal loops (such as Square or Hexagon), polyhedral cages (such as Tetrahedron (TET), Catalan Tetrahedra (CT), Icosahedra (ICO), Cube, Octahedron (OCT), Cuboctahedron (CO), or Truncated Tetrahedron (TT)), shell-type, spherical, and hybrids of two or more geometries.

The Geometric Tuning Parameters (or Dimensional Knobs) are quantitative, fine-tuning variables that directly control the clearances and mass distribution of the structure. These parameters include the d/D ratio, which is the ratio of the structural member thickness (d) to the interlocking opening size (D). This ratio is inversely related to the critical jamming strain; a low d/D ratio (e.g., 0.14) creates larger clearances, increases DOFs, and thus raises the critical jamming strain, while a high d/D ratio (e.g., 0.275) reduces clearances and lowers the critical jamming strain.

Accordingly, in embodiments, herein described, the structural member dimensions are the quantitative, fine-tuning variables for “fine-tuning” the material's response. This is quantifiable as the ratio of the structural member thickness (d) to the interlocking opening diameter (D), or (d/D). The tested range for ring exemplars is approximately 0.14 to 0.275, while a general described range for toroidal bodies can be approximately 0.02 to 0.20. A low d/D ratio (e.g., ≈0.14) results in higher critical jamming strains. A high d/D ratio (e.g., ≈0.275) results in lower critical jamming strains.

A more general form of this parameter is the Clearance Ratio (CR), calculated as (Opening Size-Member Size)/Opening Size. This ratio quantifies the normalized “wiggle room” at a node, and it has a monotonically increasing relationship with the DOFs (a higher CR provides higher DOFs). Another dimensional parameter is the Solidity Ratio (SR), defined as the solid volume divided by the unit cell volume. The SR has a monotonically increasing relationship with stiffness; a higher SR results in a higher baseline stiffness and earlier jamming.

An additional variable is the Interlock Aspect Ratio (IAR), defined as the length-to-width of the projecting interlocking feature, describes the qualitative nature of the interlock. A high IAR (a long, thin feature) facilitates rod-like sliding, while a low IAR (a short, stubby feature) acts as a key-like constraint that limits the axes of motion.

In embodiments herein described, specific conversion states are achieved by selecting particular combinations of Primary Structural Variables and Geometric Tuning Parameters (or Dimensional Knobs). These combinations provide “recipes” for engineering a structure with a predefined mechanical behavior.

In a specific embodiment, a structure is configured for high compressive flexibility to achieve a Jamming Transition optimized for shock absorption. This is achieved by selecting a T-type topology, such as a T-6-ring, having a Catenation Number (CN) of approximately 6 and a planar hexagonal, tetrahedral or octahedral symmetry. The particle body geometry is a toroidal ring or hexagonal loop, which enables a “scissor” mechanism under compressive loading. The dimensional knobs are tuned for a low d/D ratio, defined in this context as a d/D ratio in a range of approximately 0.14 to 0.17, which corresponds to a high Clearance Ratio. This specific combination of variables results in a structure configured to exhibit a high critical compressive jamming strain (Ex), defined in this context as a strain in a range of approximately 10% to 28%.

In another specific embodiment, a structure is configured for high shear flexibility to achieve a Jamming Transition optimized for morphing. This is achieved by selecting a J-type topology, such as a J-4-ring, having a Catenation Number (CN) of approximately 4 and a planar symmetry. The particle body geometry is a toroidal ring. This topology and geometry are configured to allow for the coordinated rearrangement of particles when under in-plane shear. The dimensional knobs, such as the d/D ratio, are selected in a range of approximately 0.14 to 0.275. A low d/D ratio (corresponding to a high Clearance Ratio) within this range results in a high critical shear jamming strain (Y). In this context, a high critical shear jamming strain is defined as a strain in a range of approximately 30% to 70%.

In a further specific embodiment, a structure is configured for the Actuated Shape-Morphing conversion state. This is achieved by selecting a high-DOF primary structural variable, such as a J-type topology (e.g., J-4-ring), and fabricating it at the micro-scale. As used herein, micro-scale is defined as a structure having a sample side length of less than or equal to 1 mm. The dimensional knobs are tuned for a low Solidity Ratio (SR) to reduce particle mass and a high Clearance Ratio (CR) to allow for deployment. Furthermore, the structure includes a conductive material coating, such as a coating of approximately 300 nm of copper. This specific set of variables results in a structure that, when an electrostatic charge is applied, transitions from a first “compact” state to a second “deployed” state in a rapid time. In this context, rapid time is defined as a transition time of less than 0.1 seconds.

In yet another specific embodiment, a structure is configured for the Multi-Stable Configurations conversion state. This is achieved by selecting a J-4-square topology as the primary structural variable, which utilizes a Catenation Number of 4 in a planar layout with a square polygonal loop particle body geometry. This specific combination enables an auxetic “rotating squares” mechanism. The resulting structure can be “trained” by cyclic loading to transition between a first “expanded” or stiff ‘L’ configuration, characterized by a relaxed volume fraction of approximately 15.67%, and a second “collapsed” or flexible ‘S’ configuration, characterized by a relaxed volume fraction of approximately 35.23%.

In addition to the specific conversion states exemplified herein, it will be understood by a person skilled in the art that the architected material structure of the present disclosure provides a general framework for engineering a wide variety of mechanical behaviors and additional conversion states. The fundamental relationships between the Primary Structural Variables-such as Catenation Topology, Catenation Number, and Catenation Symmetry- and the Geometric Tuning Parameters—such as Clearance Ratio, Solidity Ratio, and d/D ratio-provide a robust system for precisely programming the kinematicDegrees of Freedom and resulting mechanical response of the structure. By selecting and combining these variables in manners not explicitly detailed, one can tune the material to achieve further, novel transitions between first and second states, tailoring the material's function to different triggers or mechanical environments.

In embodiments herein described, the structures exhibit orientation-dependent anisotropy, a property derived from their crystalline network-based designs. This anisotropy is evident in the material's response to both static and dynamic forces. For example, in a static state, the structure's final relaxed shape under gravity is dependent on its initial crystallographic orientation; spherical J-4-ring samples, for instance, will settle into different non-spherical outlines when oriented along their <100>, <110>, or <111>axes. This anisotropy can also manifest mechanically, such as in the material's “shear symmetry,” where the shear response is pronouncedly anisotropic and varies depending on the rotation of the sample's structural axes relative to the applied shear direction.

Accordingly, the structure herein described can be configured for actuation by one or more selected triggers. This configuration is achieved by selecting specific Primary Structural Variables and Geometric Tuning Parameters, such as the structural scale and constituent material, or by applying additional features, such as a coating to the structural members or particle bodies. For example, to configure the structure for actuation by an electrostatic field, the structure is fabricated at a micro-scale, defined herein as a sample side length of less than or equal to 1 mm. Furthermore, the plurality of unit particles are fabricated from or, more preferably, coated with a conductive material, such as a coating of approximately 300 nm of copper, to allow the particles to hold a charge.

In some embodiments, a structure according to the present disclosure can be obtained in outcome of a method for engineering a structure of the present disclosure. The method comprises: determining a particle body geometry, the particle body geometry being formed from one or more structural members and defining one or more interlocking openings at one or more nodes; determining a 3D crystalline network topology and determining one or more structural member dimensions, a constituent material, and a structural scale.

The method for engineering a structure in accordance with the present disclosure further comprises selecting the particle body geometry and the 3D crystalline network topology the one or more structural member dimensions, the constituent material, and the structural scale to achieve a target conversion state activatable by a selected trigger; and forming a digital representation of a structure by creating a plurality of unit particles with the particle body geometry, the unit particles being mechanically interlocked with each other in the 3D crystalline network topology by an interlocking structural member of one unit particle passing through an interlocking opening of an adjacent unit particle.

In embodiments herein described, the method for engineering a structure can be performed as a multi-step design process for creating a digital representation of the architected material.

In those embodiments determining a 3D crystalline network topology, which functions as a Primary Structural Variable, can be performed by selecting a topology template based on a desired target mechanical behavior, such as selecting a J-type topology for high shear flexibility

$$( {γ}_{s}^{*} )$$

or a T-type topology to achieve high compressive flexibility

$$( {ϵ}_{c}^{*} ) .$$

For example, a topology that allows for a high degree of coordinated particle rearrangement where particles can slide, rotate, or reorganize collectively without immediate jamming would likely also exhibit high shear flexibility. Likewise, high compressive flexibility is a programmable characteristic that can be achieved by selecting a topology that enables a specific type of coordinated particle rearrangement under compressive loads which reflect the amount the material can deform before its particles “jam” and it transitions from a fluid-like to a solid-like state—for example, a “scissor” like kinematic mechanism in the topology created by nodes comprising cluster of spatially arranged planar particles, rather than a single rigid polyhedral node.

In those embodiments the method further comprises determining a particle body geometry, which can be performed by parametrizing the unit particle geometry, which defines the specific shape of the particle body (e.g., toroidal, polygonal loop, or polyhedral cage) and the specific geometry and location of the node openings on said particle body. Following the topology selection, the method comprises parametrizing the unit particle geometry.

In those embodiments the method can also comprises determining one or more structural member dimensions, which can be performed by assigning the specific Geometric Tuning Parameters, or Dimensional Knobs. This includes assigning quantitative values for the structural member dimensions, such as the thickness ‘d’ and opening size ‘D’, and thereby defining the resulting ratios, such as the d/D ratio, the Clearance Ratio (CR), and the Solidity Ratio (SR).

The method then comprises selecting the determined topology, geometry, and structural member dimensions to achieve a target critical jamming strain. This selection may be an iterative process. In some embodiments, this step includes performing an optional simulation by using the digital representation as a digital twin and using a contact-aware simulation module (e.g., LS-DEM) to predict the critical jamming strain

$$( {γ}_{s}^{*} or {ϵ}_{c}^{*} ) .$$

Based on the simulation results, the method may comprise iterating on the design by modifying the assigned dimensional parameters, such as the d/D ratio or CR, and re-simulating until the predicted jamming strain matches the target.

Additionally, the method comprises forming a digital representation of a structure. This step can be performed by building the full, interlocked lattice in a Computer-Aided Design (CAD) environment, which includes checking for unintended interference while verifying the intended mechanical interlocks and clearances. This step is completed by exporting a build file (e.g., an .stl file) and locking the design for additive manufacturing.

In some embodiments, the method further comprises an optional simulation step. In this step, the digital representation is used as a digital twin, and a contact-aware simulation module, such as a Level Set-Discrete Element Method (LS-DEM) module, is used to predict the critical jamming strain

$$( {γ}_{s}^{*} or {ϵ}_{c}^{*} ) .$$

Based on the simulation results, the method may further comprise iterating on the design to meet the target jamming strain. This iteration involves modifying the assigned dimensional parameters, such as the d/D ratio or CR, and re-simulating. Once the predicted jamming strain matches the target, the digital representation is locked and finalized for additive manufacturing.

In a preferred embodiment, the method for engineering a structure follows a specific design flow. The flow begins with the selection of a catenation topology, which is chosen to target a. specific primary mechanical response, such as a target critical shear jamming strain

$$( {γ}_{s}^{*} )$$

or a target critical compressive jamming strain

$$( {ϵ}_{c}^{*} ) .$$

For example, a J-4 planar topology may be selected to achieve high shear flexibility, while a T-6 topology may be selected to achieve a compressive “scissor” motion for high compressive flexibility. After selecting the topology, a particle geometry is chosen, such as a toroidal versus a polyhedral geometry, to adjust the local kinematic degrees of freedom (DOFs). Next, the dimensional knobs, or Geometric Tuning Parameters, are picked. This includes selecting a specific thickness-to-diameter (d/D) ratio, such as a ratio within the demonstrated range of approximately 0.14 to 0.275 for ring exemplars, to fine-tune the DOFs and resulting critical jamming strain. Once these variables are set, the digital representation of the structure is generated. Finally, the method may optionally include a step to simulate the digital representation to predict the critical jamming strain before proceeding with fabrication.

Accordingly, in embodiments herein described, the method for engineering a structure can be performed as a multi-step design process using results effective variables operated in accordance with the present disclosure.

The first variable is the Crystalline/network topology, which defines the high-level pattern of connectivity and is often described using RCSR-style (Reticular Chemistry Structure Resource) symbols. Examples include dia (diamond), pcu (primitive cubic, including pcu-b), bcc/bcu (body-centered cubic), fcc (face-centered cubic, or octet), nbo-b (an analog for the J-type topology), pbz/hxg (an analog for the T-type topology), and srs. This framework also includes related 3D periodic networks as well as hierarchical or hybrid combinations. Other common topologies used in architected materials include structures based on Triply Periodic Minimal Surfaces (TPMS), such as the Gyroid, Schwarz P, and Schwarz D surfaces.

The second variable is the Particle geometry, which defines the specific shape of the unit particles and controls the local kinematic degrees of freedom. There are a wide range of geometries, including simple toroidal/rings and polygonal loops like triangles, squares, or hexagons. This category also includes numerous polyhedral cages such as the tetrahedron (TET), octahedron (OCT), cube, cuboctahedron (CO), truncated tetrahedron (TT), icosahedron (ICO), and Catalan forms.

Finally, the geometries can also be specialized forms like hybrid ring-cluster/polyhedral-ring combinations or shell/sphere structures that possess perforations or grooves to enable interlocking. Other geometries common in the broader field of metamaterials include the dodecahedron and rhombic dodecahedron, as well as functional unit cells like auxetic (re-entrant) or chiral structures.

Accordingly, in some embodiments, a structure according to the present disclosure can be obtained in outcome of a method for engineering a structure of the present disclosure. This method can be performed as a multi-step design process for creating a digital representation of the architected material.

The method comprises determining a 3D crystalline network topology, which functions as a Primary Structural Variable. This may be done by selecting a topology template based on a desired target mechanical behavior, such as selecting a J-type topology for high shear flexibility (γs*) or a T-type topology for high compressive flexibility (ϵc*).

The method further comprises determining a particle body geometry, for example, by parametrizing the unit particle geometry, which defines the specific shape of the particle body (e.g., toroidal or polyhedral cage) and the specific geometry and location of the node openings on said particle body.

Following the topology and geometry selection, the method comprises determining one or more structural member dimensions, for example, by assigning the specific Geometric Tuning Parameters. This includes assigning quantitative values for the structural member dimensions, such as the thickness ‘d’ and opening size ‘D’, and thereby defining the resulting ratios, such as the d/D ratio, the Clearance Ratio (CR), and the Solidity Ratio (SR).

The method then comprises selecting the determined topology, geometry, and structural member dimensions, and further selecting a constituent material and a structural scale, to achieve a target conversion state activatable by a selected trigger. For example, if the selected trigger is a mechanical force, the target conversion state may be a Jamming Transition, and the selection is made to achieve a target critical jamming strain (γs* or ϵc*).

In some embodiments, this selection is an iterative process. This may optionally include a simulation step, where the digital representation is used as a digital twin and a contact-aware simulation module (e.g., LS-DEM) is used to predict the critical jamming strain (γs* or ϵc*). Based on the simulation results, the method may comprise iterating on the design by modifying the assigned dimensional parameters, such as the d/D ratio or CR, and re-simulating until the predicted jamming strain matches the target.

Finally, the method comprises forming a digital representation of a structure. This step can be performed by building the full, interlocked lattice in a Computer-Aided Design (CAD) environment, which includes checking for unintended interference while verifying the intended mechanical interlocks and clearances at the nodes. This step is completed by exporting a build file (e.g., an .stl file) and generating the corresponding machine toolpaths and process parameters (e.g., slicing for STL, exposure paths, laser power, scan speed) a locking the design for additive manufacturing.

In embodiments herein described, the method for engineering a structure of the disclosure can be performed with a corresponding system for engineering a structure. The system comprises a data storage module configured to store a library of 3D crystalline network topologies and a library of particle body geometries, which may be referred to as a topology/geometry library. The system further comprises a modeling engine, such as a parametric modeling engine, configured to generate a digital representation of a structure by combining a selected topology, a selected geometry, and one or more selected structural member dimension.

A simulation module can also be included, configured to apply virtual forces to the digital representation to compute a predicted critical jamming strain. This module may be a contact-aware simulation module, such as a DEM/LS-DEM or rigid multibody with non-smooth contact module. Finally, the system comprises a user interface configured to receive user selections for the topology, geometry, and structural member dimensions, and to display the predicted critical jamming strain. This allows a user for selecting variables and reporting predicted

$${γ}_{s}^{*} / {ϵ}_{c}^{*} .$$

In some embodiments, the method for engineering a structure can be performed with a system for engineering a structure. The system for engineering a structure provides a tangible, computer-implemented manufacturing workflow that links digital design, simulation, and physical fabrication in a closed-loop process. In an example embodiment, the system comprises a user interface for receiving user selections for a 3D crystalline network topology, a particle body geometry, and one or more structural member dimensions, and a contact-aware simulation module, such as a DEM/LS-DEM module, configured to compute and report a predicted critical jamming strain, such as

$${γ}_{s}^{*} or {ϵ}_{c}^{*} .$$

This computation and iterative design phase can be directly tied to a physical output, as the system's parametric modeling engine can be configured to generate a digital representation and/or export a concrete build file, for example, an .stl file, based on the finalized and validated parameters. This build file is not merely an abstract representation but constitutes a set of tangible instructions for a controller communicatively coupled to a manufacturing device. These instructions define specific fabrication parameters, such as the toolpaths and exposure paths for a two-photon lithography process, or the laser power and scan speed for a Selective Laser Sintering (SLS) or Selective Laser Melting (SLM) process. Following the fabrication and any required post-processing, such as a solvent clean or plasma etch to free interlocks, the structure may be further processed by applying a conductive coating, for instance, by sputtering copper on all or part of the structure. The process loop is then closed as the resulting physical structure is measured, for example, via mechanical couponing using compression, shear, or rheology tests, and this new experimental data can be fed back to the system to be used to update, calibrate, or validate the digital twin's predictive model, thereby refining the accuracy of future computations and subsequent fabrication cycles.

Embodiments of the disclosure encompass method and a system for actuating a structure of the present disclosure by triggering the conversation from an initial first state to an actuated second state.

The method for actuating a structure of the present disclosure comprises: providing a structure of the present disclosure in an initial first state; applying an electrostatic charge to the plurality of unit particles; and causing the structure to convert expand from the initial first state to a second actuated state due to electrostatic repulsion between the unit particles, wherein the conversion is reversible upon neutralizing the electrostatic charge.

In some embodiments, the transition from an initial first state to a second actuated state can include multiple state transitions (e.g. from fluid-like to semi-fluid-like to semi-solid-like to solid-like). The transition can also be continuous with a gradient of state properties (e.g. from fluid-like, transitioning smoothly to solid-like by increasing solid-like behavior as the trigger force increases).

In some embodiments herein described, the method for actuating a structure of the present disclosure can comprise providing a micro-scale structure that has been fabricated and released, such that its internal kinematic degrees of freedom are active. The method further comprises depositing a conductive coating onto the plurality of unit particles, for example, by sputtering a metal. The conductively-coated structure is then mounted on a substrate, such as an insulating substrate or an ITO-coated glass substrate, and an electrical connection is established to the conductive coating. The method proceeds by applying an electrical potential to the structure, which causes the unit particles to accumulate an electrostatic charge. This charge induces mutual electrostatic repulsion between the particles, causing the structure to convert from a first “compact” state to a second “deployed” state, a transition which can be observed. This conversion is reversible, and the method further comprises neutralizing the charge (e.g., by grounding) to cause the structure to revert from the deployed state back to the compact state. This process of charging and neutralizing to actuate the structure may be repeated cyclically.

The system for actuating a structure of the present disclosure is an electrostatic actuator system, comprising: one or more structures according to the present disclosure; and an electrical voltage source communicatively coupled to the conductive material of the plurality of unit particles, wherein the electrical voltage source is configured to apply an electrostatic charge to the unit particles, causing the structure to reversibly change shape.

In some embodiments herein described, the system can comprises one or more structures as herein described, which are preferably micro-scale and include a conductive material or coating. The system can further comprises a voltage source configured to apply an electrostatic charge, such as a Van de Graaff generator or a high-voltage (HV) DC supply. A conductive pathway can be included to communicatively couple the voltage source to the conductive material of the unit particles. This pathway can comprise conductive leads and fixtures, as well as a substrate on which the structure is mounted, such as an ITO (indium tin oxide) glass substrate. The system also includes components for switching and grounding the charge, allowing the structure to be reversibly actuated. In some embodiments, the system may optionally include sensors, such as optical or force sensors, which are configured to detect the deployment or conversion of the structure from its first state to its second state.

Embodiments here described encompass methods and systems for mechanically actuating an architected material of the present disclosure.

The method for actuating an architected material of the present disclosure comprises: providing the architected material of the present disclosure; applying a mechanical force to the plurality of discrete particle bodies; and causing the architected material to convert from a first state to a second state by inducing a strain that exceeds a programmable critical jamming strain. This conversion is reversible upon removal of the mechanical force, though the structure may exhibit hysteresis.

In embodiments herein described, the mechanical force is either a compressive force or a shear force or a combination of the two. The first state is a “fluid-like regime” characterized by a near-zero shear modulus and shear-thinning behavior. The second state is a “solid-like regime” characterized by a nonlinear stress-strain response and strain-stiffening behavior, which is induced when the kinematic degrees of freedom are exhausted at the critical jamming strain.

The system for actuating an architected material of the present disclosure is a mechanical actuator system, comprising: the architected material of the present disclosure; and a mechanical actuator communicatively coupled to the architected material. The mechanical actuator is configured to apply a mechanical force to the plurality of discrete particle bodies to cause the architected material to reversibly convert from a first state to a second state by inducing a strain that exceeds the programmable critical jamming strain.

In some embodiments, the mechanical actuator comprises one or more compression platens configured to apply a uniaxial compressive force. In other embodiments, the mechanical actuator comprises one or more gripping plates, such as solid gripping plates integrated into the material or custom-designed mounting plates, configured to apply a shear force. The actuator may be a universal testing machine, such as an Instron ElectroPuls E3000 or a displacement-controlled micromechanical testing system.

In further embodiments, the conversion states of the architected material can be programmed by combining multiple triggers. For example, a mechanical force can be applied to induce the Jamming Transition. While the structure is held in this jammed, solid-like state, an electrostatic charge (as described in the Third and Sixth Aspects) may then be applied. This combination of triggers may be used to lock the material in a specific jammed configuration, or to actively modulate the stiffness of the already-jammed structure. Additionally, while the structure is in a liquid-like state (no or little mechanical force applied), an electrostatic charge can be used to induce the jamming transition to transition the structure into a more solid-like state and, in some cases, cause the structure to change shape and/or orientation.

Accordingly, in embodiments, herein described the electrostatic charge and/or mechanical forces can be used to trigger other conversion states in a structure configured for such a response in accordance with the present disclosure

For example, in some embodiments, the first state can be a “fluid-like regime” characterized by a near-zero shear modulus, and the second state may be a “solid-like regime” characterized by a strain-stiffening response. In this embodiment, the application of the electrostatic charge may cause the particles to repel one another, exhaust their kinematic degrees of freedom, and “jam,” thereby converting the material from the fluid-like state to the solid-like state.

In another embodiment, for a structure configured for multi-stability, the first state may be a “collapsed” or flexible state, and the second state may be an “expanded” or stiff state. The application of the electrostatic charge may be used to trigger the conversion from the collapsed state to the expanded state, which may then be mechanically stable even after the charge is neutralized.

In some embodiments, the method comprises steps to fabricate and release the micro-scale structure; deposit a conductive coating, for example, sputtered metal; mount the structure on an insulating or ITO substrate with an electrical connection; apply a potential sufficient to charge the particles; observe the conversion from the first state to the second state; neutralize the charge to reverse the conversion; and optionally repeat the process cyclically.

In a preferred embodiment, this conversion state is an Actuated Shape-Morphing transition, wherein the structure converts from a first “compact” or uncharged state to a second “deployed” or charged state. This transition is demonstrated to be rapid, for example, occurring in less than 0.1 seconds.

The corresponding system for actuating a structure of the present disclosure is an electrostatic actuator system, which can comprise: one or more structures according to the present disclosure; and an electrical voltage source communicatively coupled to the conductive material of the plurality of unit particles, wherein the electrical voltage source is configured to apply an electrostatic charge to the unit particles, causing the structure to reversibly convert from a first state to a second state.

In some embodiments, the system comprises the structure; a conductive pathway; a voltage source, such as a Van de Graaff generator or an HV DC supply; components for switching and grounding the charge; conductive leads and fixtures; and a substrate, such as an ITO glass substrate. The system may optionally include sensors, such as optical or force sensors, to detect the conversion to the second state.

In some embodiments herein described, the conversion state of the material can be induced by one or more triggers selected from one or more mechanical forces. In particular in embodiments where at least one of the one or more triggers is Mechanical Force (such as compression, shear, or oscillation), the trigger requires any of the tested structural materials (e.g., acrylic polymer) and results in triggering the Jamming (Fluid-like->Solid-like) conversion state by exhausting DOFs, or triggering the Preconditioning (Initial->Steady-State) conversion state via cyclic loading. in embodiments where at least one of the one or more triggers is Electrostatic Field, this trigger requires the material to be at a Micro-scale (e.g., ≤1 mm), have a Conductive Coating (e.g., ≈300 nm Cu), and have a Low Density to allow forces to overcome gravity. This trigger results in the Actuated Morphing (Compact->Deployed) conversion state, which is reversible upon charge neutralization and rapid (e.g., transition time <0.1 s).

In specific embodiments, these variables can be combined to provide an actuation workflow for achieving a specific function. For example, to achieve High Compressive Flexibility (Shock Absorption), the selected Topology “Lever” includes a Catenation Number (CN) of approximately 6 with Tetrahedral or Octahedral Symmetry, with the Load Axis aligned with compression, as demonstrated in the T-6-ring tested example. The Geometric Ratio “Knobs” for this recipe include using a Higher CR (such as from a low d/D of approximately 0.14) to raise the critical compressive strain

$$( {ϵ}_{c}^{*} ) ,$$

while the SR is tuned to set the final stiffness. In another specific embodiment, to achieve High Shear Flexibility (Morphing), the selected Topology “Lever” includes a CN of approximately 4 with Planar Symmetry, with the Load Axis aligned with in-plane shear, as demonstrated in the J-4-ring tested example. The Geometric Ratio “Knobs” for this recipe include using a Higher CR (such as from a low d/D) to raise the critical shear strain

$$( {γ}_{s}^{*} )$$

and using a Lower SR to promote rearrangement. In yet another specific embodiment, to achieve Multi-stability (Stiff-to-Flexible), the selected Topology “Lever” includes a CN of approximately 4 with Planar Symmetry and a Mechanism that relies on rotating-unit kinematics, as demonstrated in the J-4-square (L↔S states) tested example. This is presented as a specific property of this example and not a general rule. In a further specific embodiment, to achieve High-DOF (Actuation), the selected Topology “Lever” includes a CN of 4-6 with any high-DOF topology, and importantly, the Scale must be Micro-scale (e.g., less than or equal to 1 mm side length), as demonstrated in the J-4-ring μ-PAM example. The Geometric Ratio “Knobs” for this recipe include using a High CR to allow room for deployment and a Low SR to reduce particle mass so electrostatic forces can dominate gravity.

In some embodiments, the structure may be configured for actuation by additional triggers beyond mechanical or electrostatic forces. These are expected to include a magnetic field, which is expected to require a constituent material property such as a ferromagnetic-doped polymer. Applying a magnetic field to such a structure is expected to cause particle alignment, stiffening, or controlled morphing.

Another trigger expected to be used in connection with the structures herein described is a thermal change, which is expected to require a constituent material property such as a shape memory polymer (SMP). A structure made from an SMP could be set in a temporary “compact” state and then, upon heating, be triggered to cause self-folding or a one-way deployment to its permanent “deployed” state.

Other additional triggers are expected to include environmental changes, requiring a solvent/humidity-responsive polymer or a light-responsive polymer to potentially cause swelling, folding, or deployment based on chemical or optical stimuli.

In some embodiments, to achieve a stable and repeatable mechanical response, the materials can undergo preconditioning or training methods. The documents describe this as applying cyclic loading, which causes the material to “stabilize into a steady-state response” with a smaller, consistent hysteresis loop after the first few cycles. This behavior is noted as being similar to the “Mullins effect” observed in rubbery materials, and it is also conceptually similar to the “training” required to set a stable transformation pathway in shape-memory alloys

In some embodiments, the materials also exhibit orientation-dependent anisotropy, meaning their properties are not the same in all directions due to their “crystalline network-based designs”. This is demonstrated by “gravity relaxation outlines,” where spherical samples of J-4-rings will settle into different final shapes depending on which crystallographic axis (<100>, <110>, or <111>) is oriented vertically. This anisotropy also affects “shear symmetry,” where rotating the sample relative to the loading direction results in a “pronounced anisotropic” shear response. This is a common property in many engineered and natural materials, such as wood, which is significantly stronger along its grain than across it.

Accordingly, the structure of the present disclosure can form an architected material, comprising: a plurality of discrete particle bodies, each particle body formed from one or more structural members. In the architected material of the present disclosure the plurality of particle bodies are mechanically interlocked to form a three-dimensional network with inter-layer linkages.

In the architected material herein described, the architected material is configured to exhibit a dual mechanical response, transitioning from a first state (such as a fluid-like regime characterized by a near-zero shear modulus) to a second state (such as solid-like regime characterized by a nonlinear stress-strain response), the transition occurring at a programmable critical jamming strain.

In some embodiments, 3D polycatenated architected materials (PAMs) are described.

PAMs are structures formed by 3D tessellation of interlocking particles. The particles can be 2D loop/ring structures (such as circles, ovals, squares, diamonds, triangles, etc.) or they can be 3D cage structures (3D structures formed by multiple loops).

In some embodiments, the PAMs have 3D strain redistribution, inter-layer cohesiveness, and/or tunable energy absorption.

In some embodiments, the PAMs have controllable kinematic degrees of freedoms (DOFs). Such units are symmetrically (along different axes) catenated with their neighbors that are free to move relative to each other within the bound of the interlocking mechanisms. Local variations in the particles' geometry affects the internal DOFs, which, in turn, control the global deformability and effective response of the bulk.

In some embodiments, the PAMs global mechanical behavior transitions from fluid-like to solid-like and from shear-thinning to shear-thickening, as a function of the applied loading. The unique behavior of PAMs arises from strong surface interactions between adjacent particles, for example, in contacts and relative sliding. These interactions can be further leveraged at smaller scales, by increasing the surface-to-volume ratio of the samples.

In some embodiments, the PAMs are resilient to cyclic loading and have tunable energy absorption, with scalable responses that persist at both the macro- and micro-scales.

Architected materials of the present disclosure can be provided by a method for manufacturing an architected material of the disclosure. The method comprises: forming a plurality of unit particles by an additive manufacturing process, each unit particle comprising a particle body formed from one or more structural members and defining one or more interlocking openings at one or more nodes; wherein the forming is performed such that an interlocking structural member of one unit particle passes through an interlocking opening of an adjacent unit particle at a node, thereby creating a mechanically interlocked three-dimensional network patterned after a crystalline network topology.

In embodiments herein described, the method comprises an additive manufacturing build that maintains the designed clearances between particles. This can include a support strategy to avoid unintended bonds or fusing at the interlocks. The method further comprises a post-processing step to free the interlocks, such as a solvent clean or plasma etch to remove support material.

In embodiments where the structure is configured for electrostatic actuation, the method further comprises a step of applying a conductive coating to the plurality of unit particles, for example, by sputtering approximately 300 nm of copper. After post-processing, the method may include an inspection for particle mobility to ensure the kinematic degrees of freedom are restored, as well as dimensional metrology of the d/D ratio and Clearance Ratio (CR), and mechanical couponing (such as compression, shear, or rheology tests) to verify the resulting mechanical behavior.

An example manufacturing process for these structures begins with a controller executing a digital build file on an additive manufacturing (AM) device, such as one using two-photon lithography, Multi Jet Fusion (MJF), Selective Laser Sintering (SLS), or Selective Laser Melting (SLM). The AM device applies the specified process parameters (e.g., laser power, scan speed, exposure paths) to fabricate the structure. Several essential practices are critical during this fabrication phase: the designed clearances between particles must be precisely maintained, and any unintended fusing at the interlocks must be prevented.

Following the AM build, a post-processing stage is required to remove support materials, which may involve applying a conductive coating (the “coat” step) and procedures like a solvent bath or plasma etch. This step continues until the mobile kinematic degrees of freedom (DOFs) of the particles are fully restored. Before the material is ready for use or testing the “measure/update” step), a final quality check is necessary to confirm the mobility at the nodes and verify the clearances. This can include dimensional metrology of the d/D ratio . . . and mechanical couponing (such as compression, shear, or rheology tests) to verify the resulting mechanical behavior and update the digital twin.

Additive manufacturing build with designed clearances; support strategy to avoid unintended bonds; post-processing (solvent clean, plasma etch) to free interlocks; inspection for particle mobility; dimensional metrology of d/D and CR; mechanical couponing (compression/shear/rheology).

Embodiments here described encompass methods and systems for actuating an architected material of the present disclosure.

The method for actuating an architected material of the present disclosure comprises: providing the architected material of the present disclosure, wherein the plurality of discrete particle bodies are coated with a conductive material; applying an electrostatic charge to the plurality of discrete particle bodies; and causing the architected material to reversibly convert from a first state to a second state due to electrostatic repulsion between the particle bodies. In a preferred embodiment, this conversion is a compact-to-deployed shape change. In some embodiments, the method comprises steps to fabricate and release the micro-scale structure; deposit a conductive coating, for example, sputtered metal; mount the structure on an insulating or ITO substrate with an electrical connection; apply a potential sufficient to charge the particles; observe the conversion; neutralize the charge to reverse the conversion; and optionally repeat the process cyclically. The method may further comprise controlling environmental factors, as humidity control can affect charging.

The system for actuating an architected material of the present disclosure is an electrostatic actuator system, comprising: the architected material of the present disclosure; and an electrical voltage source communicatively coupled to the conductive material of the plurality of unit particles, wherein the electrical voltage source is configured to apply an electrostatic charge to the unit particles, causing the structure to reversibly convert from a first state to a second state. In some embodiments, the system comprises the structure; a conductive pathway; a voltage source (e.g., Van de Graaff generator, HV DC supply); components for switching and grounding the charge; leads or fixtures; and a substrate (e.g., ITO glass). The system may optionally include sensors (e.g., optical, force) to detect deployment and components for optional charge management, such as resistors or bleed paths.

In some embodiments, a system can be configured for the mechanical characterization or actuation of the architected material is provided. The system can comprise one or more architected material structures, and a set of fixtures configured to interface between the material and a mechanical testing machine. These fixtures may include one or more gripping plates configured to be integrated with or attached to the architected material to apply a shear force, or specialized compression platens.

In further embodiments, the conversion states of the architected material may be programmed by combining multiple triggers. For example, a mechanical force (such as a compressive or shear load) may be applied to the structure to induce the Jamming Transition (fluid-like to solid-like state). While the structure is held in this jammed, solid-like state, an electrostatic charge may then be applied. This combination of triggers may be used to lock the material in a specific jammed configuration, or to actively modulate the stiffness of the already-jammed structure by using electrostatic repulsion to alter the inter-particle contact forces. Conversely, an electrostatic charge may be applied first to deploy the structure into a “deployed” state, which is then subsequently subjected to a mechanical load to induce jamming in that deployed configuration.

In embodiments herein described, the plurality of unit particles may be fabricated from any constituent material suitable for the chosen additive manufacturing process and end-use application. The properties of the architected material arise primarily from its topology and geometry rather than the constituent material alone. However, the material is selected to provide the desired structural integrity, stiffness, or functional response.

Exemplary constituent materials for forming the particle bodies include, but are not limited to, polymers, metals, ceramics, or composites. In specific embodiments, the material is a polymer, such as an acrylic polymer or a photopolymer resin. Exemplary photopolymer resins include those known commercially as Visijet M3, M2R-Clear, M2R-TN, and Nanoscribe IP-S. In other embodiments, the material may be Nylon, a Thermoplastic Polyurethane (TPU), or a metal. The selection of the material may be paired with the manufacturing process, such as using Nylon or TPU with Selective Laser Sintering (SLS), or a metal with Selective Laser Melting (SLM).

In embodiments configured for specific conversion states, the material can be selected or modified to respond to a trigger. For structures configured for electrostatic actuation, the plurality of unit particles are fabricated from or, more preferably, coated with a conductive material. In a specific embodiment, the unit particles are fabricated from a low-density polymer and are coated with a conductive material such as copper (Cu), for example, at a thickness of approximately 300 nm. In other optional embodiments, the constituent material may be selected to be a functional polymer. For example, to configure the structure for actuation by a magnetic field, the material may comprise a ferromagnetic-doped polymer. To configure the structure for actuation by a thermal trigger, the material can comprise a shape Memory Polymer (SMP). In other variations, the material may be a solvent-responsive polymer, a humidity-responsive polymer, or a light-responsive polymer to provide for other modes of actuation.

In embodiments where the constituent material is a ferromagnetic-doped polymer, this material is a composite comprising a non-magnetic polymer matrix and a filler of magnetic micro- or nanoparticles.

The polymer matrix can be a thermoplastic suitable for additive manufacturing processes such as Selective Laser Sintering (SLS) or Fused Deposition Modeling (FDM), and may be selected from materials including, but not limited to, Nylon (e.g., PA-11, PA-12), Thermoplastic Polyurethane (TPU), Polylactic Acid (PLA), or Acrylonitrile Butadiene Styrene (ABS). Alternatively, the matrix may be a photocurable resin suitable for processes like two-photon lithography or stereolithography, such as an acrylate or epoxy-based resin.

In embodiments, comprising a ferromagnetic-doped polymer, the magnetic filler is dispersed within this polymer matrix. The filler may consist of hard magnetic particles (which are permanently magnetized), such as Neodymium-Iron-Boron (NdFeB) or Samarium-Cobalt (SmCo), or soft magnetic particles (which become magnetized only in the presence of an external field), such as iron oxide (e.g., magnetite, Fe3O4), carbonyl iron powder (CIP), or other ferrites. The particles may have a size in the range of nanometers (nanoparticles) to tens of micrometers (microparticles). The loading concentration of these particles within the polymer matrix can vary, for example, from 5% to 70% by weight or volume, depending on the desired magnetic response and the rheological constraints of the manufacturing process.

For thermoplastic-based materials, the magnetic particles are typically compounded with the polymer pellets via twin-screw extrusion to create a homogenous feedstock filament (for FDM) or powder (for SLS). For photocurable resins, the magnetic particles are dispersed into the liquid resin using methods such as high-shear mixing or ultrasonication to create a stable suspension or ‘ferro-resin’ prior to fabrication.

When this composite material is formed into the polycatenated structure, it becomes responsive to an external magnetic field. When a field is applied, the magnetic particles experience a torque that attempts to align their magnetic dipoles with the external field lines. This collective alignment creates an internal stress within the polymer matrix, restricting the relative motion of the polymer chains and, on a larger scale, constraining the kinematic degrees of freedom (DOFs) of the unit particles. This results in a rapid and reversible increase in the material's bulk stiffness, effectively converting the structure from a flexible, fluid-like state to a solid-like, ‘jammed’ state. Furthermore, if hard magnetic particles are used, they can be pre-magnetized in a specific pattern during fabrication, enabling the structure to fold, twist, or morph in a pre-determined way upon application of a magnetic field.

In embodiments where the constituent material is a Shape Memory Polymer (SMP), the material is a (thermo) responsive polymer that exhibits a shape-memory effect. This effect is governed by the material's molecular architecture, which typically consists of two distinct components: (1) permanent, covalent or physical cross-links that define the permanent shap (the “memory”) and (2) temporary, switchable segments or ‘switching domains’ that define the temporary shape

The shape-memory cycle involves a thermo-mechanical programming process. First, the SMP structure is fabricated in its permanent, desired shape (e.g., the ‘deployed’ state). The structure is then heated above its characteristic transition temperature (Tₜᵣₐₙₛ), which is often the glass transition temperature (T_{g}) or melting temperature (Tₘ) of the switching domains. While held at this high temperature, the material becomes soft and is mechanically deformed into a new, temporary shape (e.g., a ‘compact’ or ‘collapsed’ state). The structure is then cooled below Tₜᵣₐₙₛ while the deformation is maintained. This cooling ‘freezes’ the switching domains, locking the structure into the temporary shape even after the external force is removed.

Actuation is achieved by subsequently reheating the structure above Tₜᵣₐₙₛ. This releases the frozen—in strain, and the elastic energy stored in the permanent cross-links provides the driving force for the material to autonomously recover its original, permanent shape. This enables functionalities such as self-folding or one-way deployment.

A variety of SMPs are compatible with additive manufacturing. For high-resolution processes such as two-photon lithography (2PP) or micro-stereolithography, suitable materials include photocurable acrylate-based or epoxy-based resins formulated to have a distinct T_{g} that serves as the Tₜᵣₐₙₛ. For macro-scale fabrication using processes like Fused Deposition Modeling (FDM) or Selective Laser Sintering (SLS), ** Thermoplastic Polyurethanes (TPUs) ** are a common choice, as their hard and soft segments can be engineered to create a well-defined thermal transition. Other suitable materials may include polycaprolactone (PCL) based systems or other thermosetting SMPs.

When this material is used to fabricate the polycatenated structure of the present disclosure, the as-fabricated, interlocked network constitutes the permanent shape. The programming process allows this entire cohesive structure to be collapsed into a dense, temporary state, which can later be actuated by heat to deploy, for example, a medical stent or an aerospace component, upon reaching its target destination.

In embodiments where the constituent material is a solvent- or humidity-responsive polymer, the material is typically a hydrogel. A hydrogel consists of a cross-linked network of hydrophilic polymer chains that can absorb large quantities of a solvent (such as water) without dissolving, resulting in significant volume expansion. For additive manufacturing, these materials are often fabricated from photocurable resins such as poly(ethylene glycol) diacrylate (PEGDA), poly(2-hydroxyethyl methacrylate) (PHEMA), or formulations containing acrylic acid or gelatin methacrylate (GelMA). These liquid resins, containing monomers, cross-linkers, and a photoinitiator, are selectively cured using processes like stereolithography or two-photon lithography (2PP) to form the solid, cross-linked hydrogel structure. When the fabricated polycatenated structure is exposed to water or high humidity, the structural members swell. This swelling increases the volume and thickness (e.g., the ‘d’) of the members, which in turn reduces or eliminates the clearances at the interlocking nodes. This exhaustion of the kinematic DOFs causes the structure to convert from its initial flexible, ‘un-jammed’ state to a rigid, ‘jammed’ state, effectively “locking” the structure.

In embodiments where the material is light-responsive, it typically incorporates photo-switchable molecules, or chromophores, such as azobenzene. These molecules undergo a reversible conformational change (trans-cis isomerization) upon illumination. Upon exposure to light of a first wavelength (e.g., UV light, ~365 nm), the molecule switches from its linear trans state to a bent cis state, inducing a macroscopic contraction in the polymer. This process is reversible by illumination with a second wavelength (e.g., visible light, >450 nm), which switches the molecule back to the trans state, causing the material to expand. To be additively manufactured, these azobenzene moieties are commonly incorporated into a Liquid Crystal Elastomer (LCE) network. This forms a photocurable ‘azo-LCE’ resin that can be printed via 2PP or high-resolution Digital Light Processing (DLP). During fabrication, the liquid crystal domains are aligned (e.g., via shear forces or an applied field) and then cross-linked into a solid. When this material is formed into the polycatenated structure, illumination with UV light will cause the structural members to contract, inducing a collective shape change (e.g., folding or collapsing) in the entire assembly. This converts the structure from an ‘expanded’ first state to a ‘collapsed’ second state, enabling remote and reversible optical control over the material's shape.

In further embodiments, the structure or architected material of the present disclosure is incorporated as a component into a larger system or device.

In one such embodiment, an impact protection system is provided. The system comprises a component configured to absorb energy, wherein the component comprises a structure according to embodiments herein or an architected material according to embodiments herein. The high compressive flexibility and energy absorption of specific topologies, such as the T-6-ring, make the material ideal for this purpose. The impact protection system may be, for example, a protective case for electronics, a shock absorber, a vehicle crash structure for a car or aircraft, a blast protection system for military applications, a seismic damper for buildings, or personal protective equipment such as a helmet or body armor.

In another embodiment, a soft robotic system is provided. The system comprises at least one reconfigurable component, wherein the component comprises a structure according to any one of the embodiments herein described or an architected material according to any one of the embodiments herein described. The high shear flexibility of topologies such as the J-4-ring, or the actuation of micro-scale or prophetically-triggered embodiments, allows the component to function as a flexible joint, a morphing surface, or an actuator.

In another embodiment, a morphing architecture is provided. The system comprises at least one reconfigurable component, wherein the component comprises a structure according to any one of the embodiments herein described or an architected material according to any one of the embodiments herein described. This may be used in, for example, aerospace applications for vibration attenuation in optical systems or for deployable structures.

In another embodiment, a flexible medical implant is provided. The implant comprises a structure according to any one of the embodiments herein described or an architected material according to any one of the embodiments herein described. The tunable flexibility and biocompatibility of certain constituent materials (e.g., TPU, PCL) make it suitable for use as a flexible graft or a deployable stent (as described in Example 19).

The systems provided herein for realizing or employing the architected materials described in the present disclosure may be supplied in the form of a kit of parts. In some embodiments, the kit comprises one or more pre-fabricated structural units-such as interlocking rings, cages, or other particle elements-adapted to be assembled or tessellated into a polycatenated network having the desired topology and mechanical response. The kit may further include auxiliary components such as substrates, conductive coatings, binders, or anchoring platforms for assembling or actuating the architected structures, together with optional instructions or specifications regarding lattice configuration, unit orientation, or particle geometry. In certain embodiments, the kit also comprises electronic, electrostatic, or mechanical drivers suitable for inducing reversible deformation, jamming, or shape-morphing of the assembled system. Each part of the kit can be stored or provided separately to facilitate transport, integration, or substitution of selected components, and may be configured for manual or automated assembly into a three-dimensional polycatenated architected material at the macro- or microscale.3D polycatenated architecture materials and related compositions, methods and systems, herein described herein described are further illustrated in the following examples, which are provided by way of illustration and are not intended to be limiting.

### Examples

The 3D polycatenated architecture materials and related compositions, methods and systems, herein described, are further illustrated in the following examples, which are provided by way of illustration and are not intended to be limiting.

In particular, 3D polycatenated architecture materials and related compositions, methods and systems, herein described, according to exemplary methods and systems of the disclosure.

A person skilled in the art will appreciate the applicability and the necessary modifications to adapt the features described in detail in the present section, to additional 3D polycatenated architecture materials and related compositions, methods and systems, according to embodiments of the present disclosure. A skilled person will understand how to adapt the specific, materials, and methods used in the following examples to additional materials, and methods identifiable in view of the instant disclosure such as additional, plant cells, biomass, mediums, compression mold, fillers, culturing, compression and/or detection processes in accordance with the present disclosure.

The exemplary 3D polycatenated architecture materials and related compositions, methods and systems, herein described, exemplified in this section were prepared and analyzed using the following materials and methods.

Materials and Fabrication of Macroscopic PAMS In this study, eight representative PAMs were fabricated: four composed of 2D particles (FIG. 10, panels E-H) and four composed of 3D particles (FIG. 10, panels M-P), using a brittle acrylic polymer using additive manufacturing. Specifically, PAM samples were fabricated from 3D Systems employing the Multi Jet Fusion fabrication process, sourced from a commercial 3D printing service (Shapeways). Materials used in this study are Visijet M3, M2R-Clear, and M2R-TN. All mechanical tests were conducted using PAMs fabricated using Visijet M3. Visijet M3 has a density of 1.02 g/cm³, a tensile modulus of 1463 MPa, and a tensile strength of 42.4 MPa [49]. Upon receiving the samples, some had remaining supporting wax materials, which could (i) alter the particle geometries and (ii) limit particles' DOFs. These wax materials can be selectively dissolved by soaking the samples in isopropanol solution for 2 min at a temperature between 4° and 60° C. All PAM samples were manually checked to ensure no remaining particle agglutination before mechanical characterizations. To test PAM samples under shear loading (in simple shear and rheology tests), two solid gripping plates can be integrated in the sample's design and fabrication (FIG. 11). The plates partially overlap with the particles at the top and bottom surfaces and were used to mount the samples to the loading grips and distribute the load uniformly across all particles on the surfaces. Notably, PAMs can also be fabricated using different materials such as Nylon (selective laser sintering, SLS), TPU (SLS), or metal (selective laser melting, SLM) (FIG. 25). Each sample was designed to be a cube with side ca. 50 mm (FIG. 10, panels E-H, M-P). To maintain 0.1 mm particle-particle separation, wax can be used as support materials and later dissolved.

Structural Characteristics of PAMs PAMs are characterized by their catenation number (CN), defined as the number of neighboring particles that are catenated with the center one, represented using ball-and-stick models (FIG. 10, panels A-D, I-L) [50]. The same CN can represent multiple local particle arrangements. For example, CN=4 in a planar arrangement maps to a network with a nbo topology, while CN=4 in a tetrahedral (Td) arrangement maps to a network with a dia topology [36].

J-4-ring (FIG. S4A) and J-4-square (FIG. 10, panel B) were designed to compare the influence of particle geometry while keeping the PAM topology constant. T-6-ring (FIG. 10, panel C) and T-6-HEX (FIG. 10, panel D) served as another pair of comparison. J-4-OCT (FIG. 10, panel I) and S-6/2-OCT (FIG. 10, panel J) were designed to compare the influence of PAM topology while keeping the particle geometry constant. D-4-TET (FIG. 10, panel K) and C-6-TT (FIG. 10, panel L) were designed because they are two results of geometric transformations from T-6 topology D-4-TET emerge when every two tetrahedral clusters catenated through 3-fold axes are transformed into a pair of tetrahedra; while C-6-TT emerge when every two tetrahedral clusters catenated through 2-fold axes are transformed into a pair of truncated tetrahedra (FIGS. 8A-8B). Notably, J-4-square has two stable configurations: an expanded, stiff configuration (L); and a collapsed, flexible configuration(S). Configuration L can transition into S via an auxetic mechanism like the ‘rotating squares behavior’ [51], which can be trained by cyclic application of external loading. To minimize structural randomness, standardize the maximum permissible particle thicknesses to maintain clearances as 0.1 mm in all designs except for J-4-OCT and S-6/2-OCT.

Quasi-static Compression and Shear Tests Quasi-static uniaxial compression and simple shear experiments were conducted using an Instron ElectroPuls E3000 universal testing machine. This testing machine was equipped with an ATI Mini85 multi-axial Force/Torque sensor capable of measuring both force and torque along three mutually perpendicular axes. The load cell's capacity was 1.9 kN and 3.8 kN for force measurements in the X, Y, and Z directions respectively, and 80 Nm for torque measurement along all three axes. For the quasi-static compression trials, cubic specimens were carefully positioned between the compression platens. The orientation ensured that the centerline of the specimen was aligned with the centerline of the load cell. To mount the shear test specimens in the Instron machine, a pair of custom-designed mounting plates was affixed to the solid gripping plates (3D-printed alongside the specimen). The specimens were oriented such that the solid gripping plates aligned with the sides of the specimen. Each mounting plate was subsequently connected to the corresponding upper and lower parts of the tensile grip in the Instron. This modification transformed the uniaxial tensile/compression test apparatus into a configuration suitable for simple shear testing.

The force-displacement responses of all specimens were recorded during quasi-static compression tests, at a displacement-controlled loading rate of 0.1/s, to maintain experimental consistency. In the compression tests, a series of multiple compression cycles were performed on a single specimen, each with a varying maximum loading strain spanning from 10% to 50%. Due to the inherently stochastic nature of PAMs, their dimensions exhibited variability in each instance, influenced by random internal arrangement patterns. Thus, dimensions of relaxed PAMs under gravity were measured multiple times, across various regions. The resultant average measurements were then utilized to compute effective stress and strain, accounting for this variability.

Numerical simulation of uniaxial compression experiments (LS-DEM) Numerical uniaxial compression experiments were conducted using LS-DEM (Level Set-Discrete Element Method). The model parameters, as well as the construction of digital twins, follows those reported in a previous work where LS-DEM was utilized to study the tunable mechanics of structured fabrics. The inter-particle friction between the ring particles was set as 0.25, same as in the previous work [30]. The inter-particle friction between the two platens and the ring particles was set to a small value of 0.1 [41], considering the smoothness of the loading frame in the experiments. The T-6-ring sample was first settled under gravity to equilibrium and then compressed by moving the top platen downward. The imposed displacement of the top platen, as well as the contact force exerted by particles to the top platen was recorded at each time step, and these two quantities were used to plot the stress-strain result. Note that the dimensions (height, width, and length) of the sample were calculated at the end of the gravity-settling step and then used to convert displacement into strain and contact force into stress.

The apparent compressive modulus (E*) of the sample at each strain value was calculated by fitting a polynomial to the stress-strain curve. For all simulated cases, we consider data starting from 4% strain and onward, excluding the initial stage (<4\% strain) where the stress is close to zero. The observation that E* scales as a power law with Z-Z₀ was found to be insensitive to the degree of fitting polynomial used (tested up to degree of five), and the scaling exponent was found to stay around 0.9 (see FIG. S10). Z₀=5 is picked approximately as the mean contact number of the “tapped” sample before undergoing uniaxial compression. (A “tapped” sample is one further subjected to a small-strain (5% in this work) loading and unloading cycle after settles to equilibrium under gravity, while a “as is” sample is one subjected to only settlement under gravity.) With Z₀=5 fixed, it is also found that the scaling exponent value is insensitive to the different order of the polynomial used here for fitting the stress-strain curve (and for subsequently calculating E*, see FIG. 16). Hence, one may regard the “tapped” configuration as being representative of the “marginally jammed” configuration, in terms of finding Z₀. Changing Z₀ to 5 (obtained from the initial configuration of the “tapped” case) from 2 (obtained from the initial configuration of the “as is” case) was able to collapse data from the “as is” cases onto the data of “tapped cases”. This observation suggests that the initial configuration of “tapped” may be used to determine the minimal contact number for the considered sample arrangement to gain structural rigidity.

FIG. 17 shows two additional particle-scale analysis of the “as is” 10% strain simulation case. The mean “engaged” friction between ring particles was computed as a function of cumulative strain (FIG. 17, panel a). The Gini coefficient of the magnitude of contact forces was also computed as a function of cumulative strain (FIG. 17, panel b), following the formula presented in “Quantifying interparticle forces and heterogeneity in 3D granular materials” [52]. The Gini coefficient has a value between 1 (totally heterogeneous) and 0 (totally homogeneous) and it is used here to quantify the heterogeneity of contact forces. It was observed that, first, during the loading phase, the engaged friction decreases, and the contact forces become less heterogeneous (the Gini coefficient decreases); second, after entering the unloading phase, the engaged friction increases, and the contact forces become more heterogeneous. Both observations agree with an experimental study on the contact force distribution evolution within a micro glass sphere assembly under uniaxial compression.

Rheology Tests The dynamic oscillatory rheological tests were performed using a torque-controlled Discovery HR20 Hybrid Rheometer, from TA Instruments which has axial force and torque limits of 50 N and 0.2 Nm with sensitivities at 0.005 N and 10-10 Nm, respectively. The experiment was carried out using a 40 mm diameter parallel plate tool, maintaining a constant temperature of 25° C. and a frequency of 0.5 Hz, with the torsional strain ranging from 10-6% to 100%. Cylindrical specimens of original height and diameter of 40 mm were employed. These specimens were designed with thin solid components at both ends, strategically incorporated to ensure steadfast attachment to the rheometer and prevent any slippage. To achieve this secure attachment, thin, robust double-sided adhesive tapes were utilized. Torsional rotation was applied to the specimen through the upper parallel plate, while the bottom side remained securely fixed. Data acquisition was facilitated using the TRIOS software. Data points lying within the strain range of 10-6% to 10-3%, were subjected to noise interference and were deliberately excluded from the study.

Numerical Simulation of rheological experiments (LS-DEM) Numerical frequency sweep experiments were conducted using LS-DEM. The model parameters, as well as the construction of digital twins, follows those reported in a previous work where LS-DEM was utilized to study the tunable mechanics of structured fabrics. The inter-particle friction for the J-4-ring was set as 0.25, same as in the previous work [30]. The inter-particle friction for the T-6-ring sample was increased to 0.5, to partially account for the noticeable surface roughness of the 3D-printed ring particles.

Each numerical experiment consists of two steps: settling under gravity until equilibrium and rotating under a given angular frequency. Note that, rings in the bottom layer were fixed during both steps, while those in the top layer were fixed during the first step and were rotated with respect to the sample's vertical central axis during the second step. The rotation in the second step was imposed according to an applied sinusoidal shear strain γ(t)=Asin (ωt), where A is the amplitude (set at 0.1 for all simulations) and w is the angular frequency. By doing so, we resemble as close as possible the experimental loading conditions. In practice, the rotation angle θ(t) and the speed \dot{θ}(t) were calculated, from which the spatial position, orientation, as well as translational and angular velocity of each ring in the top layer (which revolves around the rotation axis) can be determined. θ(t) and θ(t) were calculated according to the following two expressions:

$$θ ( t ) = \frac{γ H}{R} sin ( ω t ) ,$$

$$\dot{θ} ( t ) = \frac{γ H}{R} A ω cos ( ω t ) .$$

Above, H is the height and R is the radius of the cylindrical sample, determined from the sample's settled configuration. As rings in the top layer were moved via the imposed motion described above, the torque they felted, T(t), was computed. This is done by summing the cross products between the positions of them (with respect to the rotation axis) and the contact forces (projected onto the plane perpendicular to the rotation axis) felted by them. The shear stress T(t) was then computed according to the following expression:

$$τ ( t ) = \frac{T ( t ) R}{J} ; where$$

$$J = \frac{π {R}^{4}}{2}$$

is the polar moment of inertia of the sample (treated as a rigid cylinder). The maximum value τₘₐₓ was then determined from τ(t) and was used to find the complex viscosity η* based on the following expression:

$${η}^{*} = \frac{{τ}_{max}}{A ω}$$

For each simulation with a given angular frequency, is it possible to obtain get multiple values of τₘₐₓ (see FIG. 18 for a representative example), thereby calculating multiple values for η*. These values were averaged, and the mean values are recorded. Variations of η* due to variations in τₘₐₓ were found to be small (one order of magnitude smaller) compared to the mean value of η* and therefore do not need to be taken into account. For this work, the number of τₘₐₓ value varied from two to ten depends on the imposed angular frequency and out of consideration of computational costs. In general, the larger the angular frequency, the cheaper (in terms of computational cost) the simulation becomes for getting more values for τₘₐₓ.

At each location where τₘₐₓ is computed, also compute the mean contact number per particle [symbol] Z [symbol] and the mean particle velocity fluctuation δv by averaging all particles in a sample. The former is easy to get directly from LS-DEM simulation outputs. For the latter, take the particle velocity output and perform a spatial average to get δv. Here, consider δv along the azimuth direction only, and partition particles into different layers vertically (Zi, with z being the height coordinate location) and different annular bins (rⱼ, with r being the radial coordinate location) within each layer. Then, δv is computed as:

$$δ v = \frac{1}{{N}_{z}} {\sum }_{i = 1}^{{N}_{z}} \frac{1}{{N}_{r}} {\sum }_{j = 1}^{{N}_{r}} {〈 δ v 〉}_{{z}_{i} {r}_{j}}$$

where N_{z} and Nᵣ is the number of layers and bins per layer, respectively. [symbol] V [symbol] _{z}_{ᵢ}ᵣ_{ⱼ} the particle-averaged velocity fluctuation associated with a particular bin in a given layer, and it can be computed as the root square of the granular temperature [symbol] T [symbol] _{z}_{ᵢ}ᵣ_{ⱼ}

$${〈 δ v 〉}_{{z}_{i} {r}_{j}} = \sqrt{{〈 T 〉}_{{z}_{i} {r}_{j}}} , with$$

$${〈 T 〉}_{{z}_{i} {r}_{j}} = \frac{1}{{N}_{p , {z}_{i} {r}_{j}}} {{Σ}_{k = 1}^{{N}_{p , {z}_{i} {r}_{j}}} ( {v}_{k , r} - 〈 {v}_{k , r} 〉 )}^{2} .$$

Above, N_{p,z}_{ᵢ}ᵣ_{ⱼ} is the number of particles residing in that considered bin. Within this bin, V_{k,r} is the projected velocity along the azimuth direction of the k-th particle:

$${v}_{k , r} = \vec{{v}_{k}} \cdot \vec{{t}_{k}}$$

where \vec{vₖ} is the velocity vector of the k-th particle and \vec{tₖ} is the tangential unit vector along the azimuth direction associated with that particle. Lastly, [symbol] v_{k, r} [symbol] is the mean projected velocity, computed as the following:

$$〈 {v}_{k , r} 〉 = \frac{1}{{N}_{p}} {\sum }_{l = 1}^{{N}_{p}} {v}_{k , r}^{( l )}$$

where one can drop the z; and r; subscripts for simplicity.

Numerical Simulation of Critical Jamming Experiments The critical jamming simulations were conducted using the LS-DEM framework. Model parameter settings, including particle representation and inter-particle interactions, following the rheological simulations previously presented. In the shearing tests, the rings in the bottom layer were fixed, while a constant horizontal force was applied to the top layer in both leftward and rightward directions until the shearing strain reached a steady state. Shearing strains were recorded for both directions, and their mean value was taken. For the compressive straining tests, the rings in the left layer were fixed, and a constant horizontal force was applied to the right layer. Initially, a rightward force was applied to stretch the model to its maximum length, followed by a leftward force to compress the model to its minimum length. The average strain, calculated from both the stretched and compressed states, was reported (FIG. 19). Each simulation for a given configuration and loading condition was repeated multiple times, with the average of the resulting strains presented in FIG. 20.

Fabrication and Characterization of Microscopic PAMS The μ-PAM samples were fabricated by two-photon lithography using a commercial 3D printer (Nanoscribe GT2) and a commercial resin (Nanoscribe IP-S). The smaller samples used in micro-mechanical compression experiments were printed as designed, without adding additional mechanical supports. The larger samples used in electrostatic deployment experiments were printed by stitching multiple segments sequentially due to the limited field of view of the 25× objective used for two-photon lithography. To prevent drifting issues of the interlocked but unsupported structures due to the prolonged printing time for larger μ-PAM structures, we incorporated additional support structures (body-centered cubic lattices with lateral beam thickness of 500 nm) that overlapped with the μ-PAM lattices in the CAD design. The combined geometry was printed simultaneously so that the body-centered cubic scaffold provided additional support and confinement of the PAM during the printing process. The as-printed samples were immersed in propylene glycol methyl ether acetate (Sigma-Aldrich) for 1 hr and then in isopropanol for Ihr to rinse off the remaining uncured liquid resin. The cleaned samples were dried in air. The larger samples used in electrostatic deployment experiments were etched in a PIE Scientific Tergeo Plus Plasma Cleaner for 10-20 h (direct mode, oxygen flow rate 10 sccm, Ar flow rate 30 sccm, RF power 30 W, pulse duty cycle 50/255), to remove the supporting scaffold. Special attention was paid to increase uniformity of etching for all polymer surfaces so that the 500 nm thick body-centered cubic supporting lattices could be removed before substantially damaging the μ-PAM structure. A Keyence VHX-7000 Digital Microscope was used to inspect the etched μ-PAM samples every hour to complete removal of the supporting lattice and release of the interlocked particles in the polycatenated architecture.

For electrostatic deployment experiments, the fully released μ-PAM samples attached to ITO-coated glass substrates were coated with nominally 300 nm thick of Cu (measured on a planar substrate) using a Kurt Lesker PRO Line PVD 75 thin film sputtering system at 100 W DC power. The Cu-coated samples were attached to a Lethan Corporation Van De Graaff Generator using double-sided conductive Cu tapes. Digital photos and videos were taken when the Van De Graaff generator was being turned on and off.

For microscale mechanical testing experiments, the μ-PAMs were fabricated on 5 mm-by-5 mm Si substrates directly without the additional supporting scaffold. The uniaxial compression tests were conducted using a displacement-controlled micromechanical testing system (Kamrath Weiss Tensile & Compression Module). The device was equipped with custom compression grips, including an alumina flat punch with a tip of 2 mm in diameter, a load cell of 10 N, and a 3D-printed fixture to facilitate accurate and convenient mounting of the 5 mm Si substrates onto the compression module. Samples were tested at a strain rate of 0.5%/s. The experiments were performed under a Keyence VHX-7000 digital microscope for in situ video recording.

### Example 1: Conversion of Continuous Graph Topologies into PAMs

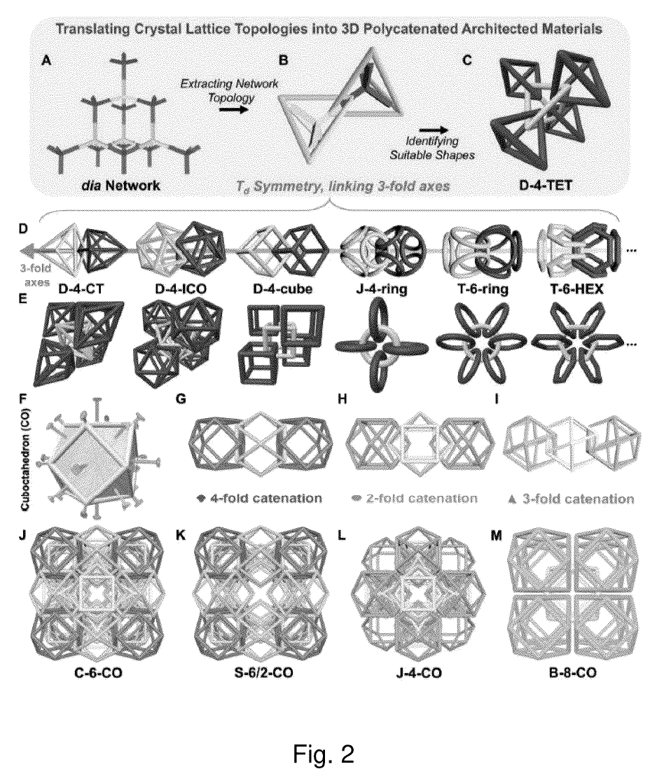

Traditional lattice structures can be mapped into continuous topological networks that consist of nodes and connections (FIG. 2, panel A). Starting from a chosen crystalline network, create periodically entangled toroidal, polygonal, or polyhedral caged particles (hereinafter referred to as ‘particles’) that can be tessellated into PAMs. The process begins by identifying node symmetries in the continuous networks and aligning them with particles that possess these symmetries (FIG. 2, panel B). These particles interlink with adjacent ones, replicating the original network connections (FIG. 2, panels A-C). Thanks to the enormous database of crystallographic symmetries, topological networks from databases like Reticular Chemistry Structure Resource (RCSR) can be transformed into polycatenated analogs (FIG. 7). A single node can be represented using particles with various shapes, such as polyhedral wireframes, polygon clusters, or torus clusters (FIG. 2, panel D). Depending on the nature of the constituent units, the topologies of resulting polycatenated architectures exhibit substantial variations (FIG. 2, panel E).

### Example 2: Generating 3D PAMs from Prescribed Particle Geometries

A given particle shape (e.g., cuboctahedron, CO) can exhibit multiple symmetry axes (FIG. 2, panel F), leading to several potential catenation environments (FIG. 2, panels G-I). By utilizing these catenations singularly or in combination, variety of PAMs can be created (FIG. 2, panels J-M), each with its own global topology. Employing a tripartite naming scheme, X-n-abc, for easy identification: ‘X’ for the network topology (Table 1), ‘n’ for the number of concatenations per particle, and ‘abc’ for the particle shape, either in full (lowercase letters) or as an acronym (uppercase letters). As an example, the label D-4-TET (FIG. 2, panel C) denotes a polycatenated diamond network (D) constituted from tetrahedral (TET) wireframes that interlock corner-to-corner with 4 neighboring particles (4).

TABLE 1
Name of PAMs' network topologies and analogous
chemical examples.

| Network topology | Equivalent | Chemical |
|---|---|---|
| of PAMs | RCSR symbol | example |
| J | nbo-b | Niobium monoxide (56) |
| T | pbz (hxg) | Polybenzene (57) |
| S | pcu-b | Colloidal cocrystal (58) |
| D | dia | Diamond (59) |
| C | pcu | Polonium (60) |

### Example 3: Mechanical Characterization of PAMs

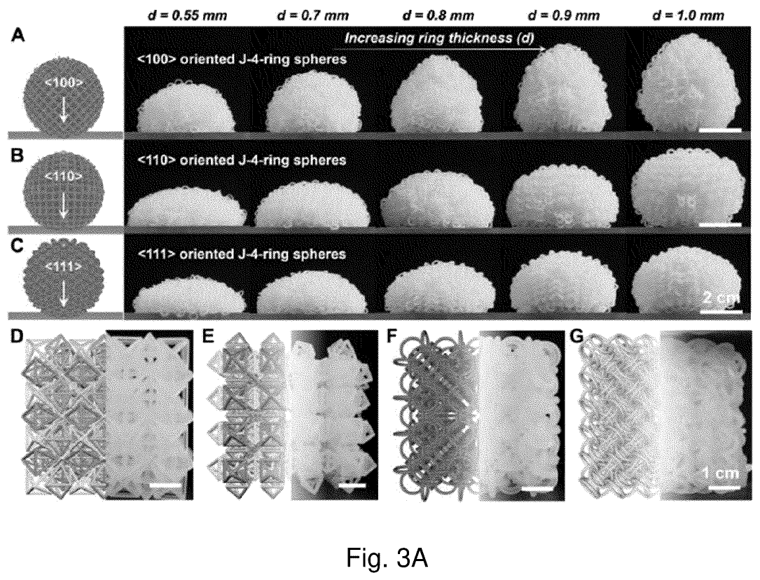

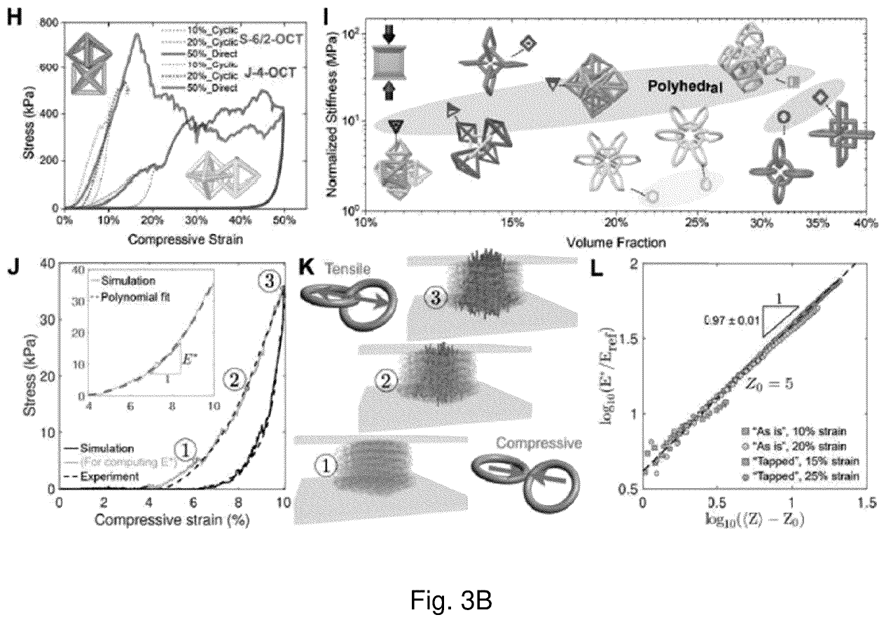

Given N×N×N arrays (where N, the array size, counts the number of unit cells per edge in the designed domain and consists of one or more interlocked particles, see Table 2) leading to different PAM geometries and topologies. The naming convention for these structures, as shown in Table 2, follows a<Topology code>-<CN>><Geometry>format (e.g., J-4-ring, T-6-HEX, D-4-TET, C-6-TT). For the J-4-square structure, two trained states are denoted: L (expanded/stiff) and S (collapsed/flexible). They can be fabricated by additive manufacturing with a brittle acrylic polymer leading to different PAM geometries and topologies. They can be fabricated by additive manufacturing with a brittle acrylic polymer. Selected are eight types of representative PAMs, four composed of 2D particles (FIG. 10, panel A-D) and four composed of 3D particles (FIG. 10, panels E-H). After fabrication and removal of support material, the PAMs relaxed under gravity and the originally ordered and periodic microstructure became irregular (FIG. 3A). Such gravity-induced shape deformations (hereinafter referred to as ‘relaxations’) of PAMs are highly dependent on their domain boundaries (e.g., spherical, FIG. 3A, panels A-C) and on the particle-to-particle clearance. For mechanical characterization, design cubic samples with sides ca. 50 mm (FIG. 3A, panels D-G; FIG. 10), and measure their designed and relaxed dimensions, as summarized in Table 2.

TABLE 2
Designed and relaxed dimensions of PAMs.

| | | | | | Designed | Relaxed |
|---|---|---|---|---|---|---|
| | Array | Measured | Measured | Measured | edge | volume |
| | size | width | depth | height | length | fraction |
| Name | (N × N × N) | (mm) | (mm) | (mm) | (mm) | (%) |
| J-4-ring | N = 5 | 54.50 | 54.50 | 54.00 | 55.00 | 31.84 |
| J-4- | N = 5 | 56.25/42.78 | 56.25/43.79 | 56.25/42.27 | 56.50 | 15.67/35.23 |
| square* | | | | | | |
| T-6-ring | N = 6 | 46.40 | 46.40 | 42.03 | 50.00 | 25.67 |
| T-6-HEX | N = 6 | 47.49 | 47.48 | 46.45 | 50.00 | 22.11 |
| J-4-OCT | N = 4 | 55.80 | 55.80 | 56.93 | 58.00 | 16.77 |
| S-6/2- | N = 4 | 54.20 | 54.20 | 52.40 | 56.00 | 10.87 |
| OCT | | | | | | |
| D-4-TET | N = 5 | 59.20 | 59.20 | 58.20 | 60.00 | 12.68 |
| C-6-TT | N = 6 | 47.96 | 47.96 | 47.96 | 49.00 | 32.74 |

To characterize their mechanical responses, conduct quasi-static uniaxial compression, simple shear, and rheology tests under different loading conditions. The mechanical response of PAMs emerges from a complex interplay of interactions across scales, ranging from (i) μm-scale inter-particle contacts, (ii) mm-scale particle deformation (e.g., bending, buckling and fracture), (iii) mesoscale layer-by-layer collapse, and (iv) cm-scale global deformations. Initially, all loadings induce the rearrangement of particles within the available kinematic DOFs and the redistribution of stresses within the volume, without damage to the particles. However, the particles' rearrangement leads to a bulk deformation of PAMs, which persists even after the removal of the external loads. As the particles reach a jammed state, further spatial reconfiguration becomes untenable. Beyond jamming, continued compressive forces result in particles' deformation, damage and fracture.

Under uniaxial compression (FIG. 3B, panel H; FIG. 12, panels A-F; FIG. 13), all samples exhibit a nonlinear stress-strain behavior with substantial loading/unloading hysteresis (energy absorption). This hysteric stress-strain relationship is likely influenced by three distinct mechanisms: (i) the rearrangement of catenated particles, (ii) the presence of friction at the contact, and (iii) at larger strains, particle deformation and damage. It is noted that, after relaxation, the particles' arrangement (i.e., their position and orientation) in PAMs is intrinsically disordered and varies from experiment to experiment. However, it is observed that PAMs present consistent values of effective stiffness, within a statistical distribution in the range of 10%.

A particularly interesting observation is the strain-stiffening response during the loading phase (FIG. 3B, panel J), which is reminiscent of the response of classical granular systems [38]. To understand and quantify the role of dynamic contact chains within PAMs, perform level-set discrete element method (LS-DEM) [39] [40]. While other potential computational methods exist, such as rigid multibody dynamics with nonsmooth contact/complementarity, contact-rich FEM for deformable particles (which is computationally heavy), Contact Dynamics (Moreau), hybrid DEM-FEM for particle deformation, or GPU-accelerated DEM codes, LS-DEM was used for its ability to handle arbitrary shapes and provide efficient contact handling. These simulations with particles of arbitrary shapes [30] [41]. First analyze the response of a T-6-ring sample, subjected to uniaxial compression [37]. Experimentally, under cyclic compression at lower strains, PAMs show a reduced peak stress and a hysteretic response with a progressively smaller area, then stabilize into a steady-state response after the first few cycles (FIG. 13).

Within the small to moderate strain regime (e.g., no particle fracture), numerical simulation quantitatively captures the steady-state stress-strain response (see FIG. 3B, panel J for an example). From the simulation result, further calculate the apparent compressive modulus (E*) at each loading step by fitting a polynomial to the stress-strain curve during the loading phase (FIG. 3B, panel J, inset). As the compression progresses, it is observed that the T-6-ring PAM “densifies” as more contacts (both tensile and compressive ones) form among particles (FIG. 3B, panel K) and carry larger contact forces (thicker and longer blue and red lines in FIG. 3B, panel K). To relate the macroscale strain-stiffening response with the particle-scale “densification” process, E* can be plotted as a function of [symbol] Z [symbol] -Z₀, where [symbol] Z [symbol] is the mean contact number (summing over cohesive and compressive ones) and Z₀ is the minimal contact number per particle for the sample to become structurally rigid [37] [42]. Z₀ depends on many factors such as particle friction, geometry, and catenation topology and is unknown for these catenated granular systems. Nevertheless, picking Z₀=5 can collapse data from different preparation protocols and loading strain ranges (FIG. 3B, panel L), which follows a power-law scaling as predicted by the critical phenomenon of jamming phase transition [42]. Here, the scaling exponent is very close to one, obtained by fitting a second-order polynomial to the stress-strain curve to get E *. This result, together with additional particle-scale analysis based on granular physics (see FIG. 17), suggests that the (small-strain) mechanics of PAMs share many features with that of classical granular materials. Note that, however, these LS-DEM simulation-based analyses will fail when particles deform or fracture.

### Example 4: Mechanical Tests

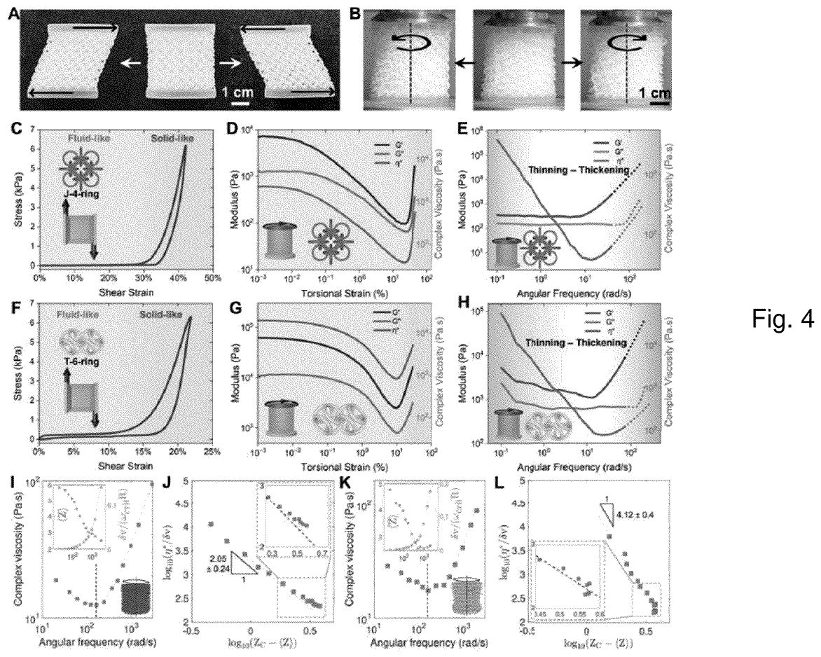

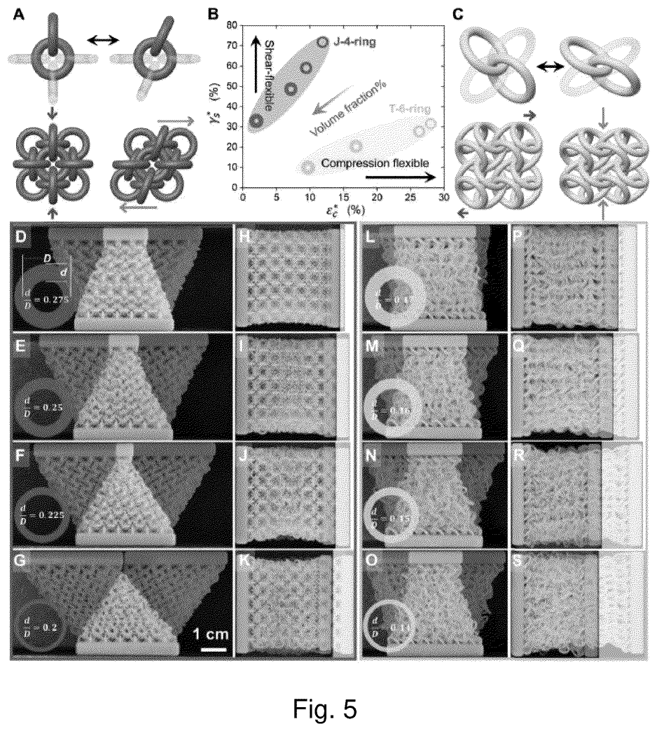

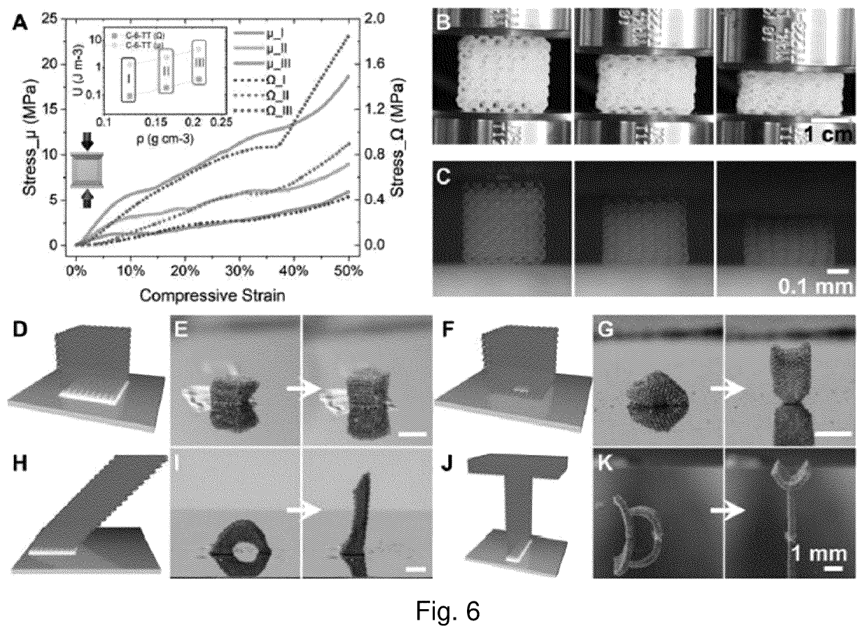

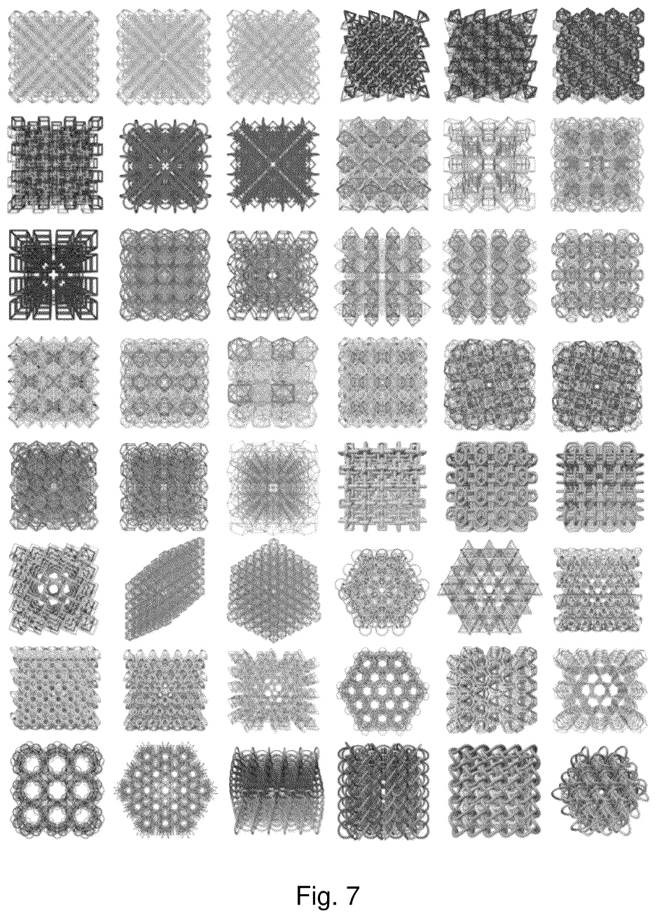

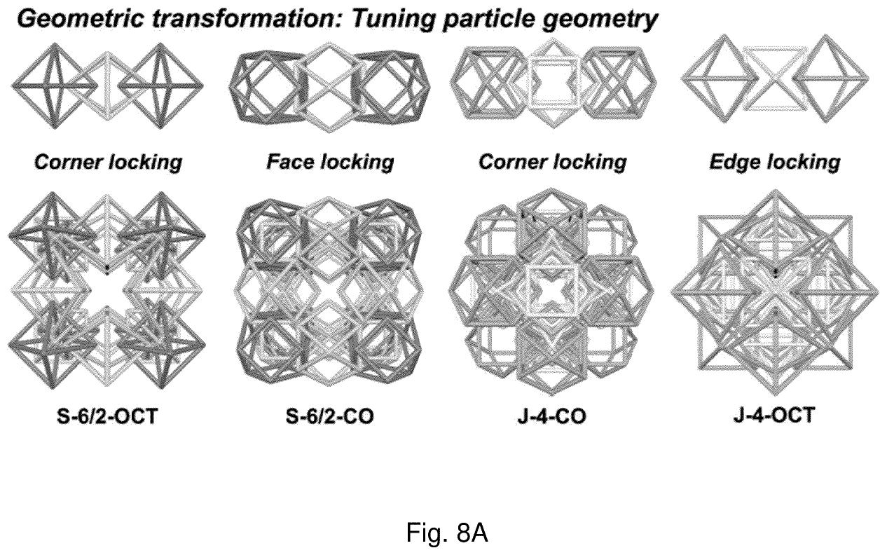

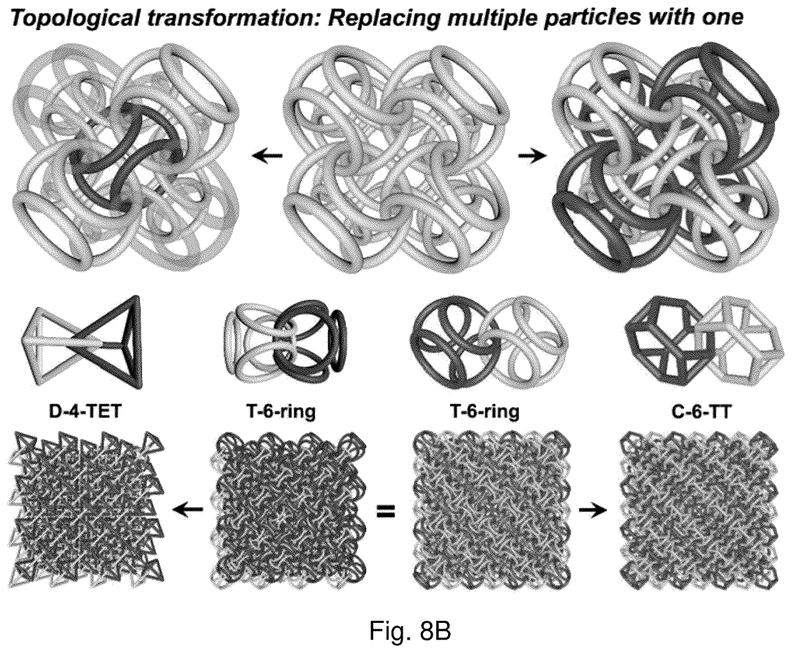

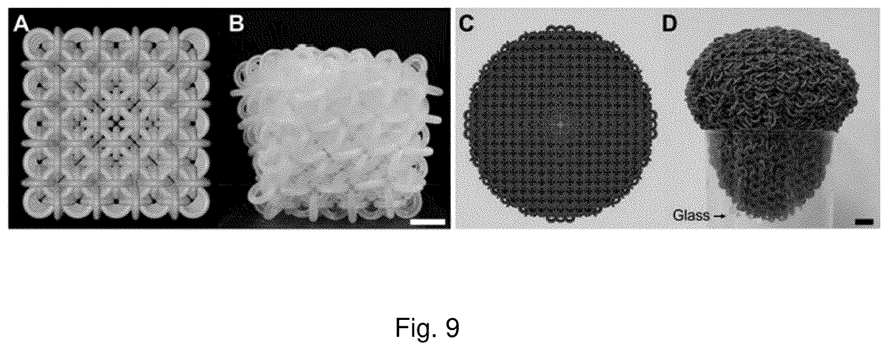

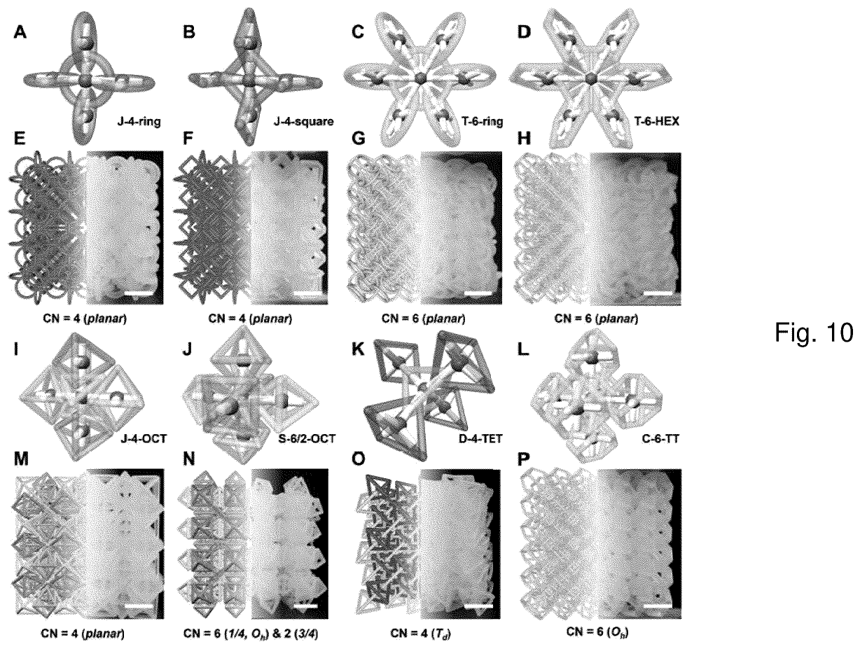

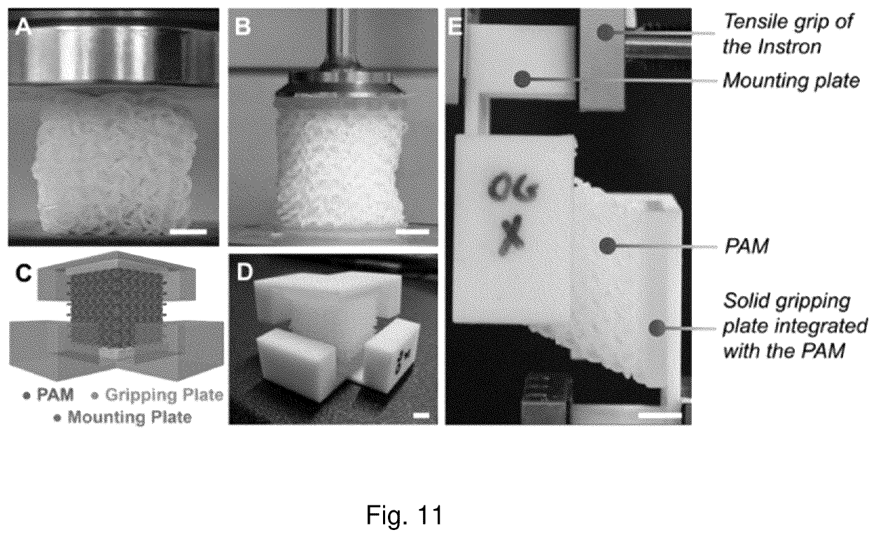

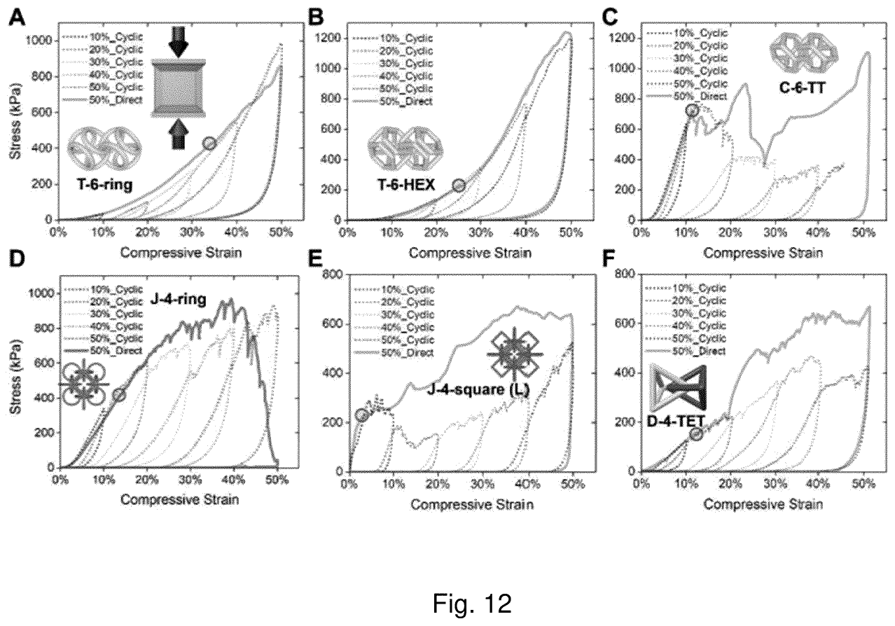

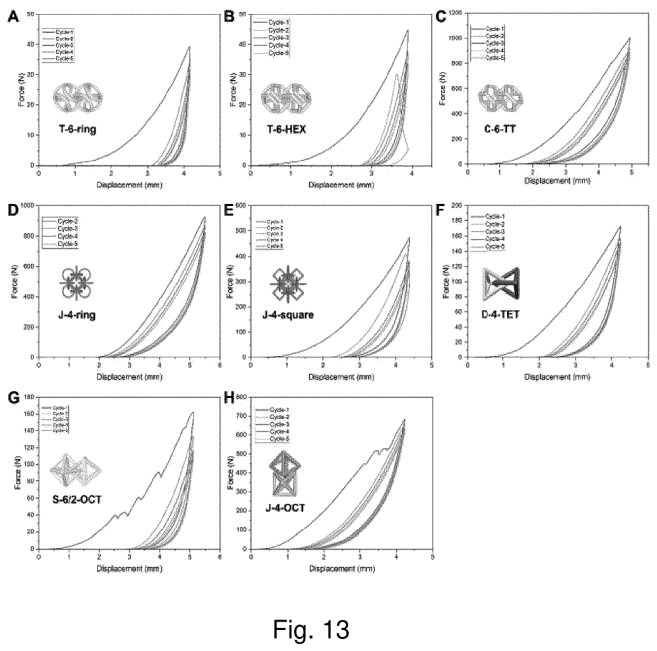

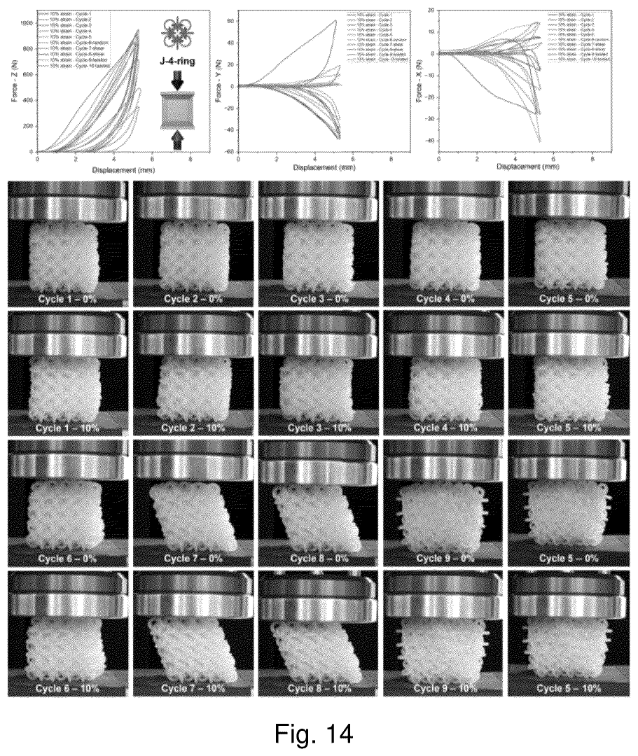

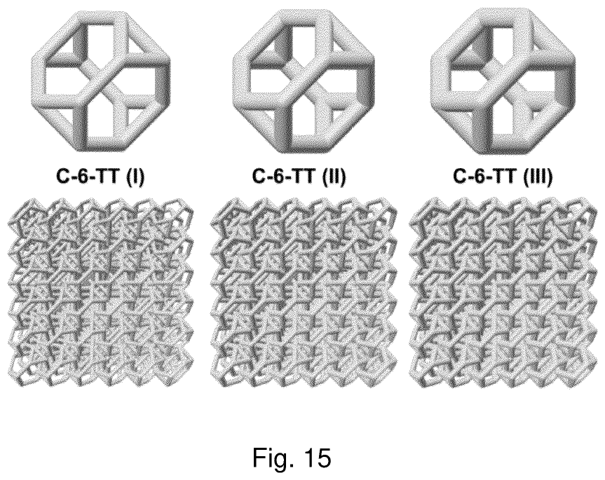

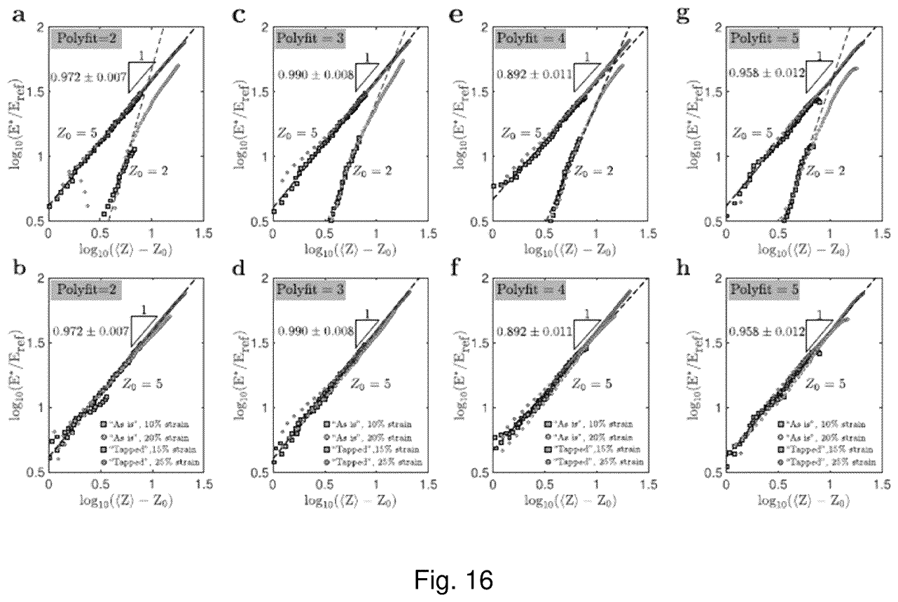

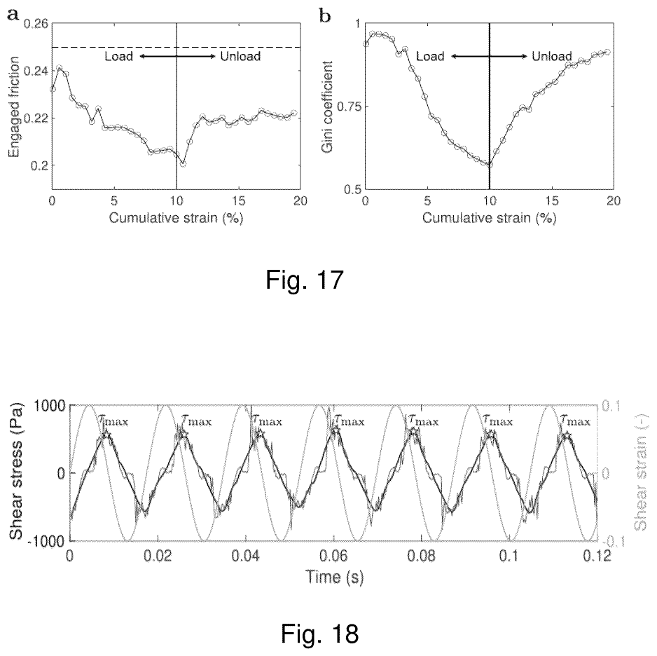

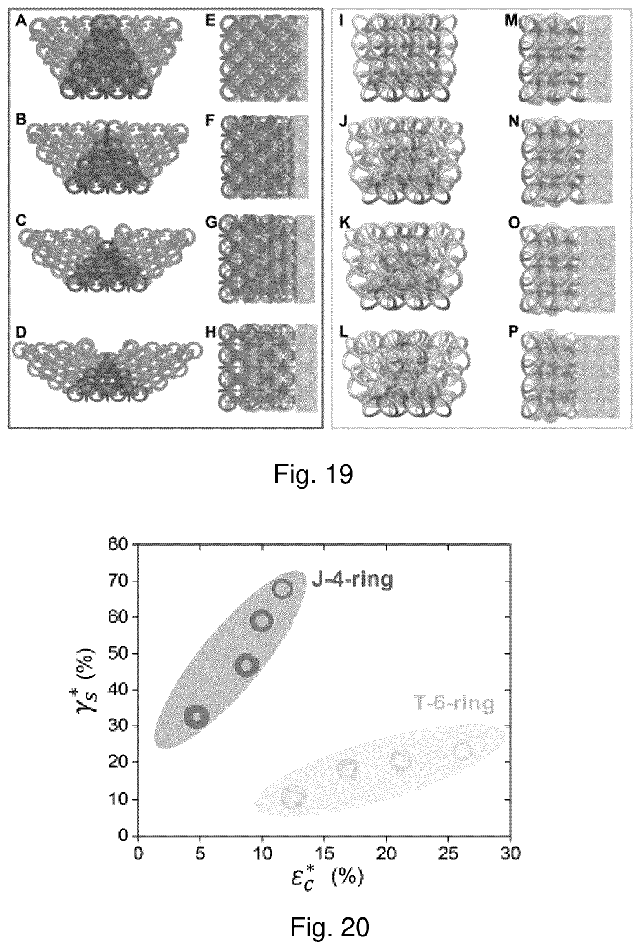

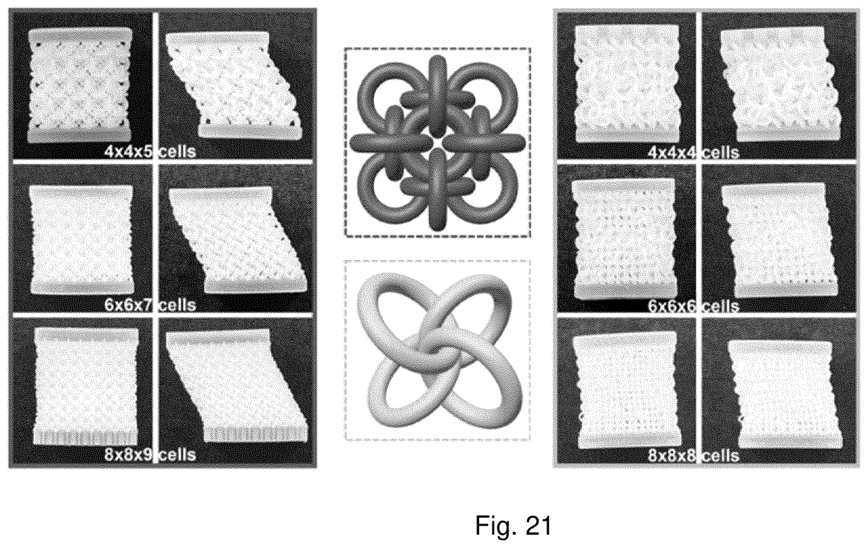

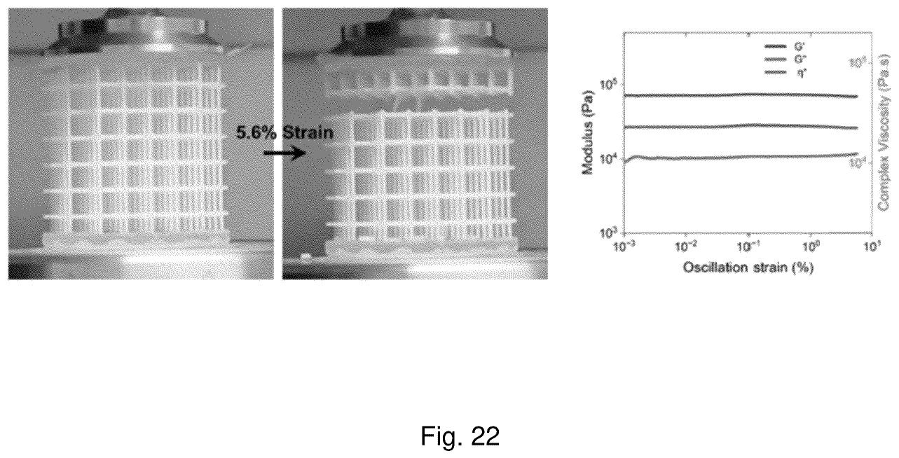

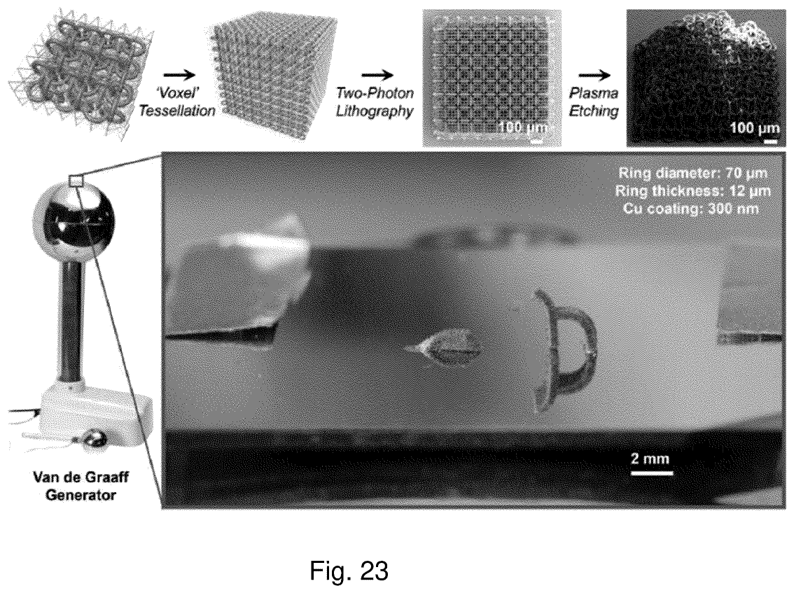

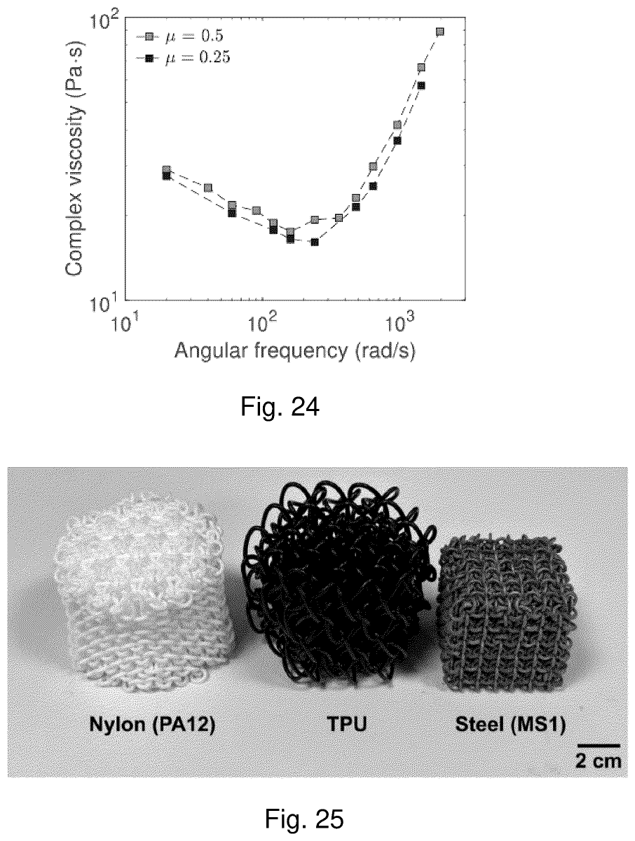

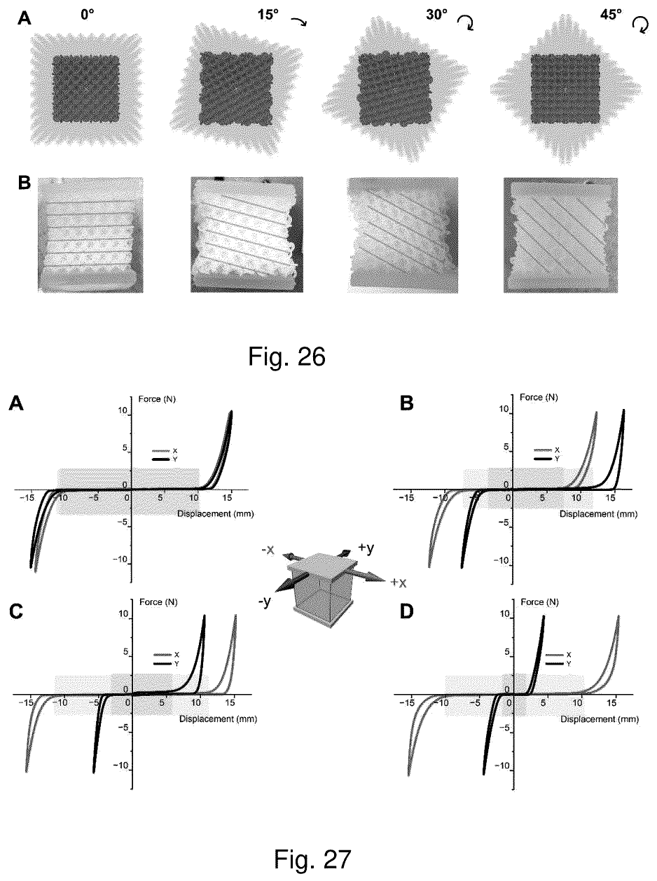

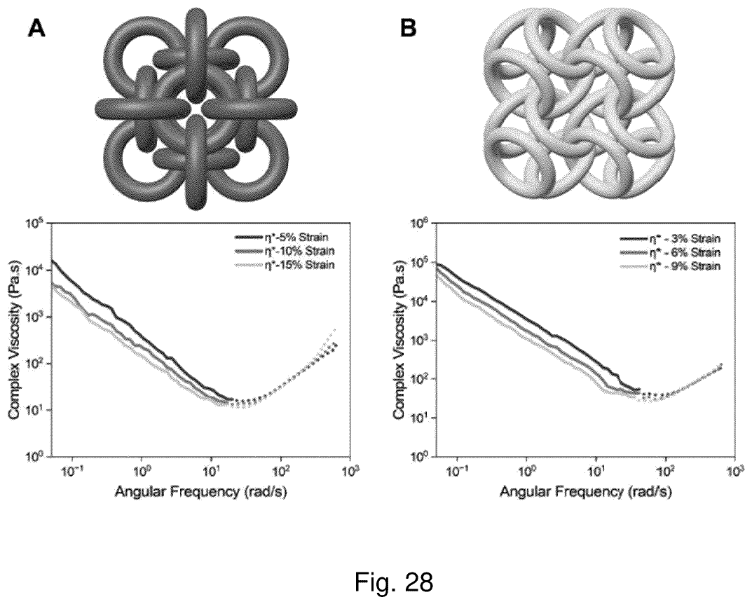

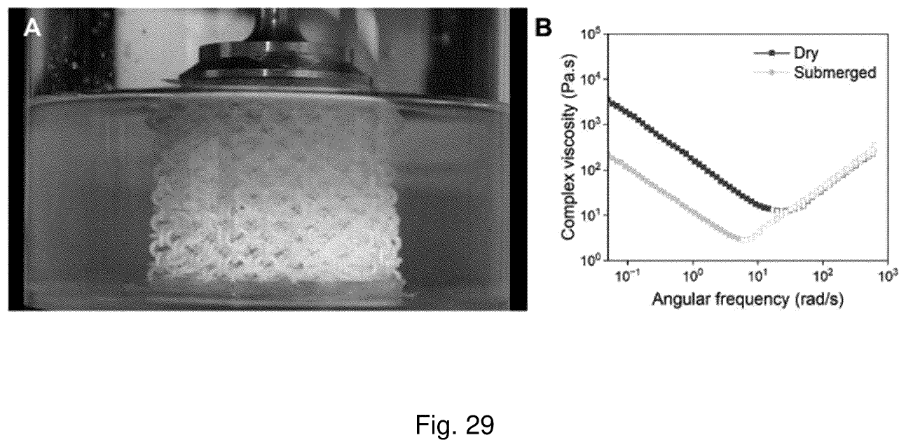

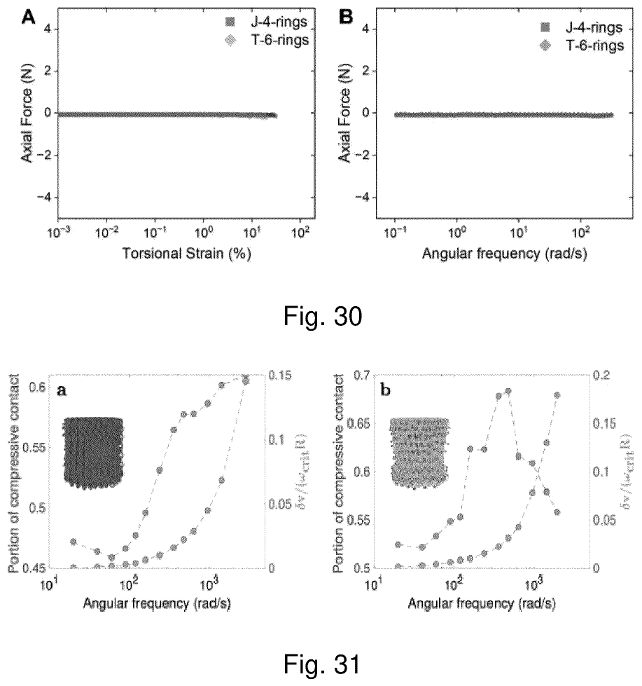

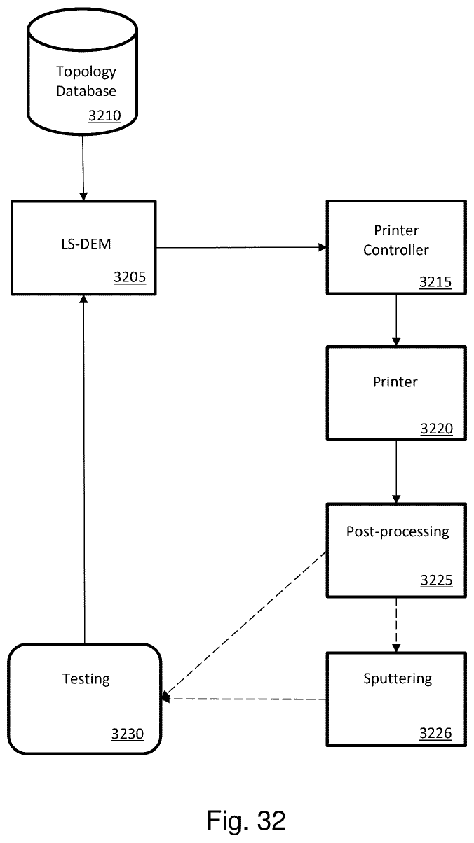

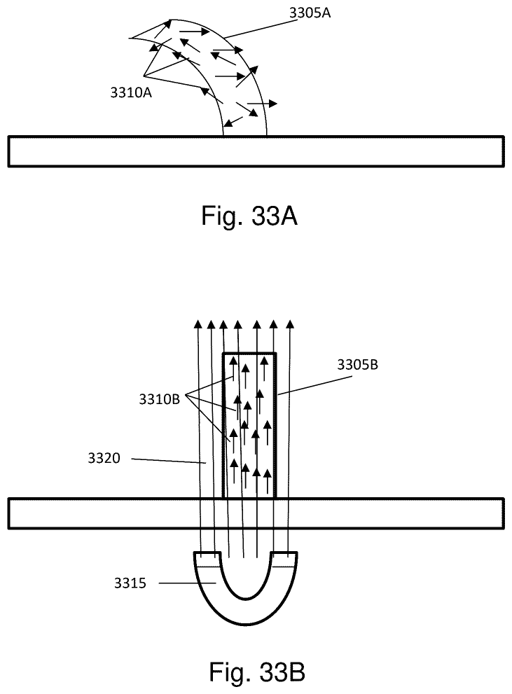

Unlike other architected materials, PAMs exhibit a notable ability to adjust their interparticle arrangements in response to external loads, a characteristic also found in granular systems. This particles' rearrangement leads to two mechanical regimes, which appear under all deformation modes: (i) a fluid-like response, with a vanishing shear modulus, linked to relative particles' motion; and (ii) a solid-like response, characterized by particles' deformation, beyond the jamming transition. To describe the fluid-like mechanical response, simple shear and rheology tests on J-4-ring and T-6-ring samples (FIG. 4, panels A, B) can be conducted. These PAMs were selected because they are composed of toroidal particles, which have greater kinematic DOFs. To control testing conditions, fabricate samples that incorporated top and bottom gripping plates (FIG. 4, panels A, B, FIG. 11).

Under shear loads, both J-4-ring and T-6-ring samples demonstrated a plateau region with force values close to zero, indicative of a fluid-like behavior (FIG. 4, panels C, F). Beyond a critical strain, such plateau then transitioned to a quasi-linear elastic region, typical of solid-like behavior. This transition can be correlated to the reduced DOFs between rings, which jam under external tensile, compressive, or shear loads. The fluid-like and solid-like regimes can be programmed by designing the catenation topologies and particle geometries.

To further understand this fluid-solid duality, characterize the rheological properties of cylindrical shaped PAM samples (FIG. 4, panel B). In oscillatory amplitude sweep experiments (FIG. 4, panels D, G), the J-4-ring and T-6-ring samples initially displayed a decrease in storage modulus (G′), loss modulus (G″), and complex viscosity (η*) with increasing torsional strain. Upon reaching the critical jamming strain observed in simple shear tests, all three parameters begin to increase [43]. In the oscillatory frequency sweep experiments (FIG. 4, panels E, H), both J-4-ring and T-6-ring samples exhibited an unusual inflection in viscosity at high angular frequency. When subjected to torsional strains below their respective jamming transition thresholds, a notable transition from shear-thinning to shear-thickening was observed with increasing oscillation angular frequency. In the latter part of the shear-thickening phase, the substantial increase in viscosity is likely influenced by the inertia effects of particles under high-frequency oscillation conditions. Although both shear-thinning and shear-thickening behaviors have been observed in various materials [43] [44], they have not been reported concurrently in the same material, particularly with the observed pronounced reduction in viscosity followed by a substantial increase, as a function of both angular frequency or torsional strain.

To better understand this unusual frequency-dependent thinning-to-thickening transition, model the response of PAMs using the LS-DEM. The focus is on modeling the rheological experiments, and the inflection of η* with increasing angular frequency. Because the mechanical deformation of each particle is minimal compared to the translation observed in their rigid body motion (e.g., rearrangement) in the experiments, model the rings as rigid particles. Here, LS-DEM can be used to model sweep experiments for both J-4-ring and T-6-ring samples. Construct digital twins, replicating the ring's shape, density, size, and spatial arrangement, as well as the total number of rings in each respective sample.

All simulations qualitatively capture the inflection of η* observed in experiments (compare FIG. 4, panels I, K with FIG. 4, panels E, H). These simulations also agree with experiments in that the value of η* at the inflection point is smaller for the J-4-ring sample compared to that for the T-6-ring sample. However, the simulations overestimate the angular frequency at which the inflection of η* happens. This mismatch could be due to imprecisions in the 3D printed particle geometry, contacts' imperfections, and the presence of friction, which are not included in these models. These discrepancies can also lead to considerable differences in the sample's packing structure in the relaxed configuration in experiments and simulations, which can shift the angular frequency at which the inflection of η* happens. Nevertheless, these simulations provide particle-scale details that allow one to better understand the mechanisms underpinning this thinning-to-thickening transition. More specifically, from the point view of the rheophysics of dense granular materials [45]-[47], η* takes contribution from two components: a “contact” component (which corresponds to percolating and enduring force chains in the statics of granular media, used often in the soil mechanics community), and a “fluctuation” component (which corresponds to the degree of turbulency of granular flow stemming from short-lived particle collisions, used often in the fluid mechanics community).

In these experiments, as the excitation frequency increases, it is expected that both samples experience a transition from a contact-dominant (or “quasi-static”) regime to a fluctuation-dominant (or “inertia”) regime. As such, the initial decrease of η* may be understood as the decrease of contact (due to stronger centrifugal effects) in the contact-dominant regime, while the later increase of η* can be understood as the increased degree of “turbulency” (due to a faster external excitations) in the fluctuation-dominant regime. This concept is confirmed by looking at FIG. 4, panel I (inset), which shows, for the simulation of the J-4-ring sample, the variation of the average contact number per particle, and of the normalized average particle velocity fluctuation, δv (computed as the square root of granular temperature [45], see Numerical Simulation of Rheological Experiments herein for the calculation procedure), as functions of the angular frequency. As the angular frequency increases, the average contact number per particle decreases while the particle velocity fluctuation increases. In particular, the latter shows a much rapid increase rate once exceeding the frequency of the inflection point of η*, which suggests the transition from the contact-dominant regime to the fluctuation-dominant regime. A similar observation can be made for the T-6-ring sample, by looking at FIG. 4, panel K (inset) and comparing it to FIG. 4, panel I (inset). According to kinetic theory [48], the viscosity of granular flow, η, depends on the density and granular temperature through a scaling η~(ρ_{c}−ρ)^{-α}δv, where ρ_{c} is the material density at random close packing, p is the material density at a given flow state, and a is a scaling exponent whose value depends on particle properties (such as shape and surface friction).

The relevance of this theory can be tested in describing the rheological response of PAMs. Due to difficulties in calculating objectively ρ for the simulated PAM samples, use the mean contact number [symbol] Z [symbol] in place of ρ (with Z_{c} the contact number at random close packing), assuming a power-law scaling between ρ and [symbol] Z [symbol] can translate from conventional granular materials to PAMs. FIG. 4, panels J and L show the variation of the normalized viscosity,

$$\frac{{η}^{*}}{δ v}$$

as a function of Z_{c}− [symbol] Z [symbol] for the J-4-ring sample and the T-6-ring sample, respectively. One observes that in the low excitation frequency regime (before the inflection of (1), the rheological response follows a power-law scaling (black dashed line in FIG. 4, panels J,L) as predicted by kinetic theory. However, as progressing into the high frequency domain (after the inflection of (1), the rheological response deviates further away from the respective power-law scaling (insets of FIG. 4, panels J, L). The exact reason for this deviation is unknown, but one possible cause is the breakdown of the power lawing scaling between [symbol] Z [symbol] and ρ in the high frequency domain due to the presence of tensile contact (for preventing particles from separating) that is absent in conventional granular materials. Lastly, it is noted that, Z_{c} is unknown for PAMs. For results shown in FIG. 4, panels J, L, use the mean contact number of the sample before torsion experiment (that is, after gravity settlement), as an estimation of Z_{c}. A different Z_{c} will change the scaling exponent presented in FIG. 4, panels J, L, but it will not change the observed power-law relation.

### Example 5: Programmable Critical Jamming Strains

The role of particle geometry and particle's linking topology is involved with the jamming transition in PAMs. The local catenation topologies in PAMs play a pivotal role in defining their mesoscale (e.g., cell-to-cell, layer-by-layer) DOFs, which in turn dictates the critical strain for jamming

$${ϵ}_{c}^{*} :$$

critical compressive jamming strain;

$${γ}_{s}^{*}$$

critical shear jamming strain). For instance, J-4-ring PAMs show a substantially higher

$${γ}_{S}^{*}$$

as compared to T-6-ring PAMs. whereas T-6-ring exhibits a higher

$${ϵ}_{c}^{*}$$

relative to J-4-ring. In J-4-ring PAMs, shear loading induces a coordinated rearrangement of particles, thereby amplifying

$${Y}_{S}^{*}$$

(FIG. 5, panel A).

During this process, rings oriented parallel to the shearing direction maintain their orientations, while those perpendicular to the shearing direction rotate in a coordinated manner, facilitating a greater

$${Y}_{S}^{*} .$$

However, under compression or tension, the rings' inability to adjust their positions—restricted by limited DOFs from neighboring particles-results in a reduced

$${ϵ}_{c}^{*} .$$

In contrast, T-6-ring PAMS rely on a ‘scissor’ mechanism FIG. 5, panel C): Upon compressive loading, all rings adjust their orientations coordinatively, which allows for a larger

$${ϵ}_{c}^{*} .$$

In addition to the choice of catenation topology, the thickness, d, of the torus substantially influences the jamming transition in PAMs (FIG. 5, panel D). A decrease in d generally correlates with an increased DOF in PAMs. To elucidate this relationship, fabricate a series of J-4-ring and T-6-ring PAMs with constant ring diameters (D) but varied d. Measure the critical jamming strain under both shear

$$( {γ}_{s}^{*} )$$

and compressive

$$( {ϵ}_{c}^{*} )$$

loading conditions as a function of thickness d (FIG. 5, panel B). Regardless of the catenation topology, reductions in d are associated with increases in both γₛ and

$${ϵ}_{c}^{*} .$$

Furthermore, it is observed that catenation topology significantly impacts the predominant deformation modes. For instance, the

$${Y}_{S}^{*}$$

for J-4-ring PAM (FIG. 5, panel D), which exhibit the largest d/D ratio and therefore the lowest DOF, is higher compared to T-6-ring PAMs (FIG. 5, panel O), which have a much lower d/D ratio.

### Example 6: Length-Scale Dependence and Electrostatic Reconfiguration of μ-PAMs

The deformability of PAMs is primarily influenced by their particle geometries and the particle-to-particle DOF, which are expected to be scale independent. Hence, reducing the particle size by a factor of ca. 60 will retain the characteristic mechanical response of macroscopic systems, such as their quasistatic compressive behavior and liquid-fluid duality. To validate this hypothesis, fabricate PAMs using two-photon lithography with post-printing oxygen plasma etching [37]. Upon completion of the fabrication and release process—where plasma etching removes thin support materials required during fabrication-the-PAMs demonstrated a gravitational relaxation analogous to that observed in their macroscopic counterparts (FIG. 23). Then design a series of C-6-TT PAMs (as in FIG. 15) varying their volume fractions, by changing the beam thicknesses of the particles. Following an increasing order of beam thickness, label the PAMs as I, II, and III (FIG. 15). The same PAMs were fabricated at both macroscale (Ω_I, Ω_II, Ω_III) and microscale (μ_I, μ_II, μ_III), scaling them by a factor of 60 in all dimensions (i.e., sample side lengths: 24 mm and 400 μm). Due to the differences in fabrication methods, use slightly different acrylic polymers for the macro- and micro-scale samples. Nevertheless, qualitative agreement is found between the mechanical responses of PAMs across scales (FIG. 6, panels A-C). The energy absorption capacities of all C-6-TT PAMs were calculated by integrating the areas under the stress-strain curves. The experiments reveal that scale factors (U_{μ}/U_{Ω}) among all I, II, III designs to be near constant of 12.76+0.53 (FIG. 6, panel A). One defining difference between micro-scale and macro-scale PAMs is their dramatically different surface-to-volume ratio (~60 times larger in the—PAM samples) and the reduced weight of each particle in the catenated network (~216,000 times lower in the μ-PAM samples). Such discrepancy can be exploited to observe the role of inter-particle forces (e.g., electrostatic repulsion) in the global deformation of PAMs. An estimated threshold size for this micro-scale actuation is a feature size or sample side length of less than or equal to 1 mm, with preferable ranges being less than or equal to 500 μm, and more preferably less than or equal to 200 μm. This actuation was demonstrated with an exemplar having a sample side length of approximately 0.40 mm (400 μm).

To test this hypothesis, tessellate J-4-ring μ-PAM voxels to form various geometries: a side-anchored cube (FIG. 6, panel D), a point-anchored cube (FIG. 6, panel E), a bottom-anchored numeral ‘1’ (FIG. 6, panel F), and a bottom-anchored letter ‘T’ (FIG. 5G). Then coat each—PAM sample with a thin layer of copper, approximately 300 nm in thickness, to provide electrical conductivity. Then position the samples atop a Van de Graaff generator with direct electrical contact (FIG. 23). As electrostatic charges accumulate, the individual rings within the—PAMs began to repel each other, due to increased electrostatic repulsion. This electrostatic interaction prompted the PAMs to both expand outward in all directions due to inter-ring repulsion and elongate upwards due to repulsion between the μ-PAMs and the substrate against gravity, transforming each initially collapsed structure into a structurally deployed state (FIG. 6, panels D-G). The charged μ-PAMs will stay in this deployed geometry until the Van de Graaff generator is discharged by neutralizing the electrical charge stored in its metallic dome, to which the conductive substrate of the PAM samples is attached. The transition between the uncharged, compact state and the charged, deployed state was fast (<0.1 s) and completely reversible. This behavior is derived from the fluid-solid duality also observed in the macroscale samples. Global deformations are allowed by the intrinsic DOF, but constrained when reaching the tensile critical jamming strains, resulting in a ‘lockable’ 3D shape-morphing behavior. This suggests that μ-PAMs driven by electrostatic forces can be engineered as responsive elements in remotely actuated materials, for micro-scale devices and smart material systems.

### Example 7: Structural Turnabilities of PAMS

By changing the particles shapes within similar symmetries (like from octahedra to cuboctahedra) within a given topology (FIGS. 8A-8B), the locking mechanisms can be altered (e.g., transitioning from a corner-to-corner linking, as in a J-4-OCT to an edge-to-edge interlocking in the J-4—CO), thus varying the DOFs between particles (FIGS. 8A-8B). Reducing the particles' thickness, thereby decreasing the volume fraction, typically increases the kinematic DOFs between units within the same global topology and the resulting mechanical response of the PAMs.

To explore a broader property space of PAMs, three orthogonal tuning methods have been identified: volume fraction tuning, geometry tuning, and topology tuning. Each method allows for precise control over the mechanical properties and responses of PAMs, enabling a wide range of potential applications.

Volume Fraction Tuning. The mechanical characteristics of PAMs, such as critical jamming strains and moduli, can be significantly influenced by adjusting the volume fraction of the materials. This is achieved by varying the beam thicknesses (d) of the particles, whether they are polyhedral, polygonal, or torus-shaped. Increasing the beam thickness results in a higher volume fraction, which typically leads to smaller critical jamming strains (FIG. 5, panel B) and larger moduli (FIG. 6, panel A).

Particle Geometry Tuning. For a given crystalline network topology, an infinite variety of particle geometries can be designed. For instance, torus particles can be replaced with polygonal particles (FIG. 10, panels A-D), resulting in similar (FIG. 10, panels A, B) or distinct (FIG. 10, panels D, E) mechanical behaviors. Additionally, polyhedral particles can be partially truncated while maintaining their symmetries, altering the local interlinking mechanisms among corner-to-corner, face-to-face, and edge-to-edge locking (FIGS. 8A-8B). By fine-tuning these kinematic degrees of freedom (DOFs), one can achieve vast turnability in both solid-like and fluid-like mechanical properties.

Catenation Topology Tuning. The mapping-based design strategy allows for flexible adaptation of catenation topologies. Either singular or multiple particles can be mapped onto a node in a network topology, resulting in varied catenation structures (FIG. 2, panel D). This flexibility enables the fine-tuning of catenation topologies by substituting a single polyhedral particle with a cluster of spatially arranged planar particles (FIGS. 8A-8B), provided they share the same symmetry. Such substitutions increase the kinematic DOFs of the resulting PAMs, as planar particles can move relative to each other.

Consequently, these PAMs can exhibit drastically different mechanical behaviors, enhancing their adaptability and performance across various applications. Collectively, these tuning methods empower the design of PAMs with tailored mechanical properties, paving the way for innovative solutions in stimuli-responsive materials, energy-absorbing systems, and morphing architectures.

### Example 8: PAMs' Behavior Under Uniaxial Compression

Repeated loading and unloading cause PAMs to develop a permanent residual strain. Under cyclic compression at lower strains, PAMs show a reduced peak stress and a hysteretic response with a progressively smaller area, leading to less energy being dissipated in each cycle (FIG. 13). Most of this reduction happens within the first few cycles, after which the PAMs stabilize into a steady-state response. This initial drop in energy dissipation, known as preconditioning, is a common characteristic of many rubbery and biological materials. Under cyclic compression with progressively increasing strain (from 10% to 50%), the material continues to follow the primary loading path until extensive damage occurs, as long as the strain in each cycle exceeds the maximum strain of the previous cycle (FIG. 12). These observations mirror the Mullins effect seen in certain rubbers [53].

T-6-ring and T-6-HEX exhibit comparatively lower stiffness and can withstand maximum strain without damage to their constituent units (FIG. 12, panels A, B), unlike PAMs composed of polyhedral particles (FIG. 12, panels C, F). This discrepancy can be attributed to the fact that jamming in polyhedron-based PAMs occurred at lower strain values than in 2D polygons or rings (FIG. 12, panels A-F). Additionally, the larger number of kinematic DOFs and the coupled deformation modes of rings and hexagonal particles resulted in lower overall PAM's stiffness. This phenomenon is reminiscent of powders, where sphere-like particles (analogous to PAMs composed of polyhedral particles) are found to exhibit higher compressive strength and elastic modulus, as compared to flake-like particles (analogous to T-6-ring and T-6-HEX). Concurrently, it is also possible to create PAMs with polygonal particles exhibiting higher stiffness, akin to PAMs composed of polyhedral particles, by adjusting the kinematic DOFs. For example, J-4-ring and J-4-square structures demonstrated relatively higher stiffness (FIG. 12, panels D, E). Therefore, PAMs can be engineered to achieve greater stiffness by either (i) increasing the number of edges of the particles or (ii) disrupting the kinematic DOFs among particles.

### Example 9: Anisotropy in PAMs and their Mechanical Behavior

PAMs can inherit intrinsic anisotropies from their crystalline network-based designs. One common manifestation of this anisotropy is the orientation-dependent wetting behavior observed when PAMs are fabricated into spherical shapes. Under gravity, the structural outlines of PAMS will depend on their orientation relative to their crystalline axes (FIGS. 3A-C). This anisotropic behavior is markedly different from that of traditional solids, fluids, or granular media, providing PAMs with unique adaptive capabilities.

To better quantify the impact of structural anisotropy on the mechanical behavior of PAMs, a series of simple shear experiments can be performed on J-4-ring PAM samples exhibiting varying degrees of structural anisotropy. Specifically, the orientation of cubic boundary conditions can be manipulated relative to the periodic lattices by rotating them 15, 30, and 45 degrees (FIG. 26). Owing to the symmetry properties of the J-4-ring PAM, a rotation of 15 degrees corresponds to an effective rotation of 75 degrees, while a 30-degree rotation corresponds to 60 degrees. During the simple shear experiment, the plane of structural rotation was designated as the plane for y-direction shear, with the orthogonal plane corresponding to x-direction shear. At zero degrees of rotation, the structure exhibited symmetry about the axis perpendicular to the shearing direction on the shear plane, resulting in equal displacements in the positive and negative y-directions at near-zero shear force. As the rotation increased to 15 and 30 degrees, this symmetry was disrupted, leading to more pronounced anisotropic behavior and variations in the displacements observed.

At 45 degrees of rotation, the inherent orthotropic symmetry of the structure's topology reinstated the symmetry, producing displacements in both directions similar to those at zero degrees, but with different displacement values, all corresponding to near-zero shear force. Since the structural rotation was confined to the y-direction shear plane, the symmetry on the x-direction shear plane remained unaltered. Consequently, the structure exhibited identical displacements in the positive and negative x-directions at near-zero shear force, independent of the applied rotation. These displacement values showed only minimal variation with increasing rotation, underscoring the invariable response of the x-direction shear despite the structural changes occurring in the y-shear plane.

### Example 10: Randomness and Anisotropic of PAMs' Mechanical Behavior

One distinguishing characteristic of PAMs is their remarkable ability to adapt to environmental conditions, including external forces. This adaptability makes their global shapes and mechanical behaviors highly sensitive to their initial configurations. This effect typically leads to random mechanical responses to identical loading conditions.

Due to the infinite possible ways PAMs can adapt their particle arrangements, their mechanical behaviors, such as responses to compressive loads, are highly sensitive to the nuances of their initial configurations. For example, a cubic J-4-ring PAM sample subjected to the same compressive loading condition ten times, but with slightly different initial configurations each time, can exhibit varying moduli despite similar loading-unloading profiles (FIG. 14). Notably, this limited degree of randomness arises from the complex interplay between initial particle arrangements and mechanical responses.

### Example 11: Rheology Test

Rheology is the study of how materials flow and deform under applied forces, focusing particularly on the behavior of complex fluids and soft solids. This discipline examines the responses of materials to stress, strain, and time, thereby revealing intricate dynamics related to flow and deformation. Such insights are pivotal in understanding material properties such as viscosity, elasticity, and plasticity. In this context, several key rheological terms are relevant:

Complex modulus (G*) In oscillatory tests, G is defined as the ratio of the applied (or measured) stress amplitude to the measured (or applied) strain amplitude. G* serves as a precise quantitative indicator of a material's ability to resist deformation.

Phase angle (8) Phase angle is a measure of the balance between viscous and elastic behaviors in a material, ranging from 0° (indicative of a purely elastic material) to 90° (characteristic of a fully viscous material).

Storage modulus (G′)—Defined by the equation G′=G*cos (δ), this modulus quantifies the elastic component of a material's viscoelastic behavior, indicating how much energy is stored and recovered in one cycle of deformation.

Loss modulus (G″)—This modulus is defined by the formula, G″=G*sin (δ), this modulus reflects the viscous component of a material's viscoelastic behavior, representing the energy dissipated as heat within the material during deformation.

Complex viscosity (η*)—This parameter evaluates the overall resistance a material presents to flow under oscillatory conditions. It is dependent on the angular frequency (ω) and is calculated using the equation η*=G*/ω. Complex viscosity effectively encapsulates both the viscous and elastic responses of the material, providing a comprehensive measure of how the material behaves under dynamic stresses.

In the case of this study, the storage modulus (G′) indicates the energy stored as the deformation of PAMs, while the loss modulus (G″) reflects the energy dissipated through internal friction within PAMs during the test. A material exhibits characteristics of a viscoelastic solid when G′>G″, indicating predominant elastic behavior. Conversely, it behaves as a viscoelastic fluid G′<G″, where viscous behavior is more pronounced. The tests confirm that PAMs behave as viscoelastic solids, as evidenced by consistently higher G′ than G″ in all oscillatory tests, including oscillatory amplitude and frequency sweeps.

All rheology tests of PAMs showed varying G′ and G″, as well as η*, exhibiting a transition from shear-thinning to shear-thickening fluid behavior. During the amplitude and frequency sweeps, both the moduli and viscosity initially decrease and then increase as the shear rate increased (FIG. 4). This distinctive response is attributed to the semi-granular/liquid-like nature of PAMs led to complex interactions between constituent particles, resulting in this unique rheological behavior. In contrast, when the same amplitude sweep tests were performed on a simple cubic truss lattice with the same material, manufacturing technique, and outlining dimensions as PAMs, both moduli and complex viscosity remain constant regardless of increase in shear rate (FIG. 22), demonstrating a typical behavior of a solid.

### Example 12: Non-Newtonian Behavior

Various PAM configurations have marked shear-thinning and shear-thickening behavior to a degree beyond previous structures (e.g. 2D sheet design) typically show.

For example, the J-4-ring configuration shows:

Shear-thinning: The complex viscosity (η*) decreases from approximately 10\hat{ }5 Pa·s (at 0.1 rad/s) down to a minimum of approximately 10\hat{ }2 Pa·s (at ~100 rad/s)

Shear-thickening: After its minimum, the viscosity increases from 10\hat{ }2 Pa·s back up to 10\hat{ }3 Pa·s as the frequency approaches 1000 rad/s.

As another example, the T-6-ring configuration shows:

Shear-thinning: The complex viscosity decreases from approximately 10\hat{ }5 Pa·s (at 0.1 rad/s) down to a minimum just below 10\hat{ }3 Pa·s (at ~100 rad/s).

Shear-thickening: After its minimum, the viscosity increases from below 10\hat{ }3 Pa·s up to approximately 10\hat{ }4 Pa·s as the frequency approaches 1000 rad/s.

See e.g. FIGS. 24 and 28.

In addition to the non-Newtonian behavior, several other quantifiable attributes distinguish these 3D polycatenated architected materials from conventional 2D lattices or bonded 3D trusses. The 3D PAMs exhibit a reversible near-zero shear plateau at finite strain before jamming, a feature that is absent in bonded trusses. The materials also demonstrate sustained deformation without catastrophic layer failure under oscillatory loading. This is in contrast to a simple cubic truss, which was observed to auto-stop from catastrophic failure at approximately 5.6% strain in an amplitude sweep test. Furthermore, the 3D structures exhibit orientation-dependent gravity relaxation outlines in 3D volumes, as shown in FIG. 3A-C. Finally, the energy absorption is tunable by topology and volume fraction, and these properties show consistent scaling trends across different scales, as shown in FIG. 6A.

### Example 13: Submerged vs. Dry Rheology

To understand environmental effects, the rheological behavior of a J-4-ring PAM was tested in both a dry state and when fully submerged in water (FIG. 29). The results indicate that the complex viscosity profiles remain qualitatively similar, both showing the characteristic shear-thinning followed by shear-thickening. However, the presence of water as a lubricant appears to slightly alter the particle-particle interactions, affecting the precise viscosity values and the inflection point. This demonstrates the potential to modulate the material's rheological response by controlling the surrounding fluid environment.

### Example 14: Friction-Coefficient Sensitivity in Simulation

The numerical LS-DEM simulations were used to investigate the sensitivity of the material's rheological behavior to inter-particle friction (FIG. 24). By varying the friction coefficient in the simulation, it was observed that this parameter has a significant influence on the complex viscosity and the overall thinning-to-thickening transition. This sensitivity confirms that friction at the particle-particle contacts is a key mechanism governing the bulk response and highlights the importance of accurately modeling or tuning surface properties to achieve a target rheological profile.

### Example 15: Material and Process Variants

To demonstrate the versatility of the PAM design framework, structures were fabricated using varied material compositions and additive manufacturing processes (FIG. 25). Beyond the acrylic polymers used for primary testing, PAMs were successfully fabricated using Nylon, Thermoplastic Polyurethane (TPU), and metal. These materials were processed using methods such as Selective Laser Sintering (SLS) and Selective Laser Melting (SLM). Each combination of material and process results in different macroscopic properties, showcasing that the polycatenated topology is a scale- and material-independent design strategy.

### Example 16: Multi-Stability and Training of J-4-square

A J-4-square PAM demonstrates a specific case of multi-stability. This structure was observed to have two stable configurations: an expanded, stiff configuration (denoted ‘L’) and a collapsed, flexible configuration (denoted ‘S’). The transition between these ‘L’ and ‘S’ states can be induced via an auxetic mechanism, similar to a “rotating squares” behavior. Furthermore, this transition can be trained by the cyclic application of external loading, allowing the material to be set into a desired stable state.

### Example 17: Closed-Loop Design and Fabrication Workflow

FIG. 32 illustrates a tangible, computer-implemented workflow for engineering and fabricating a 3D polycatenated architected material with a specific target property, following the process of: (1) Compute/Design, (2) Generate Build Files, (3) Fabricate/Coat, and (4) Measure/Update.

(1) Compute/Design: A designer wishes to create a micro-scale structure for a soft-robotics application that requires high shear flexibility. Using the system for engineering a structure, the designer selects a J-4-ring topology (known for shear flexibility) and a toroidal particle body geometry from a database(s) (3210). They set an initial d/D ratio of 0.25. The system's contact-aware simulation module (e.g. LS-DEM) (3205) is used to compute a predicted critical shear jamming strain

$$( {γ}_{s}^{*} ) ,$$

which is reported to the user Interface as 35%.

(2) Generate Build Files/Toolpaths: If, for example, the designer determines that 35% is not flexible enough, they can iterate in the “compute” phase, changing the d/D ratio to 0.15. The simulation module re-computes and now predicts a

$${γ}_{s}^{*}$$

of 65%. Satisfied, the designer finalizes the design. The system's parametric modeling engine is then used to generate a concrete build file (e.g., an .stl file) for the entire interlocked structure. The system also generates the associated process parameters and toolpaths required for the target manufacturing device, such as the precise exposure paths and laser power settings for a two-photon lithography (2PL) printer.

(3) Fabricate/Coat: The generated build file and toolpaths are sent to a controller (3215) communicatively coupled to the manufacturing device (e.g. 2PL printer) (3220). The printer then fabricates the micro-scale J-4-ring structure. Following fabrication, the structure undergoes post-processing (3225), including a plasma etch to remove all support material and free the interlocks. If the part is intended for actuation, it is then moved to a sputtering system to apply a conductive coating (e.g., 300 nm of copper) (3226).

(4) Measure/Update: The physical, coated micro-structure is measured. It is subjected to mechanical couponing using a micromechanical testing system (3230) to determine its actual mechanical properties. The experimental data reveals the true

$${γ}_{s}^{*}$$

is 62%. The new experimental data is fed back into the system (3205) for engineering a structure. The simulation module uses this data to update and calibrate its digital twin model, refining its predictive accuracy for future design cycles.

This demonstrates a complete, closed-loop process where the digital design is directly linked to physical fabrication, and the results from the physical part are used to improve the digital model.

### Example 18: Magnetic Field Actuation (Prophetic)

A structure is engineered using a J-4-ring topology, similar to that described in Example 6, but at a macro-scale. FIGS. 33A and 33B show an example of such a structure. The plurality of unit particles are fabricated via selective laser sintering (SLS) from a composite material, comprising a polymer (e.g., Nylon) blended with ferromagnetic microparticles (e.g., iron oxide). In its first state (FIG. 33A), absent a magnetic field, the structure (3305A) exhibits its characteristic fluid-like regime and high flexibility, due to the ferromagnetic particles having random magnetic field directions (3310A). The structure (3305B) is then (FIG. 33B) placed within a magnetic field, such as from a controllable electromagnet (3315). When a current is applied, generating a uniform magnetic field (3320), the ferromagnetic-doped particles experience a torque, causing them to align (3310B) with the magnetic flux lines (3320).

This collective alignment constrains the relative rotation and kinematic degrees of freedom between the interlocked particles. This constraint causes the bulk material to convert from its first flexible state to a second, significantly stiffer, ‘jammed’ state. This conversion is reversible upon removal of the magnetic field. This demonstrates a method for actively and reversibly tuning the material's stiffness, for example, for use in adaptive vibration damping or haptic feedback systems.

### Example 19: Thermal Change Actuation (Prophetic)

A structure is engineered with a T-6-ring topology, designed with a high d/D ratio to have a low critical jamming strain, making it relatively stiff in its as-fabricated state. The structure is fabricated using two-photon lithography from a Shape Memory Polymer (SMP) resin.

After fabrication, the structure is heated above its glass transition temperature (Tg), mechanically deformed into a temporary “compact” and collapsed state, and then cooled below its Tg to lock in this temporary state. This forms the first state. The structure can be stored or integrated into a device in this compact state.

To trigger actuation, the structure is heated again (e.g., via ambient heating or an integrated resistive heater) above its Tg. The SMP material releases its stored strain, causing the structure to “self-fold” and expand, converting from its first compact state to its second, permanent “deployed” state. This one-way deployment demonstrates a method for creating deployable systems, such as in medical implants or aerospace components, that can be shipped in a compact form and actuated on demand.

### Example 20: Environmental Actuation (Prophetic)

A structure is fabricated at the micro-scale using a high-resolution 3D printing process. The constituent material is a “solvent-responsive polymer” (a hydrogel) that is known to swell significantly in the presence of water. In its first state (dry), the structure is compact and its particles have minimal clearance, keeping it in a jammed, stiff state. When the structure is exposed to a solvent (water) or high humidity, the hydrogel absorbs the liquid and swells.

This swelling increases the volume of the structural members, exhausting all remaining clearances and causing the structure to “lock” into a rigid, solid-like state. In a different configuration, a material is chosen that is a “light-responsive polymer” containing azobenzene moieties. Upon exposure to UV light (the trigger), the polymer chains contract, causing the particles to shrink or fold, converting the structure from an “expanded” first state to a “collapsed” second state.

This process is reversible by exposing the structure to visible light. This demonstrates a method for creating environmentally-aware or optically-controlled actuators.

In summary, embodiments described herein are 3D polycatenated architected materials, systems, and methods. The material comprises discrete unit particles mechanically interlocked to form a 3D network patterned after a crystalline topology. This structure provides a tunable dual mechanical response, transitioning from a fluid-like regime to a solid-like regime at a programmable “critical jamming strain.” Methods are disclosed for designing the material using a closed-loop digital twin, manufacturing the material via additive processes, and preconditioning the material. Methods and systems for actuating the material using mechanical, electrostatic, magnetic, thermal, or environmental triggers are also disclosed. Further disclosed are kits-of-parts for assembling the material, and end-user systems, such as impact protection systems, soft robotics, and medical implants, incorporating the material.

in particular described the material comprises discrete unit particles, each having a particle body with structural members and interlocking openings at nodes. The particles are mechanically interlocked via inter-layer linkages, with a structural member of one particle passing through an opening of an adjacent particle, to form a 3D network patterned after a crystalline topology. This structure provides a tunable dual mechanical response: a fluid-like regime with near-zero shear modulus transitions to a solid-like regime with nonlinear stress at a programmable “critical jamming strain.” The jamming strain is programmed by selecting structural variables including catenation topology, particle body geometry, and structural member dimensions. Methods for designing the material using a digital twin, which may be calibrated in a closed-loop process using measurement data from a physical structure, manufacturing the material via additive processes are described. Method for actuating the material are also described, such as actuation of micro-scale, conductively-coated versions of the material using electrostatic charges or actuation using constituent materials responsive to magnetic fields, thermal changes, or environmental stimuli.

The examples set forth above are provided to give those of ordinary skill in the art a complete disclosure and description of how to make and use the embodiments of the 3D polycatenated architecture material and related compositions, devices, methods and systems of the disclosure, and are not intended to limit the scope of what the Applicants regard as their disclosure. Modifications of the above-described modes for carrying out the disclosure can be used by persons of skill in the art and are intended to be within the scope of the following claims.

The entire disclosure of each document cited (including patents, patent applications, journal articles including related supplemental and/or supporting information sections, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by preferred embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible sub-combinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified can be employed in the practice of the disclosure without resort to undue experimentation. All art-known functional equivalents, of any such methods, device elements, and materials are intended to be included in this disclosure.

Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein can be excluded from a claim of this disclosure. The disclosure illustratively described herein suitably can be practiced in the absence of any element or elements, limitation or limitations, which is not specifically disclosed herein.

“Optional” or “optionally” means that the subsequently described circumstance can or cannot occur, so that the description includes instances where the circumstance occurs and instances where it does not according to the guidance provided in the present disclosure. Combinations envisioned can be identified in view of the desired features of the device in view of the present disclosure, and in view of the features that result in the formation.

A number of embodiments of the disclosure have been described. The specific embodiments provided herein are examples of useful embodiments of the disclosure and it will be apparent to one skilled in the art that the disclosure can be carried out using a large number of variations of the devices, device components, methods steps set forth in the present description. As will be obvious to one of skill in the art, methods and devices useful for the present methods can include a large number of optional composition and processing elements and steps.

In particular, it will be understood that various modifications may be made without departing from the spirit and scope of the present disclosure. Accordingly, other embodiments are within the scope of the following claim.

### REFERENCES

ADDIN Mendeley Bibliography CSL_BIBLIOGRAPHY 1. A. J. D. Shaikeea, H. Cui, M. O'Masta, X. R. Zheng, V. S. Deshpande, The toughness of mechanical metamaterials. Nat. Mater. 21, 297-304 (2022).
2. X. Zheng, H. Lee, T. H. Weisgraber, M. Shusteff, J. DeOtte, E. B. Duoss, J. D. Kuntz, M. M. Biener, Q. Ge, J. A. Jackson, Ultralight, ultrastiff mechanical metamaterials. Science 344, 1373-1377 (2014).
3. R. Xue, X. Cui, P. Zhang, K. Liu, Y. Li, W. Wu, H. Liao, Mechanical design and energy absorption performances of novel dual scale hybrid plate-lattice mechanical metamaterials. Extrem. Mech. Lett. 40, 100918 (2020).
4. K. Davami, L. Zhao, E. Lu, J. Cortes, C. Lin, D. E. Lilley, P. K. Purohit, I. Bargatin, Ultralight shape-recovering plate mechanical metamaterials. Nat. Commun. 6, 10019 (2015).
5. O. Al-Ketan, A. Soliman, A. M. AlQubaisi, R. K. Abu Al-Rub, Nature-inspired lightweight cellular co-continuous composites with architected periodic gyroidal structures. Adv. Eng. Mater. 20, 1700549 (2018).
6. O. Al-Ketan, R. K. Abu Al-Rub, Multifunctional mechanical metamaterials based on triply periodic minimal surface lattices. Adv. Eng. Mater. 21, 1900524 (2019).
7. J.-F. Ganghoffer, A. Wazne, H. Reda, Frontiers in homogenization methods towards generalized continua for architected materials. Mech. Res. Commun. 130, 104114 (2023).
8. S. Arabnejad, D. Pasini, Mechanical properties of lattice materials via asymptotic homogenization and comparison with alternative homogenization methods. Int. J. Mech. Sci. 77, 249-262 (2013).
9. K. Liu, R. Sun, C. Daraio, Growth rules for irregular architected materials with programmable properties. Science 377, 975-981 (2022).
10. M. Zaiser, S. Zapperi, Disordered mechanical metamaterials. Nat. Rev. Phys. 5, 679-688 (2023).
11. M. Kadic, G. W. Milton, M. van Hecke, M. Wegener, 3D metamaterials. Nat. Rev. Phys. 1, 198-210 (2019).
12. R. Lakes, Foam Structures with a Negative Poisson's Ratio. Science 235, 1038-1040 (1987).
13. S. Babaee, J. Shim, J. C. Weaver, E. R. Chen, N. Patel, K. Bertoldi, 3D soft metamaterials with negative Poisson's ratio. Adv. Mater 25, 5044-5049 (2013).
14. T. Frenzel, M. Kadic, M. Wegener, Three-dimensional mechanical metamaterials with a twist. Science 358, 1072-1074 (2017).
15. J. Boddapati, M. Flaschel, S. Kumar, L. De Lorenzis, C. Daraio, Single-test evaluation of directional elastic properties of anisotropic structured materials. J. Mech. Phys. Solids 181, 105471 (2023).
16. B. Florijn, C. Coulais, M. van Hecke, Programmable Mechanical Metamaterials. Phys. Rev. Lett. 113, 175503 (2014).
17. K. Bertoldi, V. Vitelli, J. Christensen, M. van Hecke, Flexible mechanical metamaterials. Nat. Rev. Mater. 2, 17066 (2017).
18. C. Coulais, A. Sabbadini, F. Vink, M. van Hecke, Multi-step self-guided pathways for shape-changing metamaterials. Nature 561, 512-515 (2018).
19. X. Xia, A. Afshar, H. Yang, C. M. Portela, D. M. Kochmann, C. V. Di Leo, J. R. Greer, Electrochemically reconfigurable architected materials. Nature 573, 205-213 (2019).
20. X. Xia, C. M. Spadaccini, J. R. Greer, Responsive materials architected in space and time. Nat. Rev. Mater. 7, 683-701 (2022).
21. S. Shan, S. H. Kang, J. R. Raney, P. Wang, L. Fang, F. Candido, J. A. Lewis, K. Bertoldi, Multistable architected materials for trapping elastic strain energy. Adv. Mater 27, 4296-4301 (2015).
22. B. Haghpanah, L. Salari-Sharif, P. Pourrajab, J. Hopkins, L. Valdevit, Multistable shape-reconfigurable architected materials. Adv. Mater 28, 7915-7920 (2016).
23. S. Li, H. Fang, S. Sadeghi, P. Bhovad, K. Wang, Architected origami materials: how folding creates sophisticated mechanical properties. Adv. Mater. 31, 1805282 (2019).
24 J. Liu, T. Gu, S. Shan, S. H. Kang, J. C. Weaver, K. Bertoldi, Harnessing buckling to design architected materials that exhibit effective negative swelling. Adv. Mater 28, 6619-6624 (2016).
25. M. Kadic, T. Bückmann, N. Stenger, M. Thiel, M. Wegener, On the practicability of pentamode mechanical metamaterials. Appl. Phys. Lett. 100 (2012).
26. T. Bückmann, M. Thiel, M. Kadic, R. Schittny, M. Wegener, An elasto-mechanical unfeelability cloak made of pentamode metamaterials. Nat. Commun. 5, 4130 (2014).
27. Y. Estrin, A. V Dyskin, E. Pasternak, Topological interlocking as a material design concept. Mater. Sci. Eng. C 31, 1189-1194 (2011).
28. C. Chong, M. A. Porter, P. G. Kevrekidis, C. Daraio, Nonlinear coherent structures in granular crystals. J. Phys. Condens. Matter 29, 413003 (2017).
29 A. N. Karuriya, F. Barthelat, Granular crystals as strong and fully dense architectured materials. Proc. Natl. Acad. Sci. 120, e2215508120 (2023).
30. Y. Wang, L. Li, D. Hofmann, J. E. Andrade, C. Daraio, Structured fabrics with tunable mechanical properties. Nature 596, 238-243 (2021).
31. P. Tang, S. Coros, B. Thomaszewski, Beyond Chainmail: Computational Modeling of Discrete Interlocking Materials. ACM Trans. Graph. 42, 1-12 (2023).
32. Y. Liu, M. O'Keeffe, M. M. J. Treacy, O. M. Yaghi, The geometry of periodic knots, polycatenanes and weaving from a chemical perspective: A library for reticular chemistry. Chem. Soc. Rev. 47, 4642-4664 (2018).
33. L. Carlucci, G. Ciani, D. M. Proserpio, Polycatenation, polythreading and polyknotting in coordination network chemistry. Coord. Chem. Rev. 246, 247-289 (2003).
34 M. O'Keeffe, M. M. J. Treacy, Embeddings of Graphs: Tessellate and Decussate Structures. Int. J. Topol. 1, 1-10 (2024).
35. T. S. Lumpe, T. Stankovic, Exploring the property space of periodic cellular structures based on crystal networks. Proc. Natl. Acad. Sci. 118, e2003504118 (2021).
36. RCSR. https://rcsr.anu.edu.au/.
37. Materials and Methods are available as Supplementary Materials on Science Online.
38. E. Brown, A. Nasto, A. G. Athanassiadis, H. M. Jaeger, Strain stiffening in random packings of entangled granular chains. Phys. Rev. Lett. 108, I-4 (2012).
39 R. Kawamoto, E. Andò, G. Viggiani, J. E. Andrade, Level set discrete element method for three-dimensional computations with triaxial case study. J. Mech. Phys. Solids 91, 1-13 (2016).
40. R. Kawamoto, E. Andò, G. Viggiani, J. E. Andrade, All you need is shape: Predicting shear banding in sand with LS-DEM. J. Mech. Phys. Solids 111, 375-392 (2018).
41. L. Li, E. Marteau, J. E. Andrade, Capturing the inter-particle force distribution in granular material using LS-DEM. Granul. Matter 21, 1-16 (2019).
42. A. J. Liu, S. R. Nagel, The jamming transition and the marginally jammed solid. Annu. Rev. Condens. Matter Phys. 1, 347-369 (2010).
43. T. C. de Goede, K. G. de Bruin, D. Bonn, High-velocity impact of solid objects on Non-Newtonian Fluids. Sci. Rep. 9, 1250 (2019).
44. H. M. Laun, Rheological properties of aqueous polymer dispersions. Die Angew. Makromol. Chemie Appl. Macromol. Chem. Phys. 123, 335-359 (1984).
45. C. K. K. Lun, S. B. Savage, D. J. Jeffrey, N. Chepurniy, Kinetic theories for granular flow: inelastic particles in Couette flow and slightly inelastic particles in a general flowfield. J. Fluid Mech. 140, 223-256 (1984).
46. F. Da Cruz, S. Emam, M. Prochnow, J.-N. Roux, F. Chevoir, Rheophysics of dense granular materials: Discrete simulation of plane shear flows. Phys. Rev. E 72, 21309 (2005).
47. L. Li, J. E. Andrade, Identifying spatial transitions in heterogenous granular flow. Granul. Matter 22, 52 (2020).
48. W. Losert, L. Bocquet, T. C. Lubensky, J. P. Gollub, Particle dynamics in sheared granular matter. Phys. Rev. Lett. 85, 1428 (2000).

1. A structure, comprising:
a plurality of unit particles, each unit particle comprising a particle body formed from one or more structural members and defining one or more interlocking openings at one or more nodes;
wherein the plurality of unit particles are interconnected to form a three-dimensional network patterned after a crystalline network topology and comprising inter-layer linkages;
wherein an interconnection between adjacent unit particles is a mechanical interlock formed by an interlocking structural member of one unit particle passing through an interlocking opening of an adjacent unit particle at a node; and
wherein adjacent particle bodies are not bonded and are topologically inseparable absent cutting or fracture.

2. The structure of claim 1, wherein the interconnection provides three-dimensional cohesion to the structure, the three-dimensional cohesion arising from mechanical interlocks at nodes and comprising inter-layer linkages.

3. The structure of claim 1, wherein the structure is configured to exhibit a dual mechanical response, transitioning from a fluid-like regime at strains below a critical jamming strain to a solid-like regime at strains above the critical jamming strain.

4. The structure of claim 3, wherein the fluid-like regime is characterized by a near-zero shear modulus and shear-thinning behavior.

5. The structure of claim 3, wherein the solid-like regime is characterized by a nonlinear stress-strain response and shear-thickening or strain-stiffening behavior.

6. The structure of claim 3, wherein the critical jamming strain is a tunable parameter determined by a selection of one or more of the crystalline network topology, a particle body geometry, or a structural member dimension.

7. The structure of claim 6, wherein the crystalline network topology is a J-type topology, the particle body geometry is a toroidal ring, and the structure is configured to exhibit a critical shear jamming strain (γs*) between approximately 30% and 70%.

8. The structure of claim 6, wherein the crystalline network topology is a T-type topology, the particle body geometry is a toroidal ring, and the structure is configured to exhibit a critical compressive jamming strain (ϵc*) between approximately 10% and 28%.

9. The structure of claim 6, wherein the particle body geometry is polyhedral, and the critical jamming strain is lower than for a particle body geometry that is toroidal within the same crystalline network topology.

10. The structure of claim 6, wherein the structural member dimension is a thickness-to-diameter (d/D) ratio, and wherein the critical jamming strain is inversely related to the d/D ratio.

11. The structure of claim 1, wherein each unit particle has a maximum side length of 1 mm.

12. The structure of claim 11, wherein each unit particle comprises a conductive material or is coated with a conductive material.

13. The structure of claim 12, wherein the conductive material is a coating of copper at a thickness of approximately 300 nm.

14. The structure of claim 1, wherein the plurality of unit particles are formed from a constituent material selected to be responsive to a trigger selected from the group consisting of: a magnetic field, a thermal change, a solvent, humidity, and light.

15. The structure of claim 14, wherein the constituent material is a ferromagnetic-doped polymer, and the structure is configured to convert from a first state to a second state upon application of a magnetic field.

16. The structure of claim 14, wherein the constituent material is a shape memory polymer, and the structure is configured to convert from a first state to a second state upon application of a thermal change.

17. The structure of claim 14, wherein the constituent material is a solvent-responsive polymer or a hydrogel, and the structure is configured to convert from a flexible state to a rigid state upon exposure to a solvent or humidity.

18. The structure of claim 1, wherein the crystalline network topology is a J-4-square topology, and the structure is configured to be multi-stable, exhibiting at least a first expanded stable configuration and a second collapsed stable configuration.

19-72. (canceled)

73. The structure of claim 1, wherein the structure exhibits orientation-dependent anisotropy.

74. The structure of claim 73, wherein the orientation-dependent anisotropy is characterized by the structure settling into different relaxed outlines under gravity based on its crystallographic orientation.

75-81. (canceled)

82. An impact protection system, comprising: a component configured to absorb energy; wherein the component comprises a structure according to claim 1.

83. The impact protection system of claim 82, wherein the system is selected from the group consisting of: a protective case, a shock absorber, a vehicle crash structure, a blast protection system, a seismic damper, a helmet, and body armor.

84. A soft robotic system, comprising: at least one reconfigurable component; wherein the at least one reconfigurable component comprises a structure according to claim 1.

85. A morphing architecture, comprising: at least one reconfigurable component; wherein the at least one reconfigurable component comprises a structure according to claim 1.

86. A flexible medical implant, comprising: a structure according to claim 1.
